(19) United States
(12) Patent Application Publication
(10) Pub. No.: US 2026/0008764 A1
(43) Pub. Date: Jan. 8, 2026

(54) BACKBONE CLEAVABLE POLYMETHACRYLATES VIA THIONOLACTONE COMONOEMERS

(71) Applicant: Massachusetts Institute of Technology, Cambridge, MA (US)

(72) Inventors: Jeremiah A. Johnson, Boston, MA (US); Kwangwook Ko, Cambridge, MA (US)

(73) Assignee: Massachusetts Institute of Technology, Cambridge, MA (US)

(21) Appl. No.: 19/126,358

(22) PCT Filed: Aug. 13, 2024
(86) PCT No.: PCT/US2024/042172

§ 371 Date: May 1, 2025

Related U.S. Application Data
(60) Provisional application No. 63/519,455, filed on Aug. 14, 2023.

(51) Int. Cl. C07D 313/14

(52) U.S. Cl. CPC C07D 313/14

(57) ABSTRACT

The present disclosure provides thionolactones (e.g., compounds of Formula II, and tautomers and salts thereof). The thionolactones may be useful as comonomers to copolymerize with other comonomers, e.g., methacrylates (e.g., MMA) to generate copolymers, e.g., random copolymers. The copolymers may be degradable (e.g., backbone degradable). The copolymers may be useful for waste management or biodegradability.

### RELATED APPLICATIONS

The present patent application claims priority to U.S. Provisional Patent Application No. 63/519,455, filed Aug. 14, 2023, which is incorporated herein by reference in its entirety.

### BACKGROUND OF THE INVENTION

There has been a need for the introduction of backbone deconstructability into polymers in the development of cleavable comonomers (CCs) with suitable copolymerization reactivity. Recent advancements in thionolactone-based CCs, exemplified by dibenzo[c,e]-oxepine-5(7H)-thione (DOT), have opened avenues for the efficient and selective deconstruction of multiple classes of vinyl polymers, including polyacrylates, polyacrylamides, and polystyrenics. To date, however, no thionolactone CC has been shown to copolymerize with methacrylates to an appreciable extent to enable appreciable polymer deconstruction.

### SUMMARY OF THE PRESENT DISCLOSURE

Thionolactones are monomers for radical ring-opening polymerization (rROP), allowing one to install cleavable thioester groups into the backbones of polymers and thereby make them degradable into small fragments for recycling, upcycling, and biodegradation schemes.

However, to date, there are no literature reports of thionolactones or other cleavable comonomers that have been successful for poly(methacrylates), which are an important class of polymers (e.g., polymethylmethacrylate (PMMA) and polymethacrylic acid (PMAA)). This challenge may be because of the reaction kinetics of methacrylate polymerization: the very fast propagation of methacrylate radicals with methacrylate monomers (“homopropagation”) may make it difficult for comonomers to compete with homopropagation, and thus, previously cleavable comonomers may not insert into the polymer backbone statistically or randomly, leading instead to block structures. If more cleavable bonds are in separate blocks, then it is less likely to break down the methacrylate backbone substantially.

In one aspect, the present disclosure provides thionolactones (e.g., compounds of Formula II, and tautomers and salts thereof). The thionolactones may be useful as comonomers to copolymerize with other comonomers, e.g., methacrylates (e.g., MMA) to generate copolymers, e.g., random copolymers. The copolymers may be degradable (e.g., backbone degradable). The copolymers may be useful for waste management or biodegradability.

In another aspect, the present disclosure provides a compound of the Formula II:

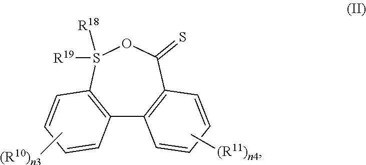

or a tautomer or salt thereof, wherein:
each instance of R¹⁰ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R¹¹ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R^{b} is independently hydrogen, substituted or unsubstituted alkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;
each of n3 and n4 is independently 0, 1, 2, 3, or 4;
R¹⁸ is substituted or unsubstituted aryl, substituted or unsubstituted alkyl, substituted or unsubstituted carbocyclyl, substituted or unsubstituted heterocyclyl, or substituted or unsubstituted heteroaryl; and
R¹⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl; provided that no instance of R¹⁰, R¹¹, R¹⁸, and R¹⁹ comprises one or more non-aromatic unsaturated CC bonds.

In another aspect, the present disclosure provides a copolymer comprising:
m1 instances of a first type of repeating units of Formula i′:

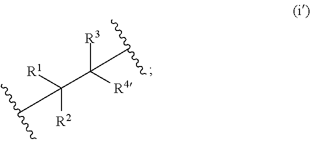

m2 instances of a second type of repeating units of Formula ii:

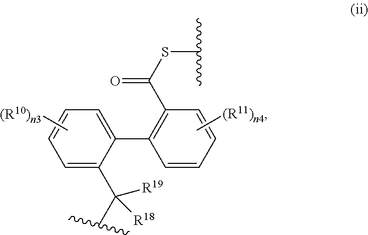

or a tautomer thereof;
optionally one or more types of crosslinking units; and
optionally one or more types of additional repeating units;
wherein:
m1 is an integer between 10 and 1,000,000, inclusive;
m2 is an integer between 2 and 1,000,000, inclusive;
R¹, R², and R³ are each independently hydrogen, halogen, or substituted or unsubstituted alkyl;
R⁴′ is —C(═O)OR^{a}, —C(═O)N(R^{a})₂, substituted or unsubstituted alkyl, halogen, substituted or unsubstituted carbocyclyl, substituted or unsubstituted heterocyclyl, substituted or unsubstituted aryl, substituted or unsubstituted heteroaryl, —CN, —OR^{a}, —SCN, —SR^{a}, —SSR^{a}, —N₃, —NO, —N(R^{a})₂, —NO₂, —C(═O)R^{a}, —C(═O)SR^{a}, —C(═NR^{a})R^{a}, —C(═NR^{a})OR^{a}, —C(═NR^{a})SR^{a}, —C(═NR^{a})N(R^{a})₂, —S(═O)R^{a}, —S(═O)OR^{a}, —S(═O)SR^{a}, —S(═O)N(R^{a})₂, —S(═O)₂R^{a}, —S(═O)₂OR^{a}, —S(═O)₂SR^{a}, —S(═O)₂N(R^{a})₂, —OC(═O)R^{a}, —OC(═O)OR^{a}, —OC(═O)SR^{a}, —OC(═O)N(R^{a})₂, —OC(═NR^{a})R^{a}, —OC(═NR^{a})OR^{a}, —OC(═NR^{a})SR^{a}, —OC(═NR^{a})N(R^{a})₂, —OS(═O)R^{a}, —OS(═O)OR^{a}, —OS(═O)SR^{a}, —OS(═O)N(R^{a})₂, —OS(═O)₂R^{a}, —OS(═O)₂OR^{a}, —OS(═O)₂SR^{a}, —OS(═O)₂N(R^{a})₂, —ON(R^{a})₂, —SC(═O)R^{a}, —SC(═O)OR^{a}, —SC(═O)SR^{a}, —SC(═O)N(R^{a})₂, —SC(═NR^{a})R^{a}, —SC(═NR^{a})OR^{a}, —SC(═NR^{a})SR^{a}, —SC(═NR^{a})N(R^{a})₂, —NR^{a}C(═O)R^{a}, —NR^{a}C(═O)OR^{a}, —NR^{a}C(═O)SR^{a}, —NR^{a}C(═O)N(R^{a})₂, —NR^{a}C(═NR^{a})R^{a}, —NR^{a}C(═NR^{a})OR^{a}, —NR^{a}C(═NR^{a})SR^{a}, —NR^{a}C(═NR^{a})N(R^{a})₂, —NR^{a}S(═O)R^{a}, —NR^{a}S(═O)OR^{a}, —NR^{a}S(═O)SR^{a}, —NR^{a}S(═O)N(R^{a})₂, —NR^{a}S(═O)₂R^{a}, —NR^{a}S(═O)₂OR^{a}, —NR^{a}S(═O)₂SR^{a}, —NR^{a}S(═O)₂N(R^{a})₂, —Si(R^{a})₃, —Si(R^{a})₂OR^{a}, —Si(R^{a})(OR^{a})₂, —Si(OR^{a})₃, —OSi(R^{a})₃, —OSi(R^{a})₂OR^{a}, —OSi(R^{a})(OR^{a})₂, or —OSi(OR^{a})₃;
each instance of R^{a} is independently hydrogen, substituted or unsubstituted alkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;
each instance of R¹⁰ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R¹¹ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R^{b} is independently hydrogen, substituted or unsubstituted alkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;
each of n3 and n4 is independently 0, 1, 2, 3, or 4;
R¹⁸ is substituted or unsubstituted aryl, substituted or unsubstituted alkyl, substituted or unsubstituted carbocyclyl, substituted or unsubstituted heterocyclyl, or substituted or unsubstituted heteroaryl; and
R¹⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
provided that no instance of R¹, R², R³, R⁴′, R¹⁰, R, R¹⁸, and R¹⁹ comprises one or more non-aromatic unsaturated CC bonds.

In another aspect, the present disclosure provides a composition comprising:
the compound, or a tautomer or salt thereof; and
optionally an excipient.

In another aspect, the present disclosure provides a composition comprising:
the copolymer; and
optionally an excipient.

In another aspect, the present disclosure provides a kit comprising:
the compound, or a tautomer or salt thereof, or the composition; and
instructions for using the compound, tautomer, salt, or composition.

In another aspect, the present disclosure provides a kit comprising:
the copolymer or the composition; and
instructions for using the copolymer or composition.

In another aspect, the present disclosure provides a method of preparing the copolymer comprising polymerizing the first type of monomers, the second type of monomers, optionally the crosslinkers, and optionally the additional types of monomers.

In another aspect, the present disclosure provides a method of degrading the copolymer comprising reacting the copolymer with a nucleophile.

The copolymers of the present disclosure may be advantageous over the known copolymers because the former show less pronounced compositional gradients, fewer unwanted crosslinks, increased compatibility with free-radical polymerization (FRP) methods, more efficient deconstruction without compromising material properties, more efficient degradation at low CC loadings, and/or no or less decreasing of the properties (e.g., mechanical properties) of the homopolymers prepared from the comonomers.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

In a formula, the bond [symbol] a is a single bond, the dashed line [symbol] is a single bond or absent, and the bond [symbol] or [symbol] is a single or double bond.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” is intended herein to include alkyl, alkenyl, alkynyl, cycloalkyl, cycloalkenyl, and cycloalkynyl moieties.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ═CH). The moieties ═CH₂ and ═CH are also alkyl.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl” or “C₁₋₂₀ heteroalkyl”). In certain embodiments, a heteroalkyl group refers to a saturated group having from 1 to 12 carbon atoms and 1 or more heteroatoms within the parent chain (“heteroC₁₋₁₂ alkyl”). In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂C₁, and the like.

“Alkenyl” refers to a radical of a straight-chain or branched hydrocarbon group having from 2 to 20 carbon atoms, one or more (e.g., two, three, or four, as valency permits) carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

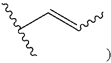

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl” or “C₂₋₂₀ heteroalkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and lor 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl” or “C₂₋₂₀ heteralkynyl”). In certain embodiments, a heteroalkynyl group refers to a group having from 2 to 12 carbon atoms, at least one triple bond, and 1 or more heteroatoms within the parent chain (“heteroC₂₋₁₂ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and lor 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C≡C triple bonds in the carbocyclic ring is referred to as “cycloalkynyl.” Carbocyclyl includes aryl. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-13 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Partially unsaturated heterocyclyl groups includes heteroaryl. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include azirdinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In some embodiments, the heteroalkyl, heteroalkenyl, and heteroalkynyl are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(=O)(R^{aa})₂, —NR^{bb}P(=O)(OR^{cc})₂, —NR^{bb}P(=O)(N(R^{bb})₂)₂, —P(R^{aa})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(=O)₂R^{aa}, ═NR^{bb}, or =NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{aa})₂—CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{cc})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{dd} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{cc}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{cc}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁹⁹ groups, or two geminal R^{dd} substituents can be joined to form O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻. HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₃⁻, B₄O₇²⁻, S₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{bb})₂—CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{aa})₂, —C(═O)R^{aa}, —C(═O)N(R^{aa})₂, —CO₂R^{aa}, —SO₂R^{aa}, —C(═NR^{cc})R^{aa}, —C(═NR^{cc})OR^{aa}, —C(═NR^{cc})N(R^{aa})₂, —SO₂N(R^{aa})₂, —SO₂R^{aa}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{aa})₃⁺X⁻, —P(OR^{cc})₂, —P(OR^{aa})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{aa})₂, —P(OR^{aa})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by subtracting the atomic weight of a hydrogen atom from the molecular weight of the molecule R—H. The “molecular weight” of -L-, wherein -L- is any divalent moiety, is calculated by subtracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

The term “leaving group” is given its ordinary meaning in the art of synthetic organic chemistry and refers to an atom or a group capable of being displaced by a nucleophile. Examples of suitable leaving groups include halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other examples of leaving groups are water, ammonia, alcohols, ether moieties, thioether moieties, zinc halides, magnesium moieties, diazonium salts, and copper moieties.

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods known in the art such as ion exchange.

Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

“Compounds” include, e.g., small molecules and macromolecules. Macromolecules include, e.g., polymers, peptides, proteins, carbohydrates, monosaccharides, oligosaccharides, polysaccharides, nucleoproteins, mucoproteins, lipoproteins, synthetic polypeptides or proteins, glycoproteins, steroids, nucleic acids, DNAs, RNAs, nucleotides, nucleosides, oligonucleotides, antisense oligonucleotides, lipids, hormones, vitamins, and cells.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 g/mol, not more than 900 g/mol, not more than 800 g/mol, not more than 700 g/mol, not more than 600 g/mol, not more than 500 g/mol, not more than 400 g/mol, not more than 300 g/mol, not more than 200 g/mol, or not more than 100 g/mol. In certain embodiments, the molecular weight of a small molecule is at least 100 g/mol, at least 200 g/mol, at least 300 g/mol, at least 400 g/mol, at least 500 g/mol, at least 600 g/mol, at least 700 g/mol, at least 800 g/mol, or at least 900 g/mol, or at least 1,000 g/mol. Combinations of the above ranges (e.g., at least 200 g/mol and not more than 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “polymer” refers to a compound comprising eleven or more covalently connected repeating units. In certain embodiments, a polymer is naturally occurring. In certain embodiments, a polymer is synthetic (e.g., not naturally occurring). In certain embodiments, the M_{W} of a polymer is between 1,000 and 2,000, between 2,000 and 10,000, between 10,000 and 30,000, between 30,000 and 100,000, between 100,000 and 300,000, between 300,000 and 1,000,000, g/mol, inclusive. In certain embodiments, the M_{W} of a polymer is between 2,000 and 1,000,000, g/mol, inclusive. A polymer may be a copolymer.

The term “average molecular weight” may encompass the number average molecular weight (Mₙ), weight average molecular weight (M_{w}), higher average molecular weight (M_{z} or M_{z}+1), GPC/SEC (gel permeation chromatography/size-exclusion chromatography)-determined average molecular weight (Mₚ), and viscosity average molecular weight (Mᵥ). Average molecular weight may also refer to average molecular weight as determined by gel permeation. Average molecular weight may also refer to average molecular weight as determined by size exclusion chromatography.

The term “degree of polymerization” (DP) refers to the number of repeating units in a polymer. In certain embodiments, the DP is determined by a chromatographic method, such as gel permeation chromatography. For a homopolymer, the DP refers to the number of repeating units included in the homopolymer. For a copolymer of two types of monomers (e.g., a first monomer and a second monomer) wherein the molar ratio of the two types of monomers is about 1:1, the DP refers to the number of repeating units of either one of the two type of monomers included in the copolymer. For a copolymer of two types of monomers (e.g., a first monomer and a second monomer) wherein the molar ratio of the two types of monomers is not about 1:1, two DPs may be used. A first DP refers to the number of repeating units of the first monomer included in the copolymer, and a second DP refers to the number of repeating units of the second monomer included in the copolymer. Unless provided otherwise, a DP of “xx”, wherein xx is an integer, refers to the number of repeating units of either one of the two types of monomers of a copolymer of two types of monomers (e.g., a first monomer and a second monomer) wherein the molar ratio of the two types of monomers is about 1:1. Unless provided otherwise, a DP of “xx-yy”, wherein xx and yy are integers, refers to xx being the number of repeating units of the first monomer, and yy being the number of repeating units of the second monomer, of a copolymer of two types of monomers (e.g., a first monomer and a second monomer) wherein the molar ratio of the two types of monomers is not about 1:1.

The term “v/v” refers to volume per volume and is used herein to express concentrations of monomers. Unless otherwise provided, a percent concentration of a second monomer in a first monomer is expressed in v/v. For example, a mixture of a first monomer and 10% second monomer refers to a mixture of a first monomer and a second monomer, wherein the volume of the second monomer is 10% of the combined volumes of the first and second monomers.

The disclosure is not intended to be limited in any manner by the above exemplary listing of substituents. Additional terms may be defined in other sections of this disclosure.

### BRIEF DESCRIPTION OF THE DRAWINGS

The figures are exemplary and do not limit the scope of the present disclosure.

FIG. 1 shows DFT calculations for MMA radical addition to thionolactone (DOT) derivative showing that TS2 is rate-limiting step.

FIG. 2 shows stabilizing TS2 with benzylic functional groups should lower the barrier to copolymerization. The effect of benzylic substituent(s) on the energy profile was evaluated using DFT calculations. The calculations employed the wB97X-D3/def2-DZVP level of theory, and the electronic energies of all optimized structures were reevaluated using wB97X-D3/def2-TZVP.

FIG. 3 shows synthesis of F-p-CF₃PhDOT: a thionolactone with benzylic substitution.

FIG. 4 shows synthesis of PFPhDOT: another thionolactone with benzylic substitution.

FIG. 5 shows kinetic experiments for various benzylic “PhDOT” derivatives showing consumption of both DOT and MMA. Derivatives depicted are 10% PhDOT, 10% p-MePhDOT, 10% p-FPhDOT, 10% Me-p-CF₃PhDOT, 10% p-CF₃PhDOT.

FIG. 6 shows kinetic experiments for various benzylic “PhDOT” derivatives showing consumption of both DOT and MMA. Derivatives depicted are 10% F-p-CF₃PhDOT and 10% PFPhDOT.

FIG. 7 shows that Meyer-Lowry fitting of the low-conversion kinetic data gives reactivity ratios.

FIG. 8 shows that F-p-CF₃PhDOT copolymerizes with MMA under bulk free radical conditions (SEC data).

FIG. 9 shows that copolymers of F-p-CF₃PhDOT with MMA are degradable as shown by SEC.

FIG. 10 shows that fragment size of F-p-CF₃PhDOT with MMA copolymer degradation depends on DOT loading.

FIG. 11 shows that F-p-CF₃PhDOT with MMA copolymers degrades in the presence of propylamine; PMMA does not.

FIG. 12 shows similar findings for PFPhDOT with MMA.

FIG. 13 shows further similar findings for PFPhDOT with MMA.

FIG. 14 shows similar findings for PFPhDOT with MMA.

FIG. 15 shows similar findings for PFPhDOT with MMA.

FIG. 16 shows that PMMA crosslinked networks with thionolactone are dissolvable with propylamine.

FIGS. 17A and 17B. FIG. 17A shows Monte Carlo simulation evaluating the dependence of the efficiency of CCs on reactivity ratios. The simulations were done assuming 2.5% CC loading and a degree of polymerization (DP) of 1,000. The colors reflect the size of the degraded fragments relative to the size of the original polymer. Regions I-V represent different reactivity ratio scenarios. FIG. 17B shows examples of simulated polymer sequences for Regions I-V: the horizontal black lines represent the polymers, grown from left to right; the vertical lines indicate the positions of CCs on the polymer chain.

FIG. 18 shows that a relative Gibbs free energy profile for an MMA radical reacting with either MMA or DOT was calculated in order to model the homopropagation and crosspropagation of a chain ending with MMA.

FIGS. 19A to 19D show synthetic routes which have been developed to synthesize bDOTs with either (FIG. 19A) one or (FIG. 19B) two functional groups at the benzylic position (indicated by a circle marked with an asterisk). Simultaneous functionalization of the aryl ring (circles with no asterisk) can be achieved using appropriate starting materials. FIG. 19C shows overall yields of bDOT synthesis. FIG. 19D shows the copolymerization reactivity of the bDOTs with MMA was measured under RAFT conditions. F_{bDOT} values were determined by comparing the integrations of the aromatic C—H peaks with the MMA O—CH₃ peaks in ¹H NMR spectra. Molar masses were determined by analytical SEC and referenced against PMMA standards.

FIGS. 20A to 20C. FIG. 20A shows a series of bDOTs which were synthesized for the optimization of copolymerization reactivity. FIG. 20B shows that the conversion of the monomers was measured over time using quantitative ¹H NMR. FIG. 20C shows reactivity ratios which were determined by fitting the conversion data to the Meyer-Lowry equation.

FIG. 21 shows that Monte Carlo simulation evaluates the effectiveness of aryl bDOTs as CCs. The heat map generated from the simulation visualizes the ratio of degraded fragment size to copolymer size as a function of reactivity ratios, at a 2.5 mol % CC loading for DP 1000 copolymers.

FIGS. 22A to 22C. FIG. 22A shows that bulk free-radical copolymerization of MMA and F-p-CF₃PhDOT yields copolymers with variable composition, termed dPMMA(f_{bDOT}). These copolymers were evaluated for their degradation into OMMA(f_{bDOT}) under an aminolysis condition. FIG. 22B shows NMR spectra of the copolymers. F_{bDOT} values were determined by comparing the integrations of the aromatic C—H peaks (indicated by dashed rectangle) and MMA O—CH₃ peaks (indicated by dotted circle). The integrations are shown below each spectrum. FIG. 22C shows SEC traces and M_{n,SEC} for vPMMA, dPMMA(f_{bDOT}), and OMMA(f_{bDOT}). Molar masses from SEC were referenced to PMMA standards.

FIGS. 23A to 23C. FIG. 23A shows DSC traces from the second heating ramp at 10° C./minute. FIG. 23B shows TGA traces acquired at 10° C./minute. FIG. 23C shows DMA temperature sweeps at constant amplitude and frequency.

FIGS. 24A to 24B show Mulliken spin population analysis of TS-D2 for DOT. FIG. 24A shows CYLview visualization displaying spin populations of heavy atoms. Spin populations on hydrogen atoms are omitted for clarity. FIG. 24B shows a cartoon representation; the areas of the circles are proportional to the spin population on each atom. The circles outlined with a dotted line are positive; the circles with no outline are negative.

FIG. 25 shows a summary of the synthesis of MeDOT and PhDOT.

FIG. 26 shows a summary of the synthesis of Me₂DOT.

FIG. 27 shows a summary of the synthesis of p-MePhDOT.

FIG. 28 shows a summary of the synthesis of p-CF₃PhDOT.

FIG. 29 shows a summary of the synthesis of F-p-CF₃PhDOT.

FIG. 30 shows a summary of the synthesis of PFPhDOT.

FIGS. 31A to 31B show conversion measured versus reaction time for the copolymerization of (FIG. 31A) MeDOT or (FIG. 31B) Me₂DOT with MMA.

FIG. 32 shows conversion measured over reaction time for the copolymerization of PhDOT and MMA.

FIG. 33 shows ¹H NMR (400 MHz, CDCl₃) of P(MMA-co-PhDOT).

FIG. 34 shows ¹³C{¹H} NMR (101 MHz, CDCl₃) of P(MMA-co-PhDOT).

FIG. 35 shows a contour plot of a ¹H-¹³C HSQC NMR spectrum of P(MMA-co-PhDOT).

FIG. 36 shows SEC analysis of the purified polymers (A) and the fragments post-degradation (B). The molecular weights and dispersities of the polymers are summarized in Table 2. The traces have been normalized based on their respective areas.

FIG. 37 shows a control deconstruction experiment using vPMMA.

FIG. 38 shows screening for deconstruction conditions using dPMMA(2.5): All aminolysis conditions resulted in deconstructed fragments with the same molecular weight distribution.

FIGS. 39A to 39B show ¹H NMR analysis for the evaluation of deconstructed fragments.

FIG. 40 shows monomer conversions measured at different timepoints for bulk free-radical copolymerization of F-p-CF₃PhDOT and MMA.

FIG. 41 shows a comparison of the performance of bDOTs (PhDOT and F-p-CF₃PhDOT) as CCs in bulk free-radical copolymerizations.

FIG. 42 shows synthesis and deconstruction of dPMMA using AIBN as an initiator.

FIG. 43 shows a graphical overview depicting copolymerization and deconstruction.

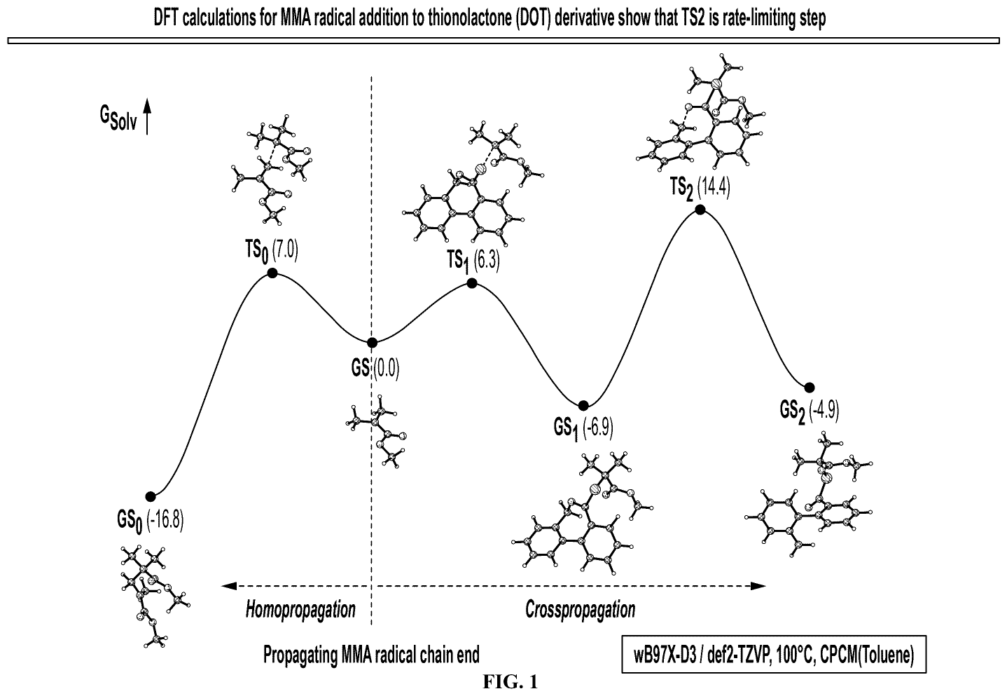

### DETAILED DESCRIPTION OF CERTAIN EMBODIMENTS OF THE PRESENT DISCLOSURE

In one aspect, the present disclosure provides a compound of the Formula II:

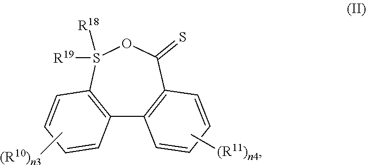

or a tautomer or salt thereof, wherein:
each instance of R¹⁰ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R¹¹ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R^{b} is independently hydrogen, substituted or unsubstituted alkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;
each of n3 and n4 is independently 0, 1, 2, 3, or 4;
R¹⁸ is substituted or unsubstituted aryl, substituted or unsubstituted alkyl, substituted or unsubstituted carbocyclyl, substituted or unsubstituted heterocyclyl, or substituted or unsubstituted heteroaryl; and
R¹⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
provided that no instance of R¹⁰, R¹¹, R¹⁸, and R¹⁹ comprises one or more non-aromatic unsaturated CC bonds.

In certain embodiments, the compound is of the formula:

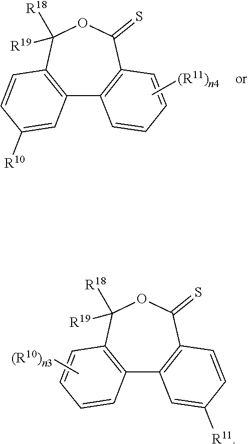

In certain embodiments, the compound is of the formula:

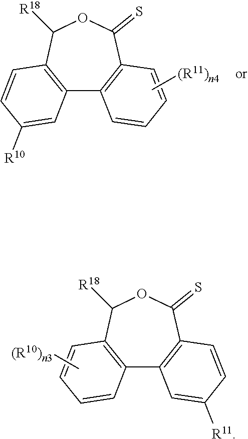

In certain embodiments, the compound is of the formula:

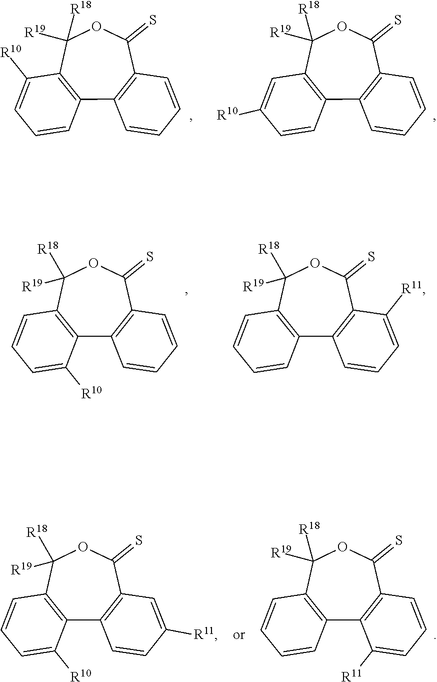

In certain embodiments, the compound is of the formula:

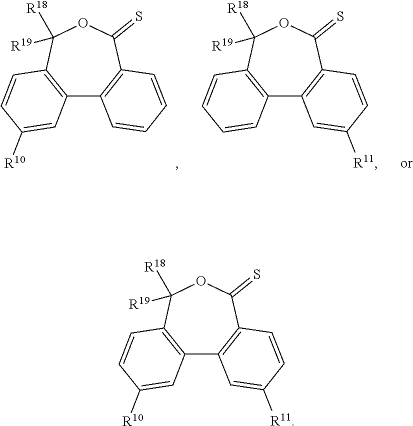

In certain embodiments, the compound is of the formula:

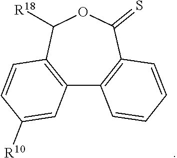

In certain embodiments, the compound is of the formula:

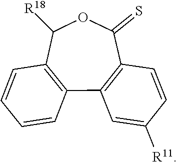

In certain embodiments, the compound is of the formula:

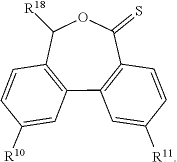

In certain embodiments, n3 is 1. In certain embodiments, n3 is 0.

In certain embodiments, n4 is 0. In certain embodiments, n4 is 1.

In certain embodiments, at least one of n3 and n4 is 1, 2, 3, or 4. In certain embodiments, at least one of n3 and n4 is 1. In certain embodiments, each of n3 and n4 is 1, 2, 3, or 4. In certain embodiments, each of n3 and n4 is 1.

In certain embodiments, “at least one” is “two.” In certain embodiments, “at least one instance” is “two instances.” In certain embodiments, “at least one” is “each.” In certain embodiments, “at least one instance” is “each instance.” In certain embodiments, two or more instances of the same moiety (e.g., R¹⁰, R¹¹, R^{a}, R^{b}, or R^{c}) are the same as each other. In certain embodiments, two or more instances of the same moiety are different from each other.

In certain embodiments, at least one instance of R¹⁰ or R¹¹ is an electron-withdrawing group. In certain embodiments, at least one instance of R¹⁰ or R¹¹ is more electron-withdrawing than —H. In certain embodiments, at least one instance of R¹⁰ or R¹¹ is not more electron-withdrawing than —CF₃.

In certain embodiments, at least one instance of R¹⁰ or R¹¹ is halogen, preferably, fluoro. In certain embodiments, at least one instance of R¹⁰ or R¹¹ is —Cl. In certain embodiments, at least one instance of R¹⁰ or R¹¹ is halogen, substituted or unsubstituted, C₁₋₆ alkyl, —OR^{aa}, —SR^{aa}, —N(R^{cc})₂, —CN, —SCN, —NO₂, —C(═O)R^{aa}, —C(═O)OR^{aa}, —C(═O)N(R^{c})₂, —OC(═O)R^{aa}, —OC(═O)OR^{c}, —OC(═O)N(R^{c})₂, —NR^{c}C(═O)R^{aa}, —NR^{c}C(═O)OR^{aa}, or —NR^{c}C(═O)N(R^{c})₂, wherein each instance of R^{c} is independently hydrogen or substituted or unsubstituted C₁₋₆ alkyl. In certain embodiments, at least one instance of R¹⁰ or R¹¹ is halogen, —CN, —SCN, —NO₂, —C(═O)R^{c}, —C(═O)OR^{aa}, —C(═O)N(R^{c})₂, —OC(═O)R^{aa}, —OC(═O)OR^{aa}, —OC(═O)N(R^{c}), —NR^{c}C(═O)R^{c}, —NR^{c}C(═O)OR^{c}, —NR^{c}C(═O)N(R^{c})₂, or C₁₋₆ alkyl substituted with one or more halogen, wherein each instance of R^{c} is independently hydrogen or substituted or unsubstituted C₁₋₆ alkyl. In certain embodiments, at least one instance of R¹⁰ or R¹¹ is substituted or unsubstituted alkyl, —O(substituted or unsubstituted alkyl), or —S(substituted or unsubstituted alkyl). In certain embodiments, at least one instance of R¹⁰ or R¹¹ is unsubstituted C₁₋₆ alkyl, C₁₋₆ alkyl substituted with one or more fluoro, —O(unsubstituted C₁₋₆ alkyl), —O(C₁₋₆ alkyl substituted with one or more fluoro), —S(unsubstituted C₁₋₆ alkyl), or —S(C₁₋₆ alkyl substituted with one or more fluoro). In certain embodiments, at least one instance of R¹⁰ or R¹¹ is —CH₃. In certain embodiments, at least one instance of R¹⁰ or R¹¹ is —CH₂F, —CHF₂, or —CF₃. In certain embodiments, at least one instance of R¹⁰ or R¹¹ is —CN.

In certain embodiments, R¹⁸ is substituted or unsubstituted aryl. In certain embodiments, R¹⁸ is substituted or unsubstituted phenyl. In certain embodiments, R¹⁸ is unsubstituted phenyl. In certain embodiments, R¹⁸ is para-monosubstituted phenyl. In certain embodiments, R¹⁸ is ortho-monosubstituted phenyl. In certain embodiments, R¹⁸ is meta-monosubstituted phenyl. In certain embodiments, R¹⁸ is polysubstituted phenyl. In certain embodiments, R¹⁸ is disubstituted phenyl. In certain embodiments, R¹⁸ is persubstituted phenyl. In certain embodiments, R¹⁸ is unsubstituted phenyl or phenyl substituted with one or more: halogen, substituted or unsubstituted, C₁₋₆ alkyl, —OR^{c}, —SR^{c}, —N(R^{c})₂, —CN, —SCN, —NO₂, —C(═O)R^{c}, —C(═O)OR^{c}, —C(═O)N(R^{c})₂, —OC(═O)R^{c}, —OC(═O)OR^{c}, —OC(═O)N(R^{c})₂, —NR^{c}C(═O)R^{c}, —NR^{c}C(═O)OR^{c}, and/or —NR^{c}C(═O)N(R^{c})₂, wherein each instance of R^{c} is independently hydrogen or substituted or unsubstituted C₁₋₆ alkyl. In certain embodiments, R¹⁸ is unsubstituted phenyl or phenyl substituted with one or more: halogen, —CN, —SCN, —NO₂, —C(═O)R^{c}, —C(═O)OR^{c}, —C(═O)N(R^{c})₂, —OC(═O)R^{c}, —OC(═O)OR^{c}, —OC(═O)N(R^{c})₂, —NR^{c}C(═O)R^{c}, —NR^{c}C(═O)OR^{c}, —NR^{c}C(═O)N(R^{c})₂, and/or C₁₋₆ alkyl substituted with one or more halogen, wherein each instance of R^{c} is independently hydrogen or substituted or unsubstituted C₁₋₆ alkyl. In certain embodiments, R¹⁸ is unsubstituted phenyl or phenyl substituted with one or more: halogen, unsubstituted C₁₋₆ alkyl, and/or C₁₋₆ alkyl substituted with one or more fluoro. In certain embodiments, R¹⁸ is fluoro phenyl, difluoro phenyl, or trifluoro phenyl. In certain embodiments, R¹⁸ is (trifluoromethyl) phenyl. In certain embodiments, R¹⁸ is para-(trifluoromethyl) phenyl. In certain embodiments, R¹⁸ is (fluoromethyl) phenyl or (difluoromethyl) phenyl. In certain embodiments, R¹⁸ is substituted phenyl and is more electron-withdrawing than unsubstituted phenyl. In certain embodiments, R¹⁸ is substituted phenyl and is not more electron-withdrawing than perfluorophenyl.

In certain embodiments, R¹⁸ is substituted or unsubstituted alkyl. In certain embodiments, R¹⁸ is unsubstituted C₁₋₆ alkyl or C₁₋₆ alkyl substituted with one or more fluoro. In certain embodiments, R¹⁸ is —CH₃. In certain embodiments, R¹⁸ is —CH₂F, —CHF₂, or —CF₃.

In certain embodiments, R¹⁹ is hydrogen. In certain embodiments, R¹⁹ is substituted or unsubstituted alkyl. In certain embodiments, R¹⁹ is unsubstituted C₁₋₆ alkyl or C₁₋₆ alkyl substituted with one or more fluoro. In certain embodiments, R¹⁹ is unsubstituted C₁₋₆ alkyl or C₁₋₆ alkyl substituted with one or more fluoro. In certain embodiments, R¹⁹ is —CH₃. In certain embodiments, R¹⁸ is —CH₂F, —CHF₂, or —CF₃.

In certain embodiments, the carbon atom to which R¹⁸ is attached is in the R configuration. In certain embodiments, the carbon atom to which R¹⁸ is attached is in the S configuration.

In certain embodiments, the compound is of the formula:

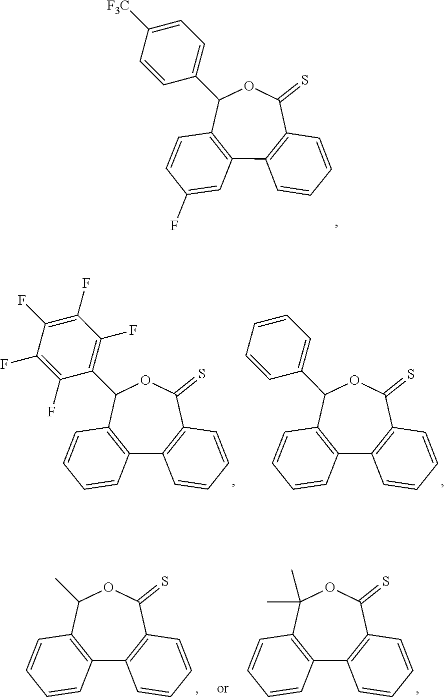

or a tautomer or salt thereof.

In certain embodiments, the compound is of the formula:

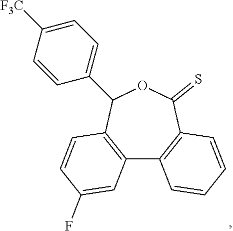 [SMILES: Fc1ccc2c(c1)-c1ccccc1C(=S)OC2c1ccc(C(F)(F)F)cc1]

or a tautomer or salt thereof.

In certain embodiments, the compound is of the formula:

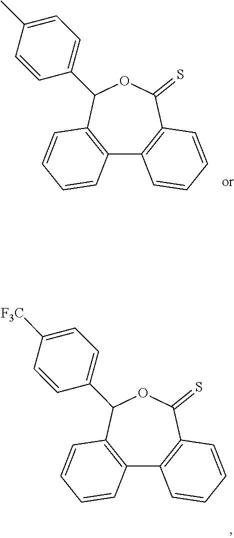 [SMILES: Cc1ccc(C2OC(=S)c3ccccc3-c3ccccc32)cc1 ; FC(F)(F)c1ccc(C2OC(=S)c3ccccc3-c3ccccc32)cc1]

or a tautomer or salt thereof.

In another aspect, the present disclosure provides a copolymer comprising:
m1 instances of a first type of repeating units of Formula i′:

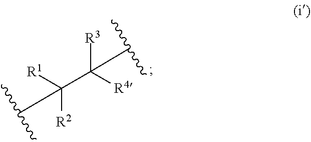

m2 instances of a second type of repeating units of Formula ii:

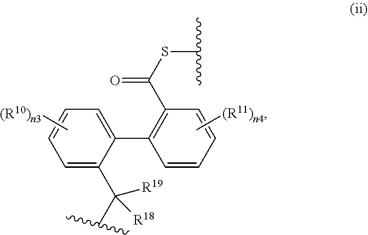

or a tautomer thereof;
optionally one or more types of crosslinking units; and
optionally one or more types of additional repeating units;
wherein:
   m1 is an integer between 10 and 1,000,000, inclusive;
   m2 is an integer between 2 and 1,000,000, inclusive;
   R¹, R², and R³ are each independently hydrogen, halogen, or substituted or unsubstituted alkyl;
   R⁴′ is —C(═O)OR^{a}, —C(═O)N(R^{a})₂, substituted or unsubstituted alkyl, halogen, substituted or unsubstituted carbocyclyl, substituted or unsubstituted heterocyclyl, substituted or unsubstituted aryl, substituted or unsubstituted heteroaryl, —CN, —OR^{a}, —SCN, —SR^{a}, —SSR^{a}, —N₃, —NO, —N(R^{a})₂, —NO₂, —C(═O)R^{a}, —C(═O)SR^{a}, —C(═NR^{a})R^{a}, —C(═NR^{a})OR^{a}, —C(═NR^{a})SR^{a}, —C(═NR^{a})N(R^{a})₂, —S(═O)R^{a}, —S(═O)OR^{a}, —S(═O)SR^{a}, —S(═O)N(R^{a})₂, —S(═O)₂R^{a}, —S(═O)₂OR^{a}, —S(═O)₂SR^{a}, —S(═O)₂N(R^{a})₂, —OC(═O)R^{a}, —OC(═O)OR^{a}, —OC(═O)SR^{a}, —OC(═O)N(R^{a})₂, —OC(═NR^{a})R^{a}, —OC(═NR^{a})OR^{a}, —OC(═NR^{a})SR^{a}, —OC(═NR^{a})N(R^{a})₂, —OS(═O)R^{a}, —OS(═O)OR^{a}, —OS(═O)SR^{a}, —OS(═O)N(R^{a})₂, —OS(═O)₂R^{a}, —OS(═O)₂OR^{a}, —OS(═O)₂SR^{a}, —OS(═O)₂N(R^{a})₂, —ON(R^{a})₂, —SC(═O)R^{a}, —SC(═O)OR^{a}, —SC(═O)SR^{a}, —SC(═O)N(R^{a})₂, —SC(═NR^{a})R^{a}, —SC(═NR^{a})OR^{a}, —SC(═NR^{a})SR^{a}, —SC(═NR^{a})N(R^{a})₂, —NR^{a}C(═O)R^{a}, —NR^{a}C(═O)OR^{a}, —NR^{a}C(═O)SR^{a}, —NR^{a}C(═O)N(R^{a})₂, —NR^{a}C(═NR^{a})R^{a}, —NR^{a}C(═NR^{a})OR^{a}, —NR^{a}C(═NR^{a})SR^{a}, —NR^{a}C(═NR^{a})N(R^{a})₂, —NR^{a}S(═O)R^{a}, —NR^{a}S(═O)OR^{a}, —NR^{a}S(═O)SR^{a}, —NR^{a}S(═O)N(R^{a})₂, —NR^{a}S(═O)₂R^{a}, —NR^{a}S(═O)₂OR^{a}, —NR^{a}S(═O)₂SR^{a}, —NR^{a}S(═O)₂N(R^{a})₂, —Si(R^{a})₃, —Si(R^{a})₂OR^{a}, —Si(R^{a})(OR^{a})₂, —Si(OR^{a})₃, —OSi(R^{a})₃, —OSi(R^{a})₂OR^{a}, —OSi(R^{a})(OR^{a})₂, or —OSi(OR^{a})₃;
each instance of R^{a} is independently hydrogen, substituted or unsubstituted alkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;
each instance of R¹⁰ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R¹¹ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R^{b} is independently hydrogen, substituted or unsubstituted alkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;
each of n3 and n4 is independently 0, 1, 2, 3, or 4;
R¹⁸ is substituted or unsubstituted aryl, substituted or unsubstituted alkyl, substituted or unsubstituted carbocyclyl, substituted or unsubstituted heterocyclyl, or substituted or unsubstituted heteroaryl; and
R¹⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
provided that no instance of R¹, R², R³, R⁴′, R¹⁰, R, R¹⁸, and R¹⁹ comprises one or more non-aromatic unsaturated CC bonds.

In another aspect, the present disclosure provides a copolymer prepared by a method comprising (e.g., consisting essentially of) polymerizing a first type of monomers, a second type of monomers, optionally one or more types of crosslinkers, and optionally one or more additional types of monomers, wherein:
the first type of monomers is of Formula I′:

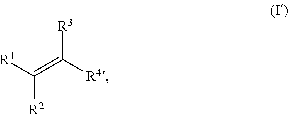

or a tautomer or salt thereof;
the second type of monomers is a compound provided herein, or a tautomer or salt thereof; and
each type of the crosslinkers, if present, is independently a small molecule comprising two or more non-aromatic unsaturated CC bonds.

In certain embodiments, m1 is an integer between 30 and 100, between 100 and 300, between 300 and 1000, between 1000 and 3000, between 3000 and 10000, inclusive. In certain embodiments, m1 is an integer between 300 and 3000, inclusive.

In certain embodiments, m2 is an integer between 3 and 10, between 10 and 30, between 30 and 100, between 100 and 300, between 300 and 1000, or between 1000 and 3000, inclusive. In certain embodiments, m2 is an integer between 30 and 300, inclusive.

In certain embodiments, m1:m2 is between 1:1 and 3:1, between 3:1 and 10:1, between 10:1 and 30:1, between 30:1 and 100:1, between 100:1 and 300:1, or between 300:1 and 1000:1, inclusive. In certain embodiments, m1:m2 is between 3:1 and 100:1, inclusive.

In certain embodiments, R¹ is hydrogen. In certain embodiments, R¹ is substituted or unsubstituted alkyl. In certain embodiments, R¹ is substituted or unsubstituted C₁₋₆ alkyl. In certain embodiments, R¹ is —CH₃.

In certain embodiments, R² is hydrogen. In certain embodiments, R² is substituted or unsubstituted alkyl. In certain embodiments, R² is substituted or unsubstituted C₁₋₆ alkyl. In certain embodiments, R² is —CH₃.

In certain embodiments, R³ is hydrogen. In certain embodiments, R³ is substituted or unsubstituted alkyl. In certain embodiments, R³ is substituted C₁-C₆ alkyl. In certain embodiments, R³ is unsubstituted C₁-C₆ alkyl. In certain embodiments, R³ is —CH₃.

In certain embodiments, each of R¹, R², and R³ is hydrogen.

In certain embodiments, R⁴′ is —C(═O)OR^{a}; wherein R^{a} is substituted or unsubstituted alkyl, substituted or unsubstituted carbocyclyl, substituted or unsubstituted heterocyclyl, substituted or unsubstituted aryl, or substituted or unsubstituted heteroaryl. In certain embodiments, R⁴′ is —C(═O)N(R^{a})₂; wherein each R^{a} is independently hydrogen, substituted or unsubstituted alkyl, substituted or unsubstituted carbocyclyl, substituted or unsubstituted heterocyclyl, substituted or unsubstituted aryl, or substituted or unsubstituted heteroaryl; provided that at least one R^{a} is not hydrogen. In certain embodiments, R⁴′ is —C(═O)OMe. In certain embodiments, R⁴′ is —C(═O)NMe₂. In certain embodiments, R⁴′ is —C(═O)O^{t}Bu, —C(═O)OBn, or —C(═O)OCH₂CH₂OMe.

In certain embodiments, the first type of repeating units is of the formula:

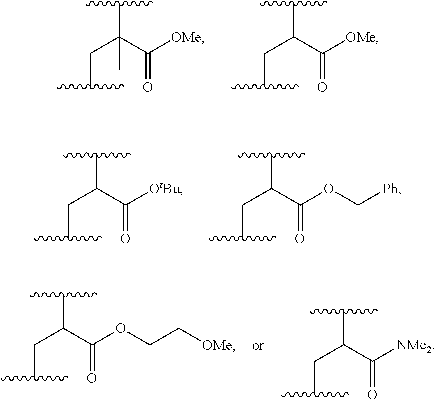

In certain embodiments, the first type of monomers is of the formula:

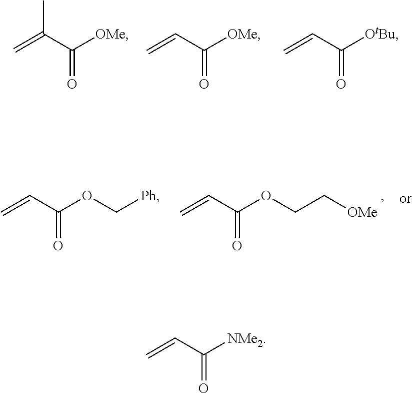

In certain embodiments, the first type of repeating units is of the formula:

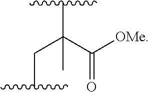

In certain embodiments, the first type of monomers is of the formula:

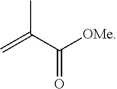

In certain embodiments, the second type of repeating units is of the formula:

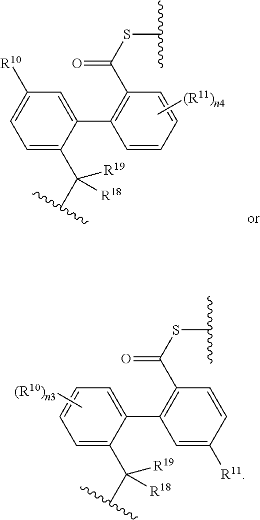

In certain embodiments, the second type of repeating units is of the formula:

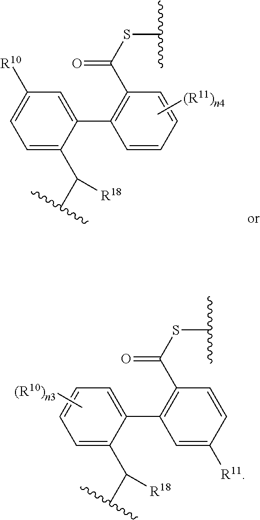

In certain embodiments, the second type of repeating units is of the formula:

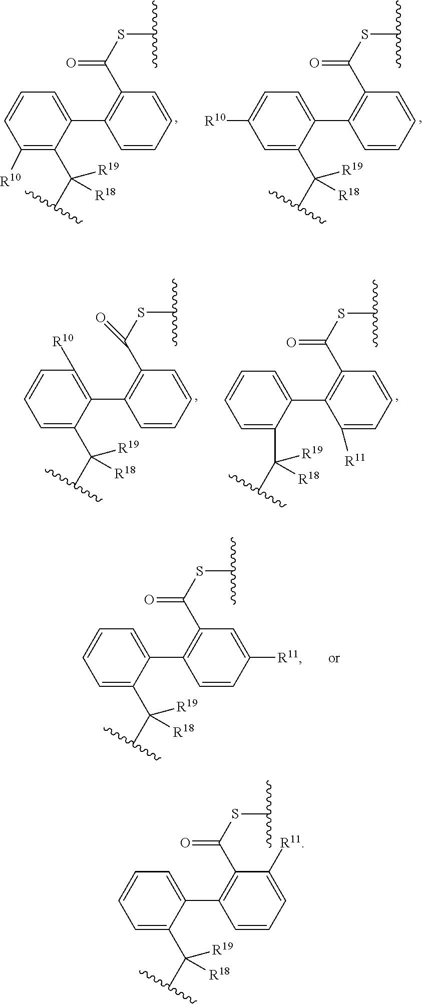

In certain embodiments, the second type of repeating units is of the formula:

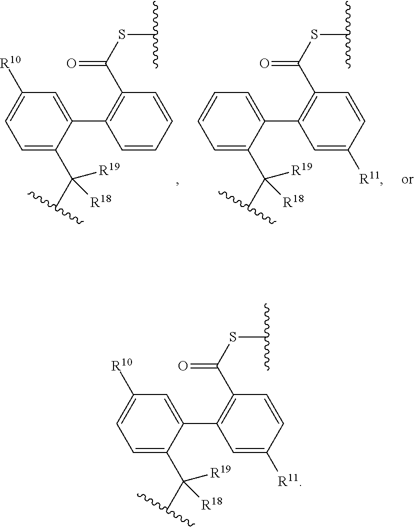

In certain embodiments, the second type of repeating units is of the formula:

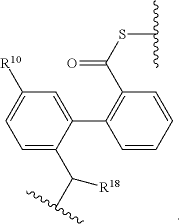

In certain embodiments, the second type of repeating units is of the formula:

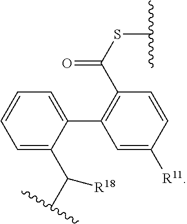

In certain embodiments, the second type of repeating units is of the formula:

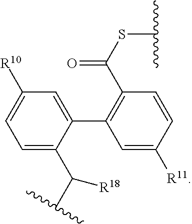

R¹⁰, n3, R¹¹, n4, R¹⁸, and R¹⁹ are as described herein.

In certain embodiments, n3 is 1. In certain embodiments, n3 is 0.

In certain embodiments, n4 is 0. In certain embodiments, n4 is 1.

In certain embodiments, at least one instance of R¹⁰ or R¹¹ is halogen, preferably, fluoro. In certain embodiments, at least one instance of R¹⁰ or R¹¹ is substituted or unsubstituted alkyl, —O(substituted or unsubstituted alkyl), or —S(substituted or unsubstituted alkyl). In certain embodiments, at least one instance of R¹⁰ or R¹¹ is unsubstituted C₁₋₆ alkyl, C₁₋₆ alkyl substituted with one or more fluoro, —O(unsubstituted C₁₋₆ alkyl), —O(C₁₋₆ alkyl substituted with one or more fluoro), —S(unsubstituted C₁₋₆ alkyl), or —S(C₁₋₆ alkyl substituted with one or more fluoro).

In certain embodiments, R¹⁸ is substituted or unsubstituted aryl. In certain embodiments, R¹⁸ is substituted or unsubstituted phenyl. In certain embodiments, R¹⁸ is unsubstituted phenyl or phenyl substituted with one or more: halogen, substituted or unsubstituted, C₁₋₆ alkyl, —OR^{c}, —SR^{c}, —N(R^{c})₂, —CN, —SCN, —NO₂, —C(═O)R^{c}, —C(═O)OR^{c}, —C(═O)N(R^{c})₂, —OC(═O)R^{c}, —OC(═O)OR^{c}, —OC(═O)N(R^{c})₂, —NR^{c}C(═O)R^{c}, —NR^{c}C(═O)OR^{c}, and/or —NR^{c}C(═O)N(R^{c})₂, wherein each instance of R^{c} is independently hydrogen or substituted or unsubstituted C₁₋₆ alkyl. In certain embodiments, R¹⁸ is unsubstituted phenyl or phenyl substituted with one or more: halogen, unsubstituted C₁₋₆ alkyl, and/or C₁₋₆ alkyl substituted with one or more fluoro. In certain embodiments, R¹⁸ is substituted or unsubstituted alkyl. In certain embodiments, R¹⁸ is unsubstituted C₁₋₆ alkyl or C₁₋₆ alkyl substituted with one or more fluoro.

In certain embodiments, R¹⁹ is hydrogen. In certain embodiments, R¹⁹ is substituted or unsubstituted alkyl. In certain embodiments, R¹⁹ is unsubstituted C₁₋₆ alkyl or C₁₋₆ alkyl substituted with one or more fluoro.

In certain embodiments, the second type of repeating units is of the formula:

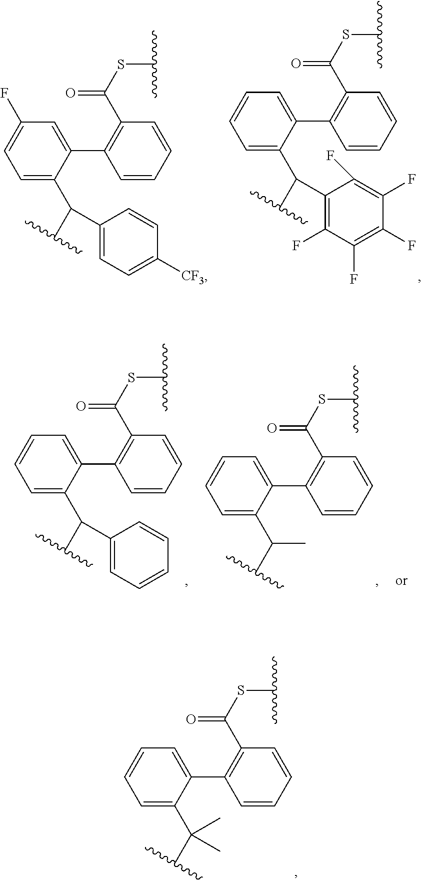

or a tautomer thereof.

In certain embodiments, the second type of repeating units is of the formula:

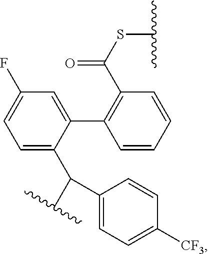

or a tautomer thereof.

In certain embodiments, the second type of repeating units is of the formula:

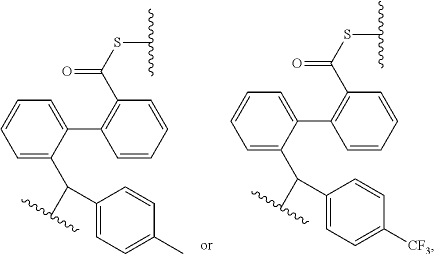

or a tautomer thereof.

In certain embodiments, the method of polymerizing comprises substantially no crosslinkers.

In certain embodiments, the method of polymerizing comprises substantially no additional types of monomers.

In certain embodiments, at least one additional type of monomers is an end-group-forming monomer. In certain embodiments, at least one additional type of monomers is a chain transfer agent. In certain embodiments, at least one additional type of monomers is a dithioate (e.g., (substituted or unsubstituted, C₁₋₆ alkyl)-S—C(═S)-(substituted or unsubstituted phenyl)).

In certain embodiments, the molar ratio of the first type of repeating units to all additional type of repeating units or the molar ratio of the first type of monomers to all additional types of monomers is between 3:1 and 10:1, between 10:1 and 30:1, between 30:1 and 100:1, between 100:1 and 300:1, between 300:1 and 1000:1, inclusive.

In certain embodiments, at least one type of the crosslinking units is of the formula:

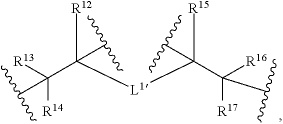

wherein:
   each instance of L¹′ is independently substituted or unsubstituted, C₁₋₁₀₀ alkylene or substituted or unsubstituted, C₂₋₁₀₀ heteroalkylene, optionally wherein one or more backbone carbon atoms of the C₁₋₁₀₀ alkylene and/or C₂₋₁₀₀ heteroalkylene are independently replaced with substituted or unsubstituted carbocyclylene, substituted or unsubstituted heterocyclylene, substituted or unsubstituted arylene, or substituted or unsubstituted heteroarylene; and
   each instance of R¹², R¹³, R¹⁴, R¹⁵, R¹⁶, and R¹⁷ is independently hydrogen, halogen, or substituted or unsubstituted alkyl;
   provided that no instance of L¹′, R¹², R¹³, R¹⁴, R¹⁵, R¹⁶, and R¹⁷ comprises one or more non-aromatic unsaturated CC bonds.

In certain embodiments, at least one type of the crosslinkers is of the formula:

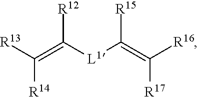

or a tautomer or salt thereof, wherein:
   each instance of L¹′ is independently substituted or unsubstituted, C₁₋₁₀₀ alkylene or substituted or unsubstituted, C₂₋₁₀₀ heteroalkylene, optionally wherein one or more backbone carbon atoms of the C₁₋₁₀₀ alkylene and/or C₂₋₁₀₀ heteroalkylene are independently replaced with substituted or unsubstituted carbocyclylene, substituted or unsubstituted heterocyclylene, substituted or unsubstituted arylene, or substituted or unsubstituted heteroarylene; and
   each instance of R¹², R¹³, R¹⁴, R¹⁵, R¹⁶, and R¹⁷ is independently hydrogen, halogen, or substituted or unsubstituted alkyl;
   provided that no instance of L¹′, R¹², R¹³, R¹⁴, R¹⁵, R¹⁶, and R¹⁷ comprises one or more non-aromatic unsaturated CC bonds.

In certain embodiments, at least one instance of L¹′ is independently substituted or unsubstituted, C₁₋₁₀₀ alkylene or substituted or unsubstituted, C₂₋₁₀₀ heteroalkylene. In certain embodiments, at least one instance of L¹′ is substituted or unsubstituted, C₂₋₁₂ heteroalkylene. In certain embodiments, at least one instance of L¹′ is unsubstituted C₂₋₁₂ heteroalkylene or C₂₋₁₂ heteroalkylene substituted with one or more: oxo, unsubstituted C₁₋₆ alkyl, and/or C₁₋₆ alkyl substituted with one or more fluoro. In certain embodiments, at least one instance of L¹′ is —C(═O)—O—(CH₂)ₚ₁—O—C(═O)—, wherein each instance of p1 is independently 2, 3, 4, 5, or 6.

In certain embodiments, each instance of R¹², R¹³, R¹⁴, R¹⁵, R¹⁶, and R¹⁷ is independently hydrogen or substituted or unsubstituted, C₁₋₆ alkyl. In certain embodiments, each instance of R¹², R¹³, R¹⁴, R¹⁵, R¹⁶, and R¹⁷ is independently hydrogen or unsubstituted C₁₋₃ alkyl. In certain embodiments, each instance of R¹², R¹³, R¹⁴, R¹⁵, R¹⁶, and R¹⁷ is independently hydrogen or —CH₃. In certain embodiments, each instance of R¹², R¹³, R¹⁴, R¹⁵, R¹⁶, and R¹⁷ is hydrogen.

In certain embodiments, no instance of R¹, R², R³, R⁴′, R¹⁰, R¹¹, R¹⁸, and R¹⁹ comprises one or more non-aromatic CC double bonds. In certain embodiments, no instance of R¹, R², R³, R⁴′, R¹⁰, R¹¹, R¹⁸, and R¹⁹ comprises one or more non-aromatic CC triple bonds.

In certain embodiments, the copolymer is substantially uncrosslinked.

In certain embodiments, the molar ratio of the first type of repeating units to all types of crosslinking units or the molar ratio of the first type of monomers to all types of crosslinkers is between 2:1 and 10:1, between 10:1 and 30:1, or between 30:1 and 100:1, inclusive.

In certain embodiments, the crosslinking degree of the copolymer is between 0.1% and 0.3%, between 0.3% and 1%, between 1% and 3%, between 3% and 10%, between 10% and 20%, or between 20% and 50%, inclusive, mole:mole. In certain embodiments, the crosslinking degree of the copolymer is not more than 0.1%, not more than 1%, or not more than 10%, inclusive, mole:mole. In certain embodiments, the crosslinking degree is determined by the consumption of the monomers that are polymerized to form the copolymer. In certain embodiments, the crosslinking degree is determined by the consumption of the comonomers and crosslinkers that are polymerized to form the copolymer.

In certain embodiments, the number-average molecular weight of the copolymer as determined by gel permeation chromatography is between 1 kDa and 3 kDa, between 3 kDa and 10 kDa, between 10 kDa and 30 kDa, between 30 kDa and 100 kDa, between 100 kDa and 300 kDa, between 300 kDa and 1,000 kDa, between 1,000 kDa and 3,000 kDa, or between 3,000 kDa and 10,000 kDa.

In certain embodiments, the number-average molecular weight of the copolymer as determined by gel permeation chromatography is between 2 kDa and 3 kDa, between 3 kDa and 4 kDa, between 4 kDa and 6 kDa, between 6 kDa and 8 kDa, or between 8 kDa and 10 kDa.

In certain embodiments, the number-average molecular weight of the copolymer as determined by gel permeation chromatography is between 100 kDa and 300 kDa, between 300 kDa and 1,000 kDa, between 1,000 kDa and 3,000 kDa, or between 3,000 kDa and 10,000 kDa.

In certain embodiments, the number-average molecular weight of the copolymer as determined by size exclusion chromatography is between 1 kDa and 3 kDa, between 3 kDa and 10 kDa, between 10 kDa and 30 kDa, between 30 kDa and 100 kDa, between 100 kDa and 300 kDa, between 300 kDa and 1,000 kDa, between 1,000 kDa and 3,000 kDa, or between 3,000 kDa and 10,000 kDa. In certain embodiments, the number-average molecular weight of the copolymer as determined by gel size exclusion chromatography is between 2 kDa and 3 kDa, between 3 kDa and 4 kDa, between 4 kDa and 6 kDa, between 6 kDa and 8 kDa, or between 8 kDa and 10 kDa.

In certain embodiments, the number-average molecular weight of the copolymer as determined by size exclusion chromatography is between 100 kDa and 300 kDa, between 300 kDa and 1,000 kDa, between 1,000 kDa and 3,000 kDa, or between 3,000 kDa and 10,000 kDa.

In certain embodiments, the copolymer is a random copolymer. In certain embodiments, the copolymer is a statistical copolymer. In certain embodiments, the copolymer is a block copolymer.

In certain embodiments, the copolymer is degradable after reacting with a nucleophile.

In another aspect, the present disclosure provides a composition comprising:
a compound provided herein, or a tautomer or salt thereof; and
optionally an excipient.

In certain embodiments, the excipient is one single excipient. In certain embodiments, the excipient is a mixture of two or more (e.g., three) excipients. In certain embodiments, the excipient is a solvent described herein.

In another aspect, the present disclosure provides a composition comprising:
a copolymer provided herein; and
optionally an excipient.

In another aspect, the present disclosure provides a kit comprising:
the compound, or a tautomer or salt thereof, or the composition; and
instructions for using the compound, tautomer, salt, or composition.

In another aspect, the present disclosure provides a kit comprising:
the copolymer or the composition; and
instructions for using the copolymer or composition.

In another aspect, the present disclosure provides a method of preparing the copolymer comprising (e.g., consisting essentially of) polymerizing the first type of monomers, the second type of monomers, optionally the crosslinkers, and optionally the additional types of monomers.

In certain embodiments, the step of polymerizing is polymerizing by a radical polymerization reaction.

In certain embodiments, the molar ratio of the first type of repeating units to the second type of repeating units, or the molar ratio of the first type of monomers to the second type of monomers is between 1:0.01 and 1:0.03, between 1:0.03 and 1:0.1, between 1:0.1 and 1:0.3, between 1:0.3 and 1:0.5, between 1:0.5 and 1:0.6, or between 1:0.6 and 1:0.7, inclusive. In certain embodiments, the molar ratio of the first type of monomers to the second type of monomers is between 1:0.05 and 1:0.12, inclusive. In certain embodiments, the molar ratio of the first type of repeating units to the second type of repeating units, or the molar ratio of the first type of monomers to the second type of monomers is between 1:0.025 and 1:0.25, inclusive.

In certain embodiments, the step of polymerizing further comprises a radical initiator. In certain embodiments, the radical initiator is substantially one single radical initiator. In certain embodiments, the radical initiator is a mixture of two or more (e.g., three) radical initiators. In certain embodiments, the radical initiator is an azo compound, organic peroxide, or inorganic peroxide. In certain embodiments, the radical initiator is 2,2′-azobis(2-methylpropionitrile) (AIBN), 1,1′-diazene-1,2-diyldicyclohexanecarbonitrile (ACHN), di-tert-butyl peroxide, benzoyl peroxide, methyl ethyl ketone peroxide, acetone peroxide, or peroxydisulfate salt. In certain embodiments, the radical initiator is dichlorine.

In certain embodiments, the molar ratio of the radical initiator to the first type of monomers is between 0.0001:1 and 0.0003:1, between 0.0003:1 and 0.001:1, between 0.001:1 and 0.003:1, or between 0.003:1 and 0.01:1, inclusive.

In certain embodiments, the step of polymerizing further comprises a solvent. In certain embodiments, the solvent is substantially one single solvent. In certain embodiments, the solvent is a mixture of two or more (e.g., three) solvents (e.g., solvents described in this paragraph). In certain embodiments, the solvent is an organic solvent. In certain embodiments, the solvent is an aprotic solvent. In certain embodiments, the solvent is an ether solvent. In certain embodiments, the solvent is a ketone solvent. In certain embodiments, the solvent is an alkane solvent. In certain embodiments, the solvent is an aromatic organic solvent. In certain embodiments, the solvent is benzene, toluene, o-xylene, m-xylene, or p-xylene, or a mixture thereof. In certain embodiments, the solvent is toluene. In certain embodiments, the solvent is a non-aromatic organic solvent. In certain embodiments, the solvent is acetonitrile, dioxane, or tetrahydrofuran, or a mixture thereof. In certain embodiments, the solvent is acetonitrile. In certain embodiments, the solvent is acetone, chloroform, dichloromethane, diethyl ether, ethyl acetate, methyl tert-butyl ether, or 2-methyltetrahydrofuran, or a mixture thereof. In certain embodiments, the solvent is dimethyl sulfoxide, N,N-dimethylformamide, N,N-dimethylacetamide, or a mixture thereof. In certain embodiments, the boiling point of the solvent at about 1 atm is between 30 and 50, between 50 and 70, between 70 and 100, between 100 and 130, between 130 and 160, or between 16° and 200° C., inclusive.

In certain embodiments, the step of polymerizing is substantially free of a solvent.

In certain embodiments, the temperature of the step of polymerizing is between 20 and 40, between 40 and 60, between 60 and 90, between 90 and 120, or between 12° and 150° C., inclusive. In certain embodiments, the temperature of the step of polymerizing is between 8° and 120° C., inclusive. In certain embodiments, the temperature of the step of polymerizing is substantially constant over the time duration of the step of polymerizing. In certain embodiments, the temperature of the step of polymerizing is a variable temperature (e.g., ±5, ±10, ±15, or ±20° C.) over the time duration of the step of polymerizing.

In certain embodiments, the time duration of the step of polymerizing is between 1 and 3 hours, between 3 and 8 hours, between 8 and 24 hours, between 1 and 3 days, or between 3 and 7 days, inclusive. In certain embodiments, the time duration of the step of polymerizing is between 2 and 24 hours, inclusive.

In another aspect, the present disclosure provides a method of degrading the copolymer comprising reacting the copolymer with a nucleophile.

In certain embodiments, immediately after the step of degrading, between 10% and 20%, between 20% and 30%, between 30% and 50%, between 50% and 70%, or between 70% and 90% of the C—S bonds in the backbone of the copolymer is cleaved.

In certain embodiments, the step of reacting the copolymer with the nucleophile comprises a second time duration. In certain embodiments, the second time duration is between 10 minute and 1 hour, between 1 and 8 hours, between 8 and 24 hours, between 1 and 3 days, or between 3 and 7 days, inclusive. In certain embodiments, the second time duration is between 8 hours and 3 days.

In certain embodiments, the nucleophile degrades the copolymer under ambient conditions. In certain embodiments, the step of reacting the copolymer with the nucleophile comprises a second temperature. In certain embodiments, the second temperature is ambient temperature. In certain embodiments, the second temperature is between 20 and 40, between 40 and 60, between 60 and 80, between 80 and 100, or between 10° and 120° C., inclusive. In certain embodiments, the second temperature is between 4° and 60° C., inclusive. In certain embodiments, the second temperature is substantially constant over the second time duration. In certain embodiments, the second temperature is a variable temperature (e.g., ±5, ±10, ±15, or ±20° C.) over the second time duration.

In certain embodiments, the nucleophile is an amine. In certain embodiments, the nucleophile is an aliphatic amine. In certain embodiments, the nucleophile is aromatic amine. In certain embodiments, the nucleophile is (substituted or unsubstituted alkyl)-NH₂, preferably (unsubstituted C₂₋₆ alkyl)-NH₂. In certain embodiments, the nucleophile is n-propylamine. In certain embodiments, the nucleophile is (substituted or unsubstituted alkyl)₂NH or (substituted or unsubstituted alkyl)₃N. In certain embodiments, the nucleophile is (substituted or unsubstituted, C₁₋₆ alkyl)₂NH or (substituted or unsubstituted, C₁₋₆ alkyl)₃N. In certain embodiments, the nucleophile is (alkyl substituted at least with —SH)—NH₂, preferably HS—(CH₂)₂₋₆—NH₂. In certain embodiments, the nucleophile is an amidine (e.g., 1,8-diazabicyclo[5.4.0]undec-7-ene (DBU), diminazene, benzamidine, pentamidine, or paranyline). In certain embodiments, the nucleophile is a primary amine. In certain embodiments, the nucleophile is methylamine, ethylamine, n-propylamine, isopropylamine, n-butylamine, n-pentylamine, n-hexylamine, cyclohexylamine, ethanolamine (i.e., 2-aminoethanol), tris (i.e., 2-amino-2-hydroxymethyl-propane-1,3-diol), ethylenediamine, triethylenediamine, or aniline. In certain embodiments, the nucleophile is a secondary amine. In certain embodiments, the nucleophile is dimethylamine, diethylamine, di-n-propylamine, diisopropylamine, ethylisopropylamine, dicyclohexylamine, methylethanolamine, pyrrolidine, piperidine, morpholine, piperazine, or 1,4-bis-(3-aminopropyl)piperazine. In certain embodiments, the nucleophile is a tertiary amine. In certain embodiments, the nucleophile is trimethylamine, triethylamine, diisopropylethylamine (DIPEA), tri-n-butylamine, 4-dimethylaminopyridine (DMAP), or 1,8-diazabicyclo[5.4.0]undec-7-ene (DBU). In certain embodiments, the nucleophile is a thiol. In certain embodiments, the nucleophile is (substituted or unsubstituted alkyl)-SH, preferably (unsubstituted C₂₋₆ alkyl)-SH.

In certain embodiments, the nucleophile is substantially one compound. In certain embodiments, the nucleophile is a mixture of two or more (e.g., three) compounds. In certain embodiments, the nucleophile is a mixture of two or more amines. In certain embodiments, the nucleophile is a mixture of (1) (substituted or unsubstituted, C₁₋₆ alkyl)-NH₂, (substituted or unsubstituted, C₁₋₆ alkyl)₂NH, or (substituted or unsubstituted, C₁₋₆ alkyl)₃N (e.g., at between 80% and 99%, v/v, inclusive); and an amidine (e.g., DBU) (e.g., at between 1% and 20%, v/v, inclusive).

In certain embodiments, the step of reacting the copolymer with the nucleophile comprises no solvent.

In certain embodiments, the step of reacting the copolymer with the nucleophile comprises a second solvent. In certain embodiments, the second solvent is substantially one single solvent. In certain embodiments, the second solvent is a mixture of two or more (e.g., three) solvents (e.g., solvents described in this paragraph). In certain embodiments, the second solvent is an organic solvent. In certain embodiments, the second solvent is an aprotic solvent. In certain embodiments, the second solvent is an ether solvent. In certain embodiments, the second solvent is a ketone solvent. In certain embodiments, the second solvent is an alkane solvent. In certain embodiments, the second solvent is an alcohol solvent. In certain embodiments, the second solvent is an aromatic organic solvent. In certain embodiments, the second solvent is benzene, toluene, o-xylene, m-xylene, or p-xylene, or a mixture thereof. In certain embodiments, the second solvent is a non-aromatic organic solvent. In certain embodiments, the second solvent is acetonitrile, dioxane, or tetrahydrofuran, or a mixture thereof. In certain embodiments, the second solvent is acetonitrile. In certain embodiments, the second solvent is acetone, chloroform, dichloromethane, diethyl ether, ethyl acetate, methyl tert-butyl ether, or 2-methyltetrahydrofuran, or a mixture thereof. In certain embodiments, the second solvent is dimethyl sulfoxide, N,N-dimethylformamide, N,N-dimethylacetamide, or a mixture thereof. In certain embodiments, the second solvent is an inorganic solvent (e.g., water). In certain embodiments, the boiling point of the second solvent at about 1 atm is between 30 and 50, between 50 and 70, between 70 and 100, between 100 and 130, between 130 and 160, or between 16° and 200° C., inclusive.

In certain embodiments, a step of a method described herein is under a pressure between 0.5 and 1.1 atm (e.g., between 0.8 and 1.1 atm), inclusive.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1. Mechanism-Guided Discovery of Cleavable Comonomers for Backbone Deconstructable Polymethylmethacrylate (PMMA)

This example describes the design of a class of benzyl-functionalized thionolactones (bDOTs). Guided by detailed mechanistic analyses, the introduction of radical-stabilizing substituents to bDOTs was shown to enable markedly increased and tunable copolymerization reactivity with methyl methacrylate (MMA). Through iterative optimizations of molecular structure, a specific bDOT, F-p-CF₃PhDOT, was discovered to copolymerize with MMA to achieve a nearly even distribution within the resulting copolymers. High molecular weight deconstructable PMMA (dPMMA, Mₙ>120 kDa) with low percentages of F-p-CF₃PhDOT (1.8 and 3.8%) were prepared using industrially relevant bulk free radical copolymerization. The thermomechanical properties dPMMA are similar to PMMA; however, the former were shown to degrade into low-molecular-weight fragments (<6.5 kDa) under mild aminolysis conditions. This example presents a radical ring-opening CC capable of nearly random copolymerization with MMA without crosslinking.

High-performance polymers often pose end-of-life management challenges. Integrating chemical deconstructability into these polymers is advantageous, as it not only facilitates their degradation but also provides opportunities for recycling and upcycling through further chemical processing of deconstructed fragments. This integration can be achieved through the cleavable comonomer (CC) approach, in which small molecules known as CCs are incorporated into the polymer backbone via copolymerization, introducing functionalities that are cleavable by external triggers.¹⁻⁷ For an effective implementation, the CC needs to be compatible with existing industrial polymerization methods, allowing for its rapid incorporation into current manufacturing processes. In addition, the CC loading needs to be minimal to avoid compromising the desirable properties of the original material and to minimize the impact on cost while still enabling maximal deconstruction. Therefore, an optimal CC should exhibit favorable copolymerization reactivity with the monomer and polymerization reaction of interest, ensuring the formation of the smallest possible degraded fragments at low loadings.

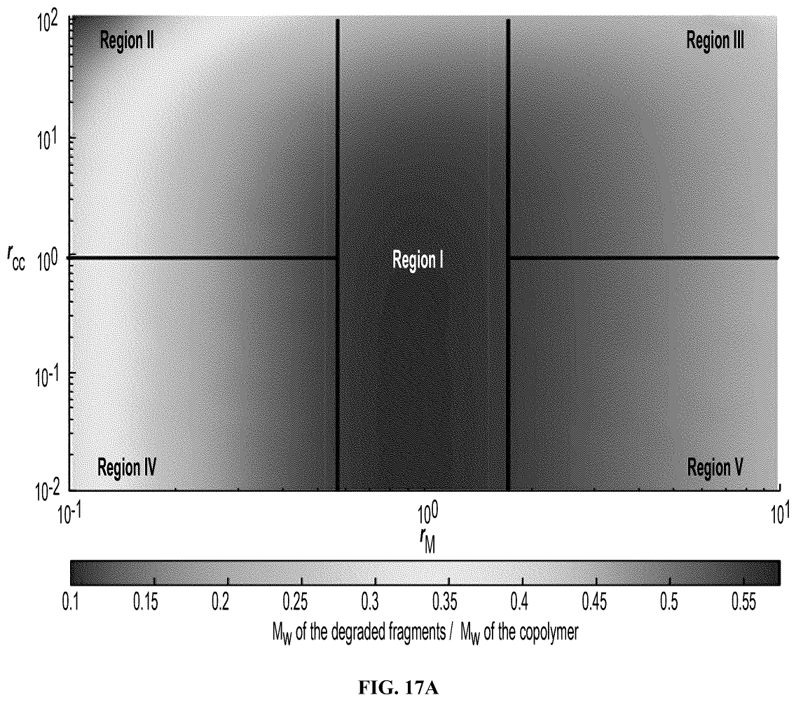

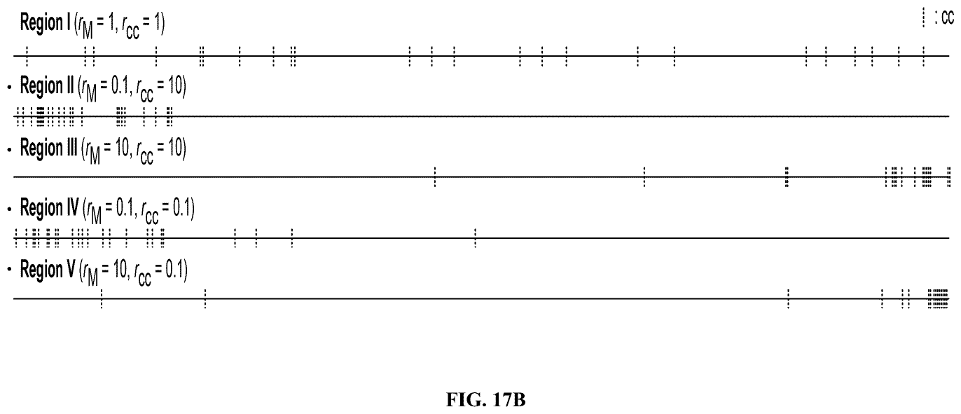

In chain-growth polymers, the copolymerization behavior of a CC with a target monomer (M) is governed by two reactivity ratios based on terminal models:

$${r}_{M} = \frac{{k}_{M to M}}{{k}_{M to CC}} , {r}_{CC} = \frac{{k}_{CC to CC}}{{k}_{CC to M}}$$

where k_{A to B} represents the rate constant for propagation from a chain ending in monomer A to monomer B (A and B each being either M or CC).⁸ When both reactivity ratios are >1 or <1, blocky or alternating copolymers result, respectively. A random comonomer distribution occurs when both r_{M} and r_{CC} are exactly equal to one. Gradient structures (in controlled radical polymerizations) or compositional drift (in free-radical polymerizations) emerge when one reactivity ratio is >1 and the other <1, with the monomer having the larger reactivity ratio being consumed first. Previous studies have used Monte Carlo simulations to examine copolymer sequences based on reactivity ratios.⁹⁻¹⁵ This methodology can be extended to model fragment sizes post degradation in the context of the CC approach.¹⁶⁻¹⁸ For example, simulations were conducted involving 2.5% CC loading with a degree of polymerization (DP) of 1,000 and plotted the weight-average molecular weight (M_{w}) of the oligomeric fragments obtained after CC cleavage relative to the M_{w} of the starting copolymer under various reactivity ratio scenarios (FIG. 17A; see Simulation Details section for further information). The use of M_{w} for the plotting conveys information about the largest fragments, which is important for assessing the performance of a CC. As shown in FIG. 17A, the fragment size remains largely unaffected by r_{CC} across a 0.01-100 range due to the low CC loading of 2.5%. Fluctuations in r_{M}, e.g., ranging from 0.1 to 10, may have a substantial impact. The size reaches its minimum when r_{M} is equal to one, indicating that an optimal CC requires an r_{M} value close to one for the most efficient deconstruction. Examples of simulated polymer sequences corresponding to different regions of FIG. 17A are illustrated in FIG. 17B. A deviation from the ideal (Region I) may produce a notable composition gradient in the copolymer structure (Region II-V), which results in inefficient deconstruction. These findings suggest the importance of reactivity ratios in the development of CCs and provide guidance for the preferred values: r_{M} being as close as possible to 1, while r_{CC} being less significance if it falls within the range of 0.01-100.

Vinyl polymers, which account for half of all polymers produced, are limited in their deconstructability because of their carbon-carbon backbones, which has driven the development of CCs¹⁹⁻²¹ or post-polymerization methods²²⁻²⁵ to install cleavable bonds. Because a large fraction of vinyl polymers are synthesized industrially through radical polymerization, various classes of CCs that are compatible with this process have been developed via three main approaches: 1) copolymerizing dioxygen to introduce cleavable peroxy bonds²⁶⁻³⁸; 2) using comonomers with functional groups that can generate radicals in the polymer backbone to trigger deconstruction³⁹⁻⁴⁵; and 3) employing comonomers capable of radical ring-opening polymerization (rROP) to introduce cleavable functionality.^{46,47} Among these approaches, rROP CCs have been extensively studied as they offer molecular tunability and enable a variety of deconstruction mechanisms.

Despite recent advances in rROP chemistry, formulating efficient rROP CCs for methacrylates remains challenging. Cyclic ketene acetals (CKAs)⁴⁸⁻⁵⁰ and sulfide-based cyclic methacrylates (SCMs)⁵¹⁻⁶⁰ are examples of rROP CCs, and they have been explored for their ability to copolymerize with methacrylic monomers⁶¹⁻⁷⁶ (FIG. 17B, I and II). Copolymerizations of methacrylates with CKAs exhibit pronounced compositional gradients (r_{M} significantly exceeds one), while copolymerizations with SCMs display improved reactivity but can lead to the formation of unwanted crosslinks. Moreover, these CCs introduce ester functionalities into the polymer backbone, the cleavage of which can compromise the ester functional groups in the methacrylate monomers unless a weaker bond is deliberately incorporated into the CC.^{51,56} Recently, thionolactones (TLs) have emerged as a type of rROP CC for enabling the installation of thioester linkages into polymer backbones, the latter of which may be efficiently and selectively cleaved under comparatively mild conditions (FIG. 17B, III).⁷⁷ TLs have proven successful as CCs for various vinyl polymers. An example is the TL molecule dibenzo[c,e]oxepine-5(7H)-thione, commonly referred to as “DOT,” which has demonstrated copolymerization reactivity with various acrylate,^{5, 78-86} acrylamide,^{78,80,81} and styrenic^{4, 82-85, 87} monomers; however, there have been no successful instances of TLs copolymerizing with methacrylates without exhibiting negligible reactivity or pronounced compositional gradients,⁸⁸ which can be attributed to their reactivity ratios being far from optimal.

To develop a TL optimized for methacrylates, the potential energy landscape of propagation was investigated, as reactivity ratios are intrinsically associated with the kinetics of propagation. In particular, development of a CC for methyl methacrylate (MMA) was pursued, as this is a widely used type of methacrylate, and it has broad applications ranging from transparent glass substitutes to automotive components and surface coatings.^{89,90} This example details the mechanism-driven development of a new class of CCs, termed benzylic-functionalized DOTs, or “bDOTs” (FIG. 17B, IV), which led to the discovery of an efficient CC, F-p-CF₃PhDOT, designed for use with the commodity polymer polymethylmethacrylate (PMMA). This example demonstrates the excellent compatibility of F-p-CF₃PhDOT with bulk free-radical polymerization (FRP) methods, which are standard in the production of high molecular weight industrial PMMA. Moreover, the favorable reactivity ratios of F-p-CF₃PhDOT enable efficient deconstruction of PMMA without compromising material properties, thereby confirming its effectiveness as a CC. F-p-CF₃PhDOT is capable of introducing well-distributed thioester functionalities into polymethacrylates, thus facilitating efficient degradation at low CC loadings.

DFT calculations enable the mechanism-guided design of bDOTs.

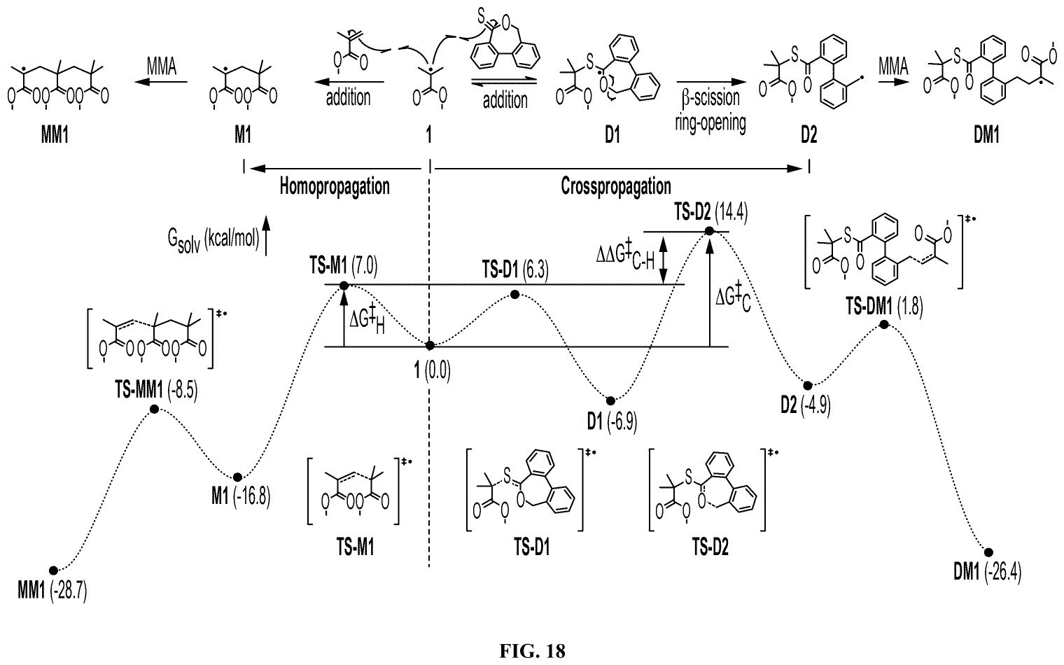

A mechanistic investigation was performed to on the inability of DOT to copolymerize with MMA under radical polymerization conditions. As discussed, the copolymerization behavior of DOT and MMA may be governed by two reactivity ratios:

$${r}_{MMA} = \frac{{k}_{MMA to MMA}}{{k}_{MMA to DOT}} , {r}_{DOT} = \frac{{k}_{DOT to DOT}}{{k}_{DOT to MMA}}$$

where k_{A to B} represents the rate constant for propagation from a chain ending in monomer A to monomer B (A and B being MMA or DOT). A previous report indicated that DOT acts as a spectator during copolymerization with MMA, with MMA undergoing homopolymerization.⁷⁸ Thus, a propagating chain ending in MMA homopropagates considerably faster than it cross-propagates, which would correspond to a large r_{MMA}. Further, as the simulations show (FIG. 17A), r_{MMA} would have a more dominant influence on the copolymerization behavior than r_{DOT} in circumstances where a minimal amount of DOT is added to the reaction mixture as a CC. Accordingly, density functional theory (DFT) calculations were employed to model the competition between homopropagation and cross-propagation of a propagating chain ending in MMA (FIG. 18). To reduce the computational cost, the homopropagation was simplified as radical 1 adding to MMA, and the cross-propagation was modeled as 1 adding to DOT, followed by β-scission and ring-opening, ultimately leading to the incorporation of DOT. This is an example where a computational tool was employed to analyze the copolymerization between TLs and methacrylates. Guillaneuf and co-workers⁸⁵ recently reported on the use of DFT calculations to aid in the development of a TL with improved reactivity for styrene and acrylate monomers. Their approach involved defining a composite rate constant that combines the multi-step process of radical addition to TLs, which may be beneficial for evaluating and predicting the reactivity of CCs when there are known molecules that copolymerize with the monomer of interest as reference points, as is the case with styrene and acrylates. By contrast, the present example involves analyzing the entire radical addition potential energy landscape and identifying key transition states that determine the reactivity ratios, which enables the discovery of a CC tailored for a challenging target monomer where no current solutions exist (e.g., MMA) and provides detailed mechanistic understanding of the impacts of CC molecular modification on the energy profile of rROP.

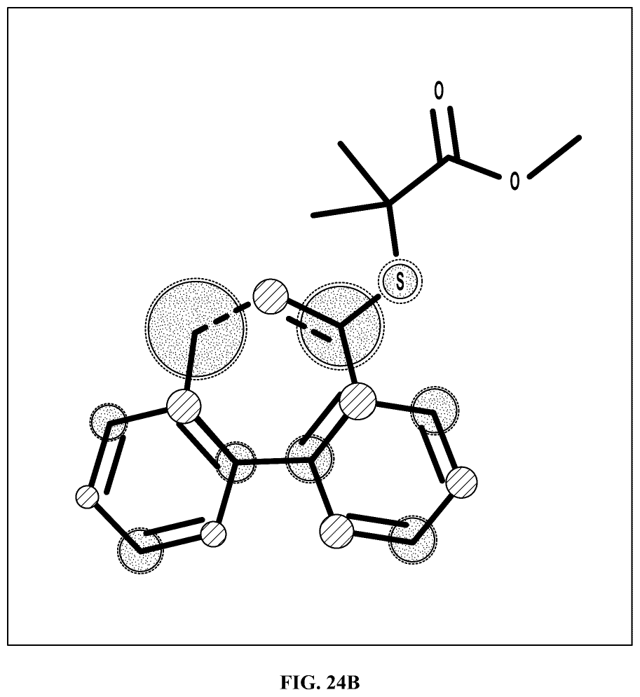

The DFT calculations described herein suggested that the rate-determining step for the cross-propagation of a chain ending with MMA may be the ring-opening of DOT, as evidenced by its calculated transition state (TS-D1) energy being 8.1 kcal/mol higher than that for the addition step (TS-D2). Although the kinetic barrier for cross-propagation is thermally accessible (ΔG^{‡}_{C}=14.4 kcal/mol), it is 7.4 kcal/mol higher than that for homopropagation (ΔG^{‡}_{H}=7.0 kcal/mol). This finding aligns with experimental observations where DOT acts as a spectator during MMA homopolymerization. The propagations leading to the formation of M1 and D2 are deemed irreversible at an appreciable concentration of MMA, as the subsequent monomer addition to form MM1 and DM1, respectively, is kinetically more favorable by >12 kcal/mol than the reverse reaction regenerating 1 and D1. Consequently, monomer addition to propagating chains ending in MMA is likely to be under Curtin-Hammett control, with the energy difference between TS-M1 and TS-D2 (ΔΔG^{‡}_{C-H}) dictating the reactivity ratio r_{MMA}. Thus, lowering the energy of TS-D2 to match that of TS-M1, such that r_{MMA} nears the optimal value of one as shown in FIG. 17A, may be important for achieving the copolymerization of DOT and MMA. Mulliken spin population analysis of the rate-determining transition state, TS-D2, showed that the benzylic carbon of DOT carries the highest spin population of 0.489 (FIGS. 24A-24B), which suggests that introducing radical-stabilizing substituent(s) onto the benzylic carbon of DOT might enable the copolymerization of DOT and MMA by lowering the energy of TS-D2.

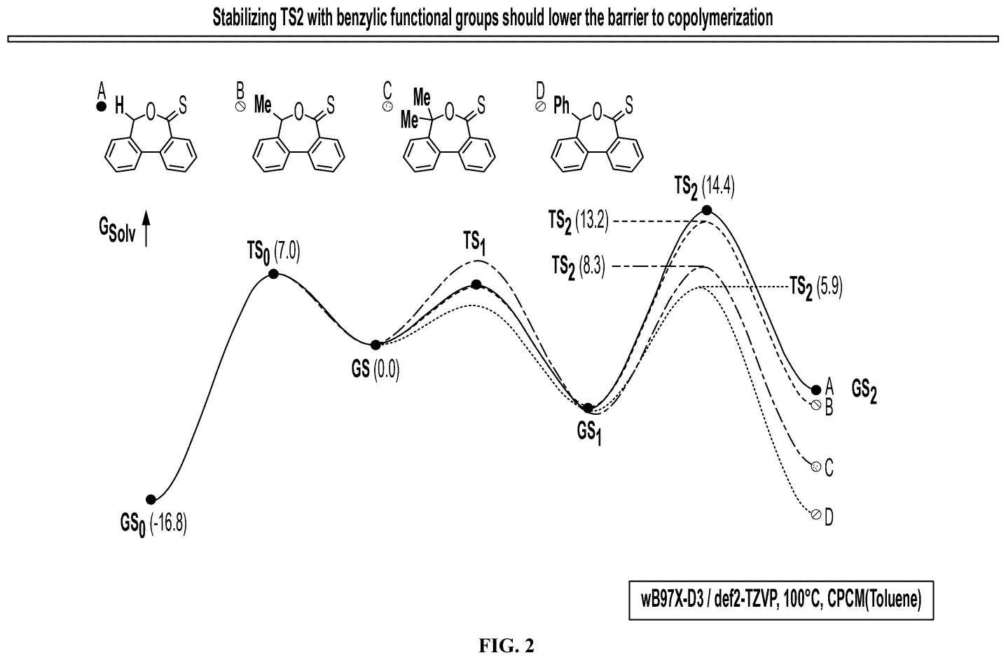

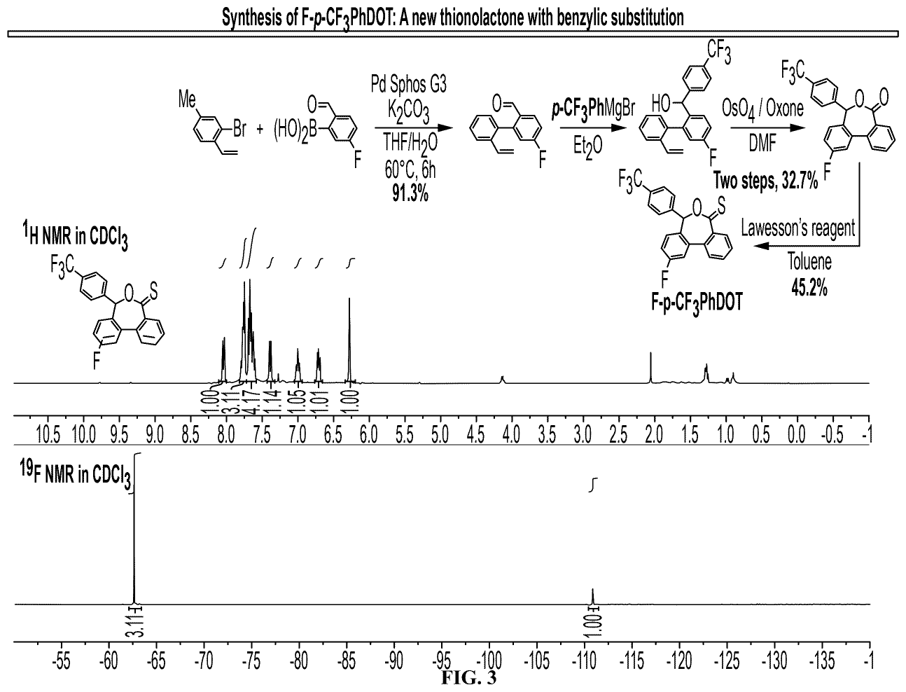

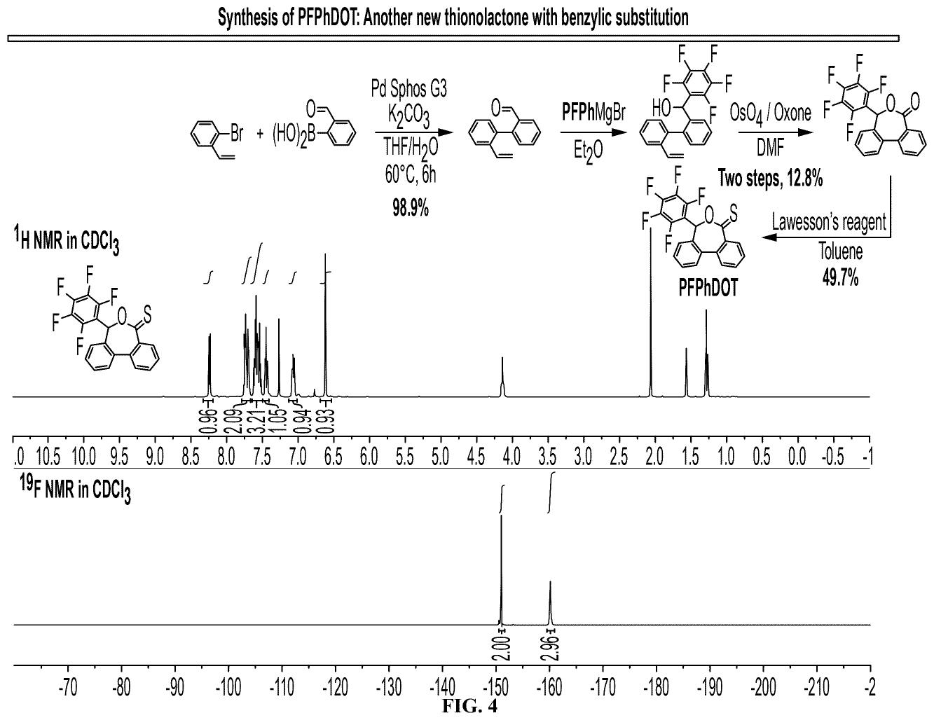

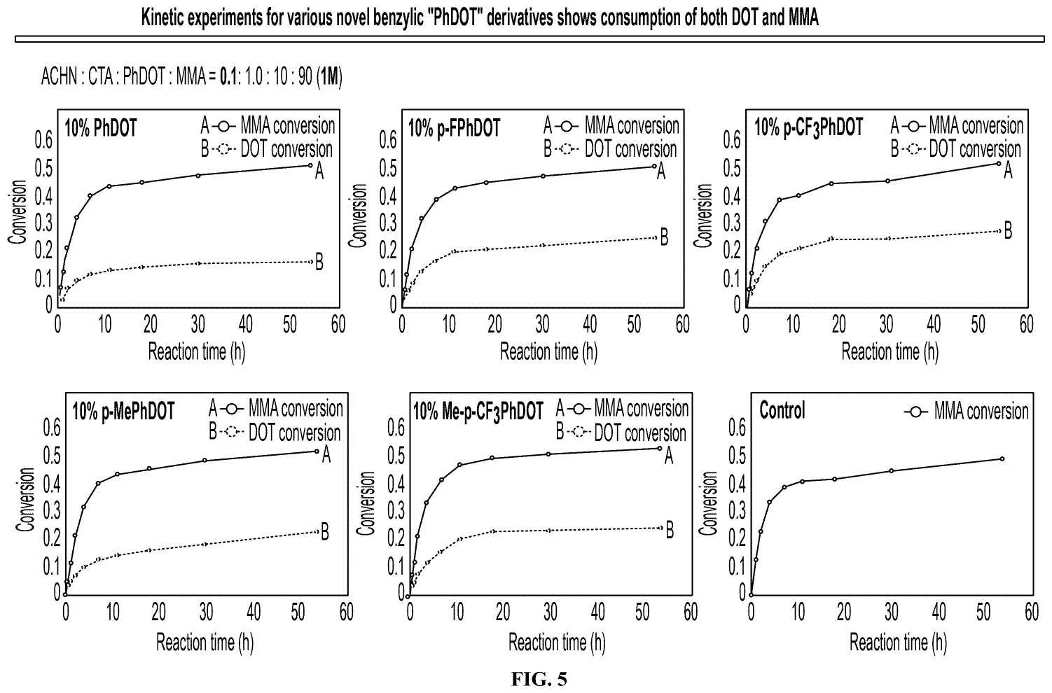

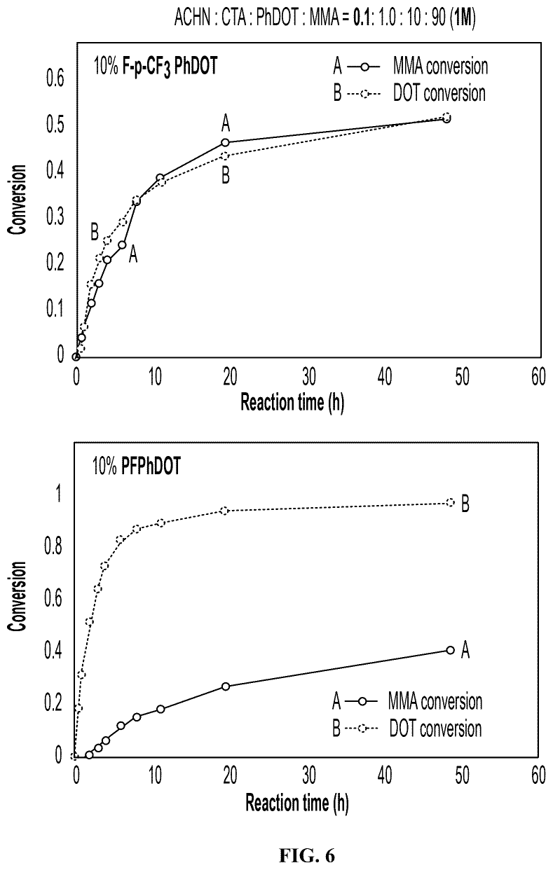

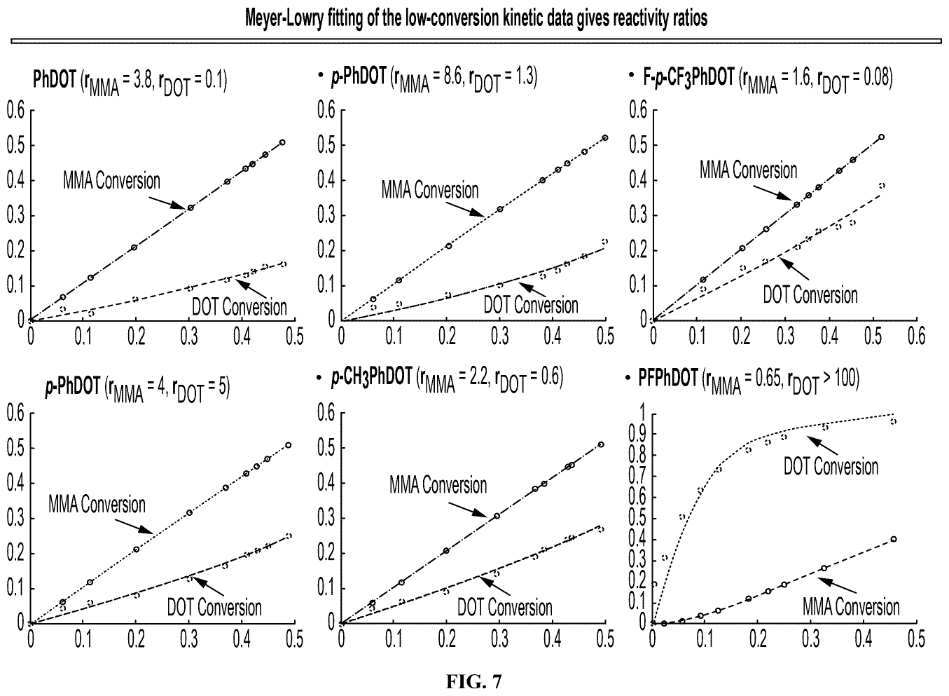

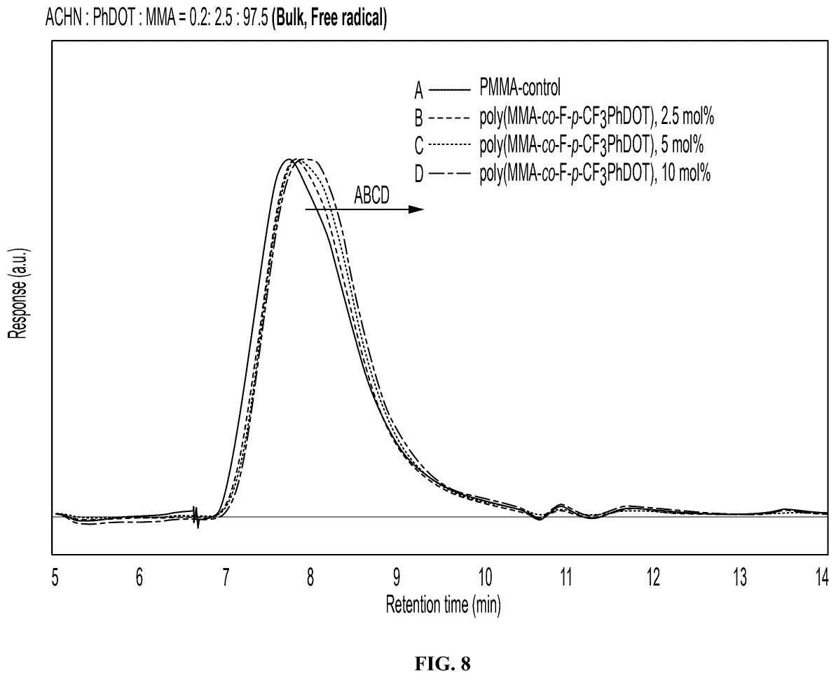

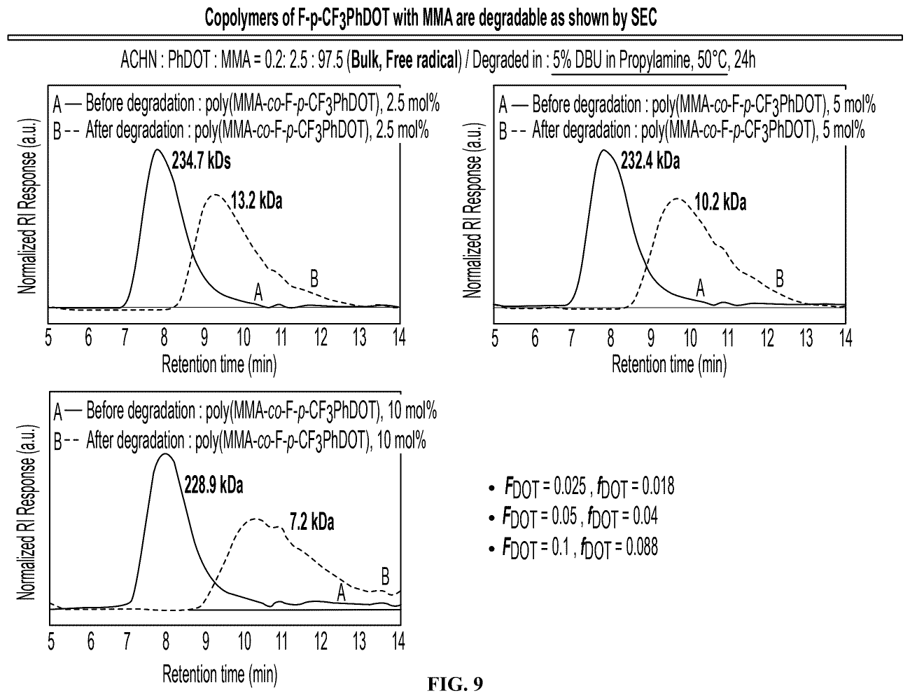

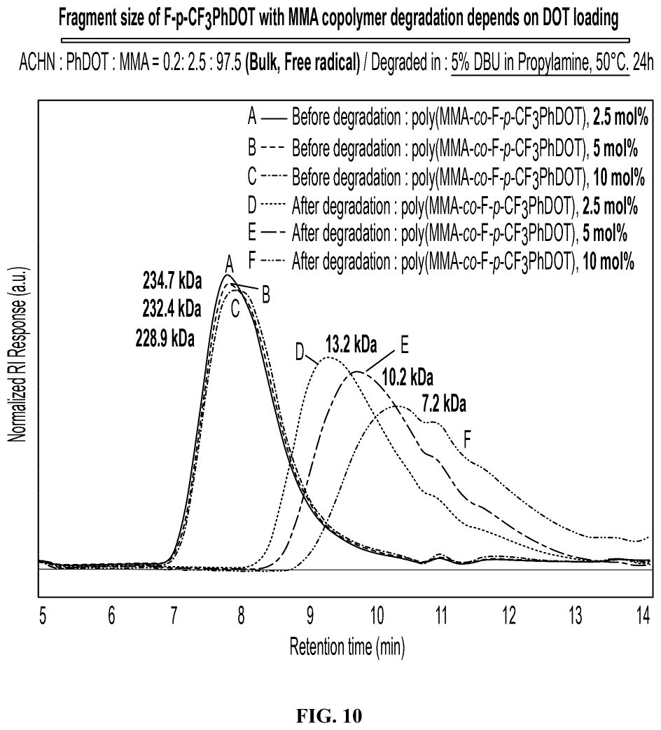

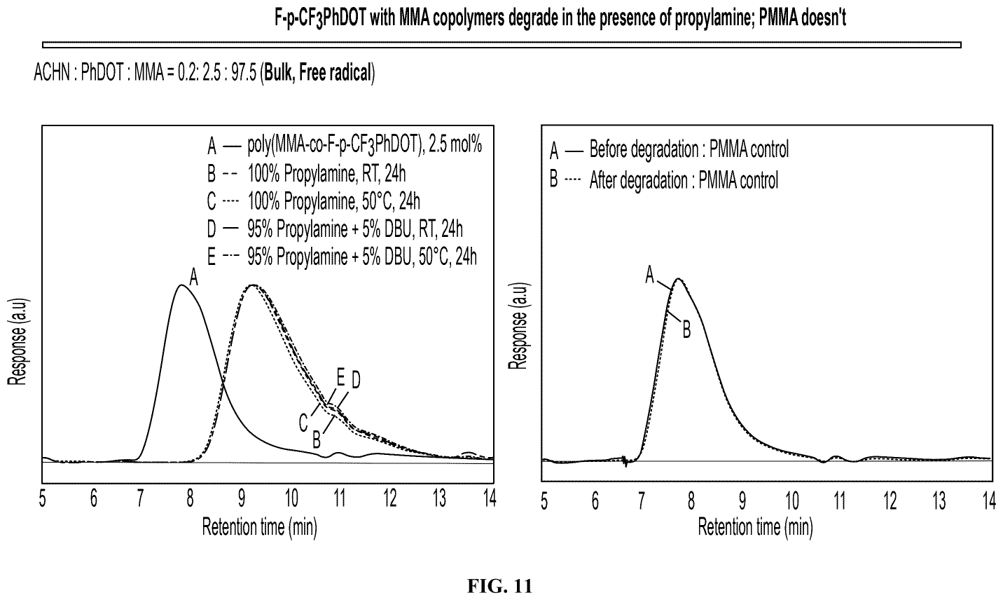

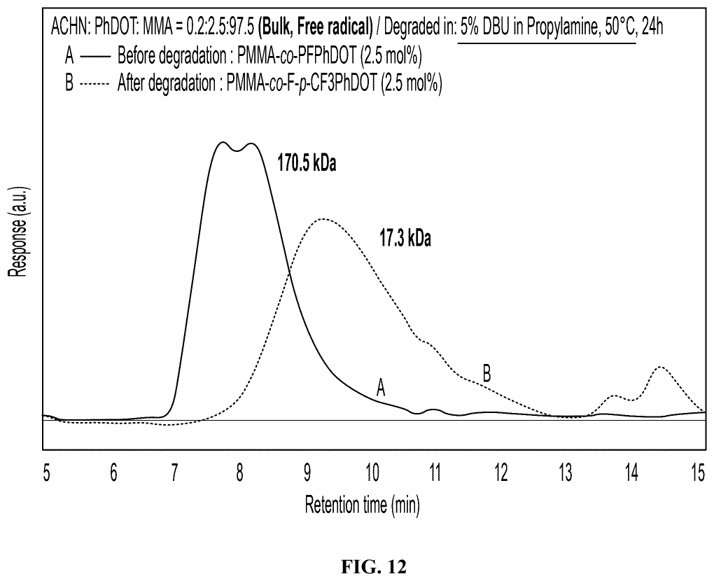

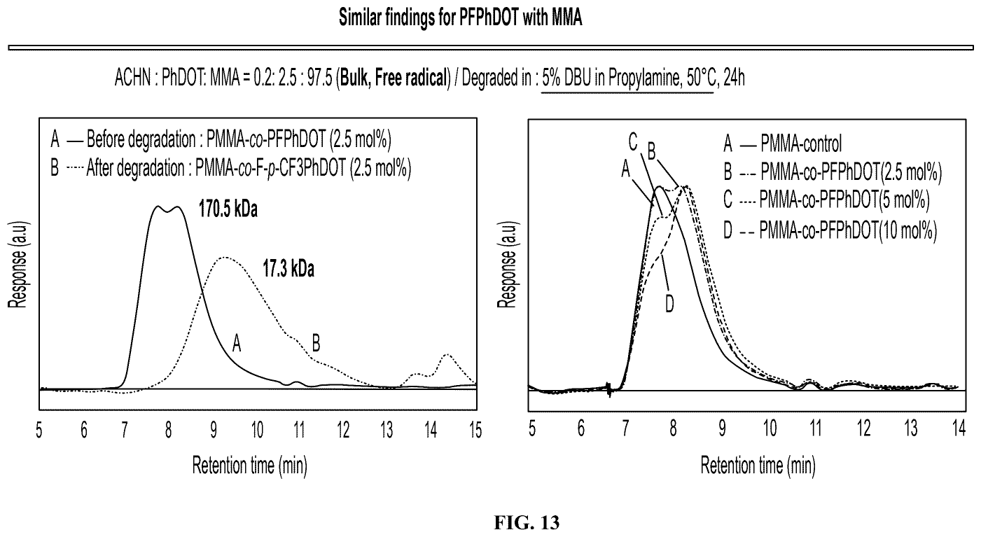

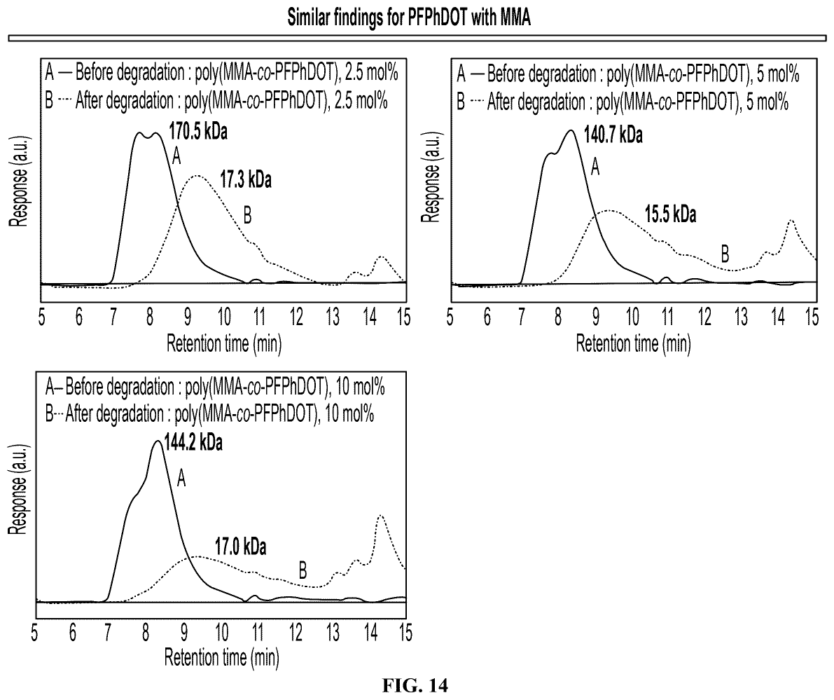

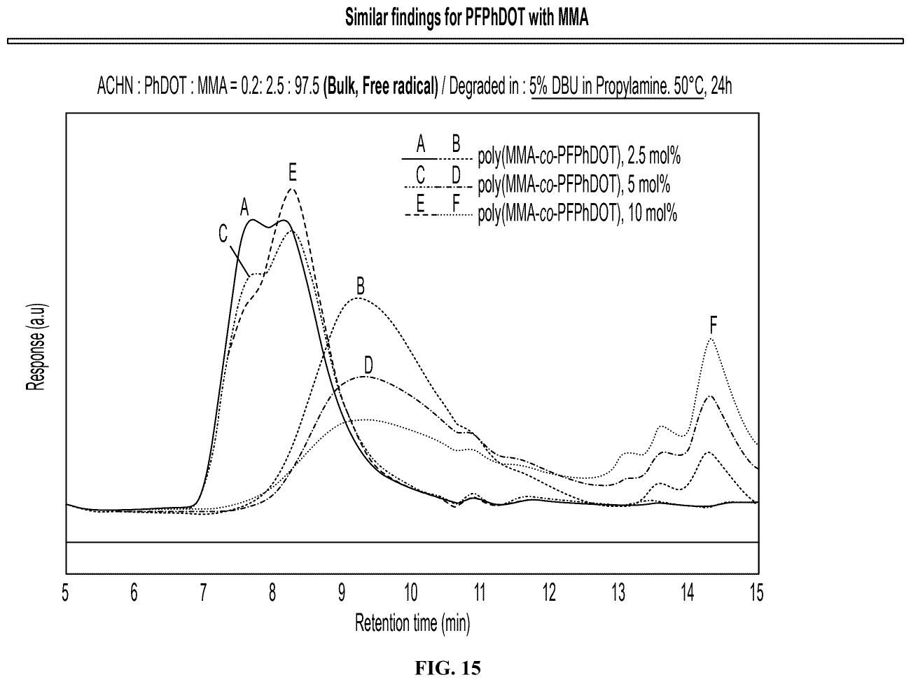

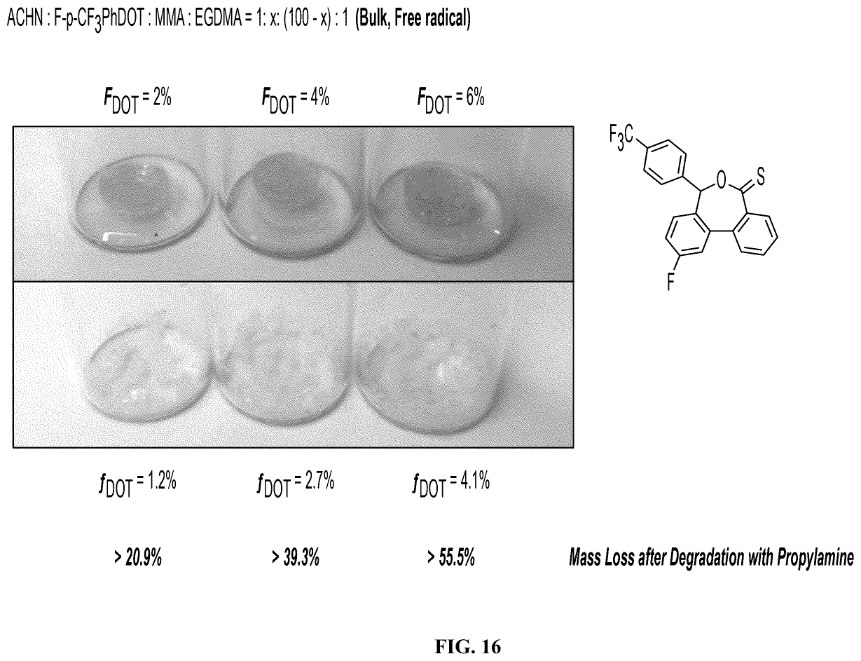

To test this hypothesis, the calculated relative Gibbs free energy profiles for 1 propagating with DOT were compared to bDOTs 7-methylDOT (MeDOT), 7,7-dimethylDOT (Me₂DOT), and 7-phenylDOT (PhDOT) (FIG. 2). All three derivatives were calculated to have a lower TS-D2 than the parent DOT. PhDOT was predicted to exhibit TS-D2 energy of 5.9 kcal/mol, which is ˜1.1 kcal/mol below that of TS-M1. Bolstered by these computational results, the synthesis of bDOTs was performed to experimentally assess their copolymerization reactivity with MMA.

Synthesis of bDOTs and Evaluation of Copolymerization Reactivity.

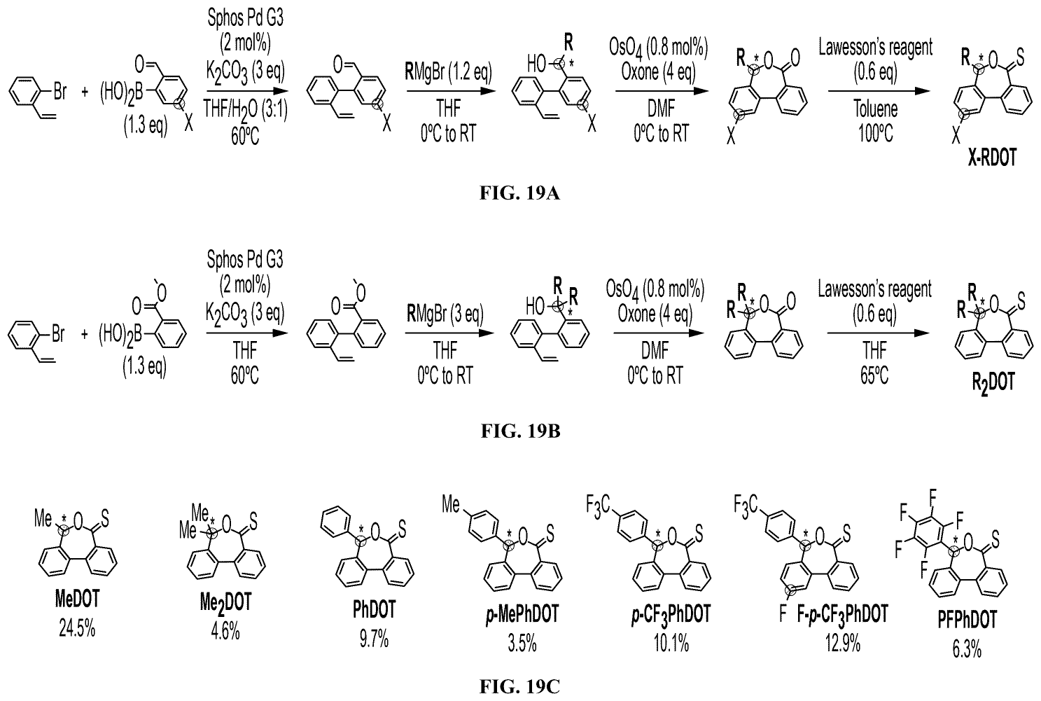

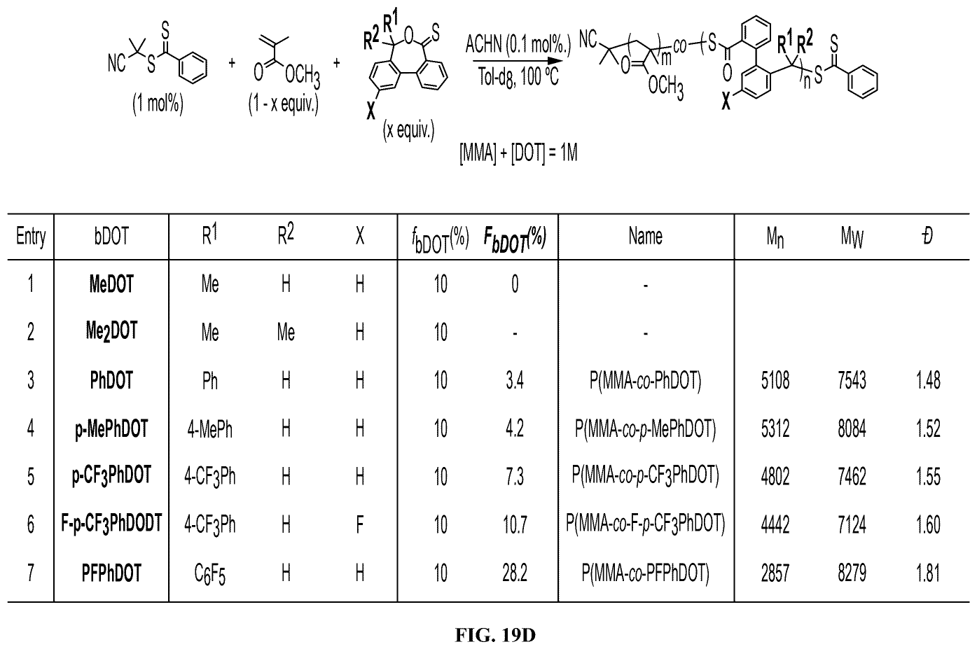

Synthetic routes were developed to facilitate the syntheses of bDOTs (FIGS. 19A-19B). A palladium-catalyzed Suzuki coupling involving 2-bromostyrene and the derivatives of either 2-formyl or 2-methoxycarbonyl phenylboronic acid was utilized, followed by a Grignard reaction that installed the benzylic substituents (circles marked by asterisks, FIGS. 19A-19C). The use of 2-formyl phenylboronic acid as a starting material allows for the introduction of one benzylic substituent, whereas 2-methoxycarbonyl phenylboronic acid enables two. Employing 4-substituted 2-formyl phenylboronic acid derivatives enables aryl ring functionalization (circles not marked by asterisks, FIGS. 19A-19C), potentially altering solubility and reactivity.⁸⁷ Subsequent to the introduction of benzylic substituent(s), oxidative lactonization followed by thionation furnishes the desired bDOT. The advantages of these pathways include: 1) the introduction of benzylic substitution(s) via a Grignard reaction, which allows for the incorporation of diverse functional groups depending on the chosen Grignard reagent, and 2) the capability to introduce either one or two benzylic substituents using an analogous route, an option not achieved with previous synthetic methods that relied on transesterification to form the lactone.^{78,79,87}

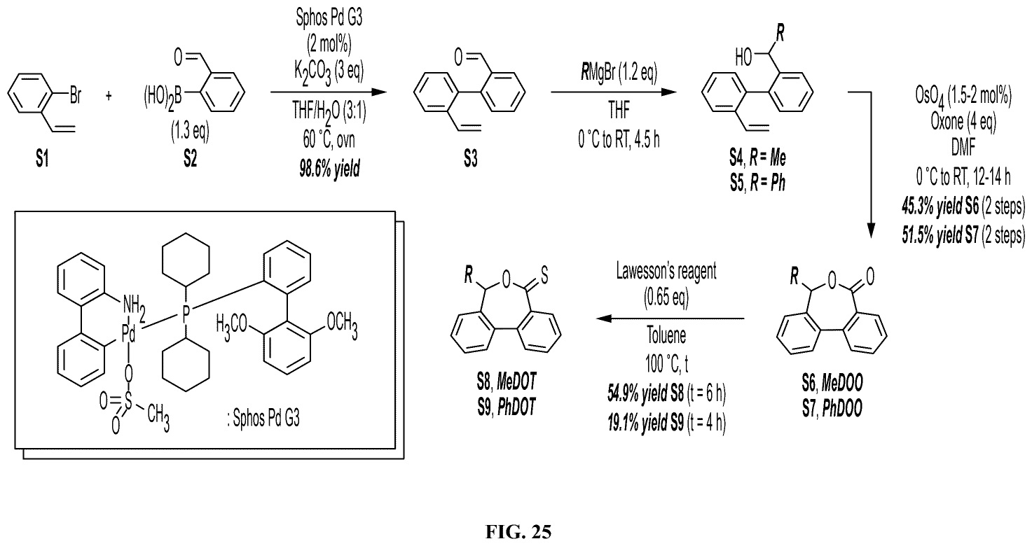

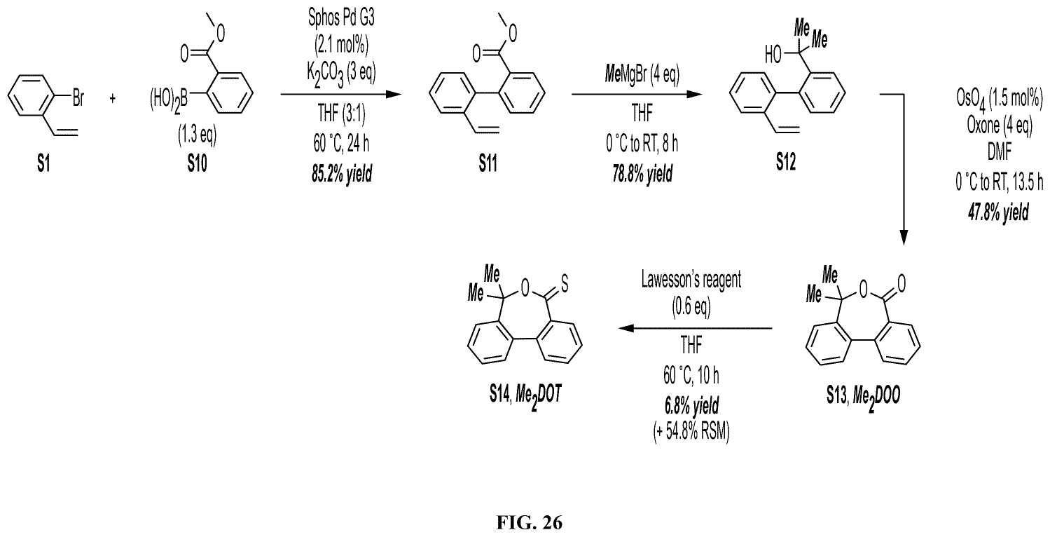

MeDOT and PhDOT were successfully synthesized from 2-bromostyrene and 2-formyl phenylboronic acid (FIG. 25). A Suzuki coupling employing the 3rd-generation Buchwald precatalyst Sphos Pd G3 yielded the coupled product 2′-vinyl-[1,1′-biphenyl]-2-carbaldehyde with a 98.6% yield. This step was followed by a Grignard reaction using MeMgBr and PhMgBr for MeDOT and PhDOT, respectively. Subsequent OsO₄-catalyzed oxidative cyclization formed lactones 7-methyldibenzo[c,e]oxepin-5(7H)-one (MeDOO) and 7-phenyldibenzo[c,e]oxepin-5(7H)-one (PhDOO), with two-step yields of 45.3% and 51.5%, respectively. The final step involved thionation using Lawesson's reagent to produce MeDOT and PhDOT. The overall yield for MeDOT synthesis (24.5%) was on par with that reported for DOT (26.6%), 79 with a thionation yield of 54.9%. By contrast, PhDOT was synthesized at a lower overall yield of 9.7% due to its reduced thionation yield of 19.1%. The major side products of the thionation were identified as the isomerized thiolactone 7-phenyldibenzo[c,e]thiepin-5(7H)-one (PhDTO) and the overthionated 7-phenyldibenzo[c,e]thiepine-5(7H)-thione (PhDTT). Me₂DOT was synthesized beginning with an Sphos Pd G3-catalyzed Suzuki coupling between 2-formyl phenylboronic acid and 2-methoxycarbonyl phenylboronic acid, achieving a coupling yield of 85.2%. Subsequently, a Grignard reaction introduced two methyl groups with a 78.8% yield, and OsO₄-mediated lactonization yielded 7,7-dimethyldibenzo[c,e]oxepin-5(7H)-one (Me₂DOO) with a 47.8% yield. For the thionation step, a reduced temperature (65° C.) and an alternative solvent (THF) were employed to mitigate excessive isomerization. The thionation yield was 5.8% (FIG. 26).

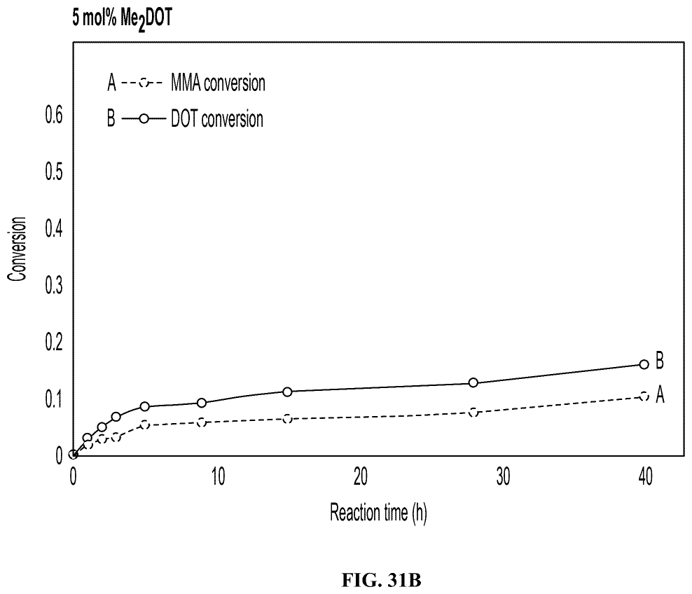

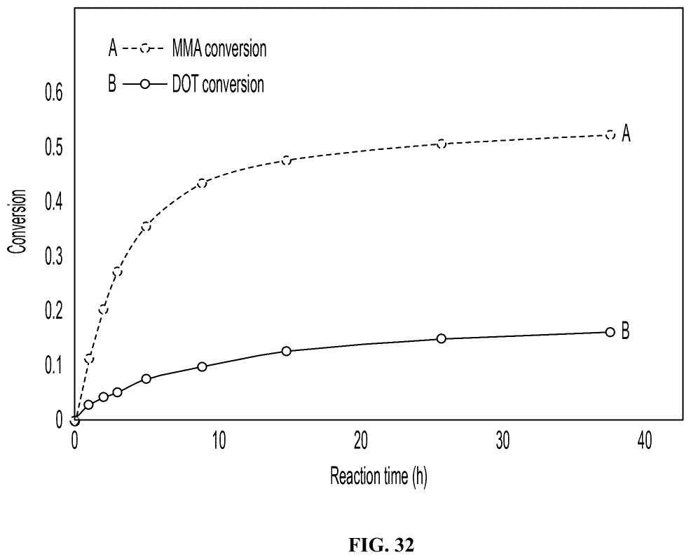

Each bDOT (initial feed f_{bDOT}=10 mol %) was tested for its potential to undergo copolymerization with MMA under reversible addition-fragmentation chain transfer (RAFT) polymerization conditions, with 1,1′-azobis(cyanocyclohexane) (ACHN) as the initiator, 1 mol % 2-cyano-2-propyl benzodithioate as the chain transfer agent (CTA), and a reaction temperature of 100° C. (FIG. 19D). RAFT conditions facilitated the measurement of copolymerization kinetics and the determination of reactivity ratios by suppressing competing termination pathways in free radical copolymerization. bDOT conversions were measured as a function of time through quantitative ¹H NMR. As with DOT, no conversion was observed for MeDOT (FIG. 31A). While Me₂DOT displayed a gradual transformation into an unidentified species, it also did not participate in copolymerization with MMA (FIG. 31B). PhDOT was consumed throughout its copolymerization with MMA to produce copolymer P(MMA-co-PhDOT) (FIG. 32). No isomerization from TL to the corresponding thiolactone was observed for any of the bDOTs under these copolymerization conditions.

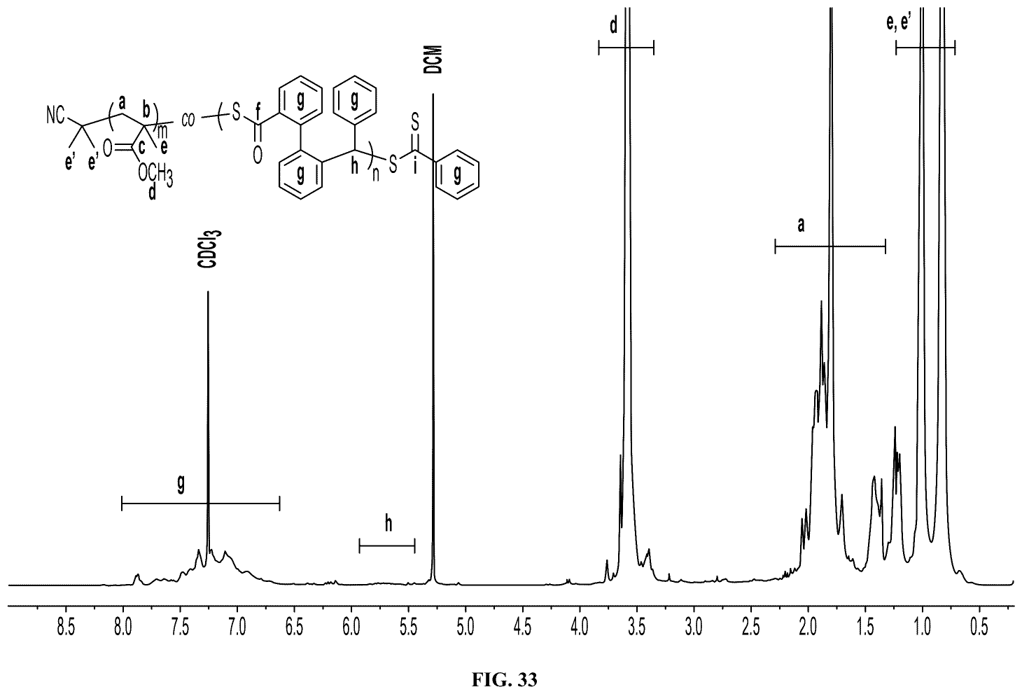

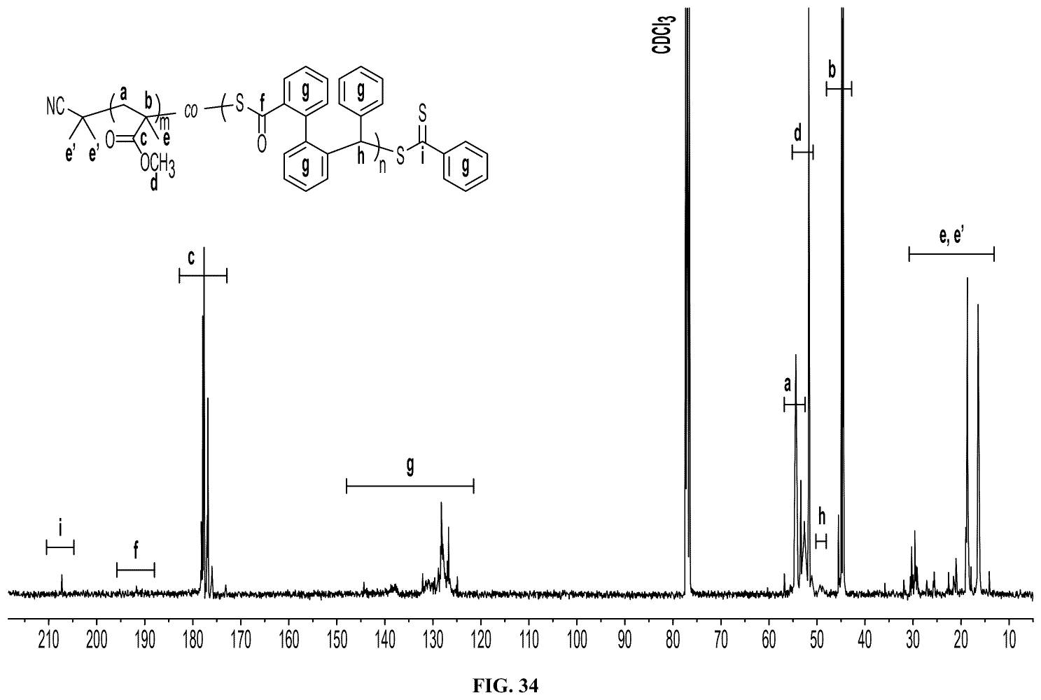

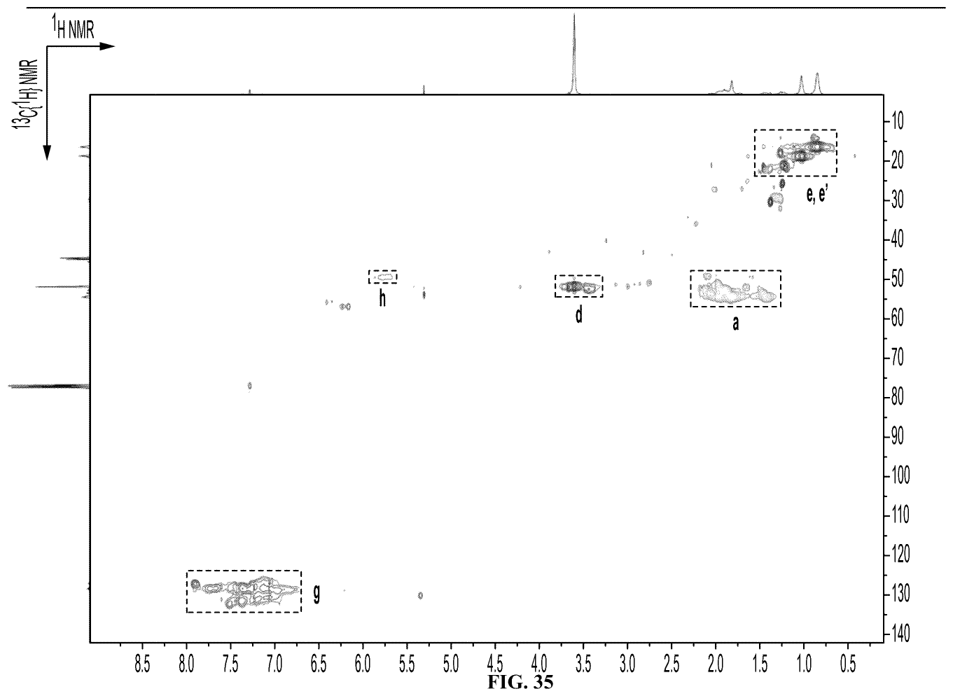

¹³C NMR analysis of the purified P(MMA-co-PhDOT) showed signals at δ=191 ppm, characteristic of thioester carbons, which is consistent with the successful incorporation of PhDOT into the polymer (FIG. 34). Moreover, the complete disappearance of the singlet benzylic C—H peak at δ=6.34 ppm and its shift to δ=5.5-5.9 ppm suggested that every introduced comonomer underwent ring-opening to contribute a thioester to the polymer (FIG. 33). The overall incorporation (F_{bDOT}) of PhDOT, however, was modest as determined by quantitative ¹H NMR analysis, where F_{bDOT} was only 3.4% for an f_{bDOT} of 10%.

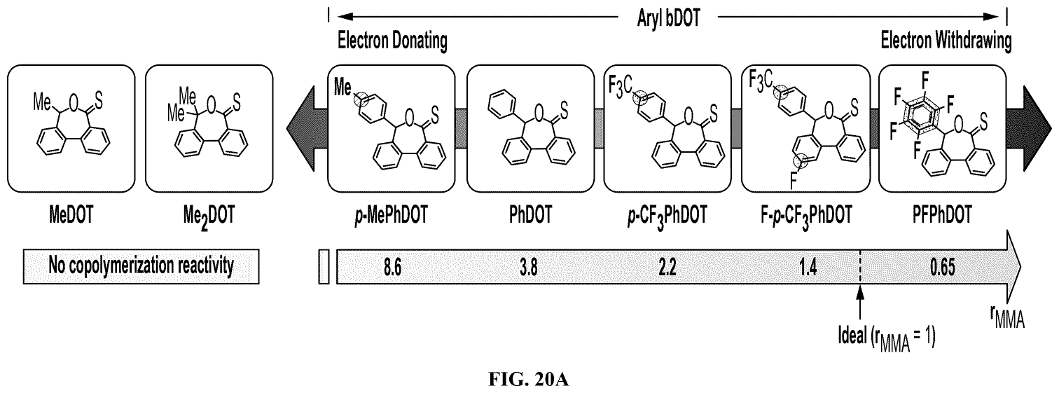

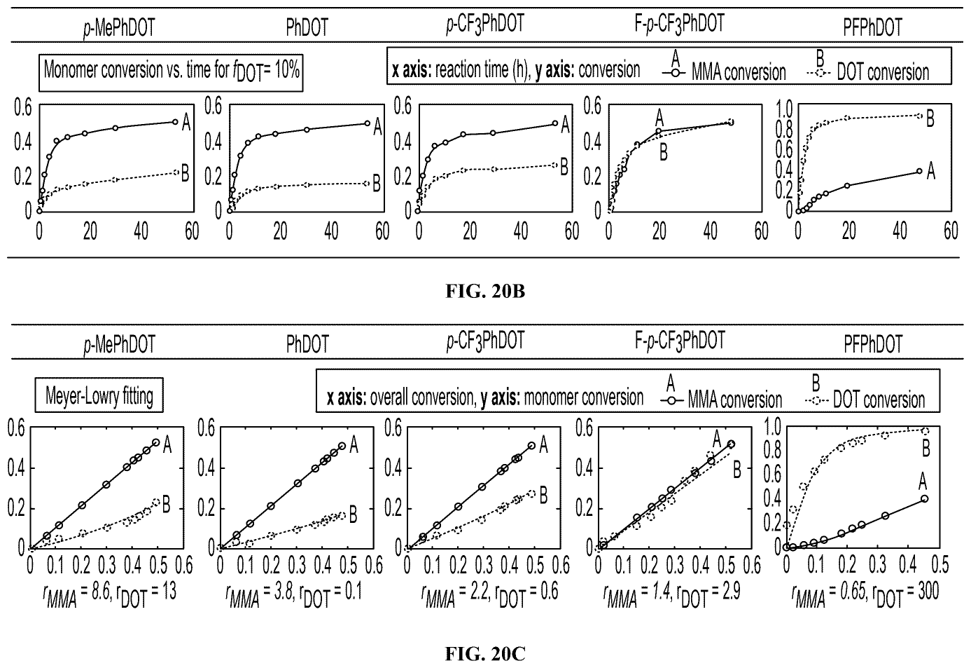

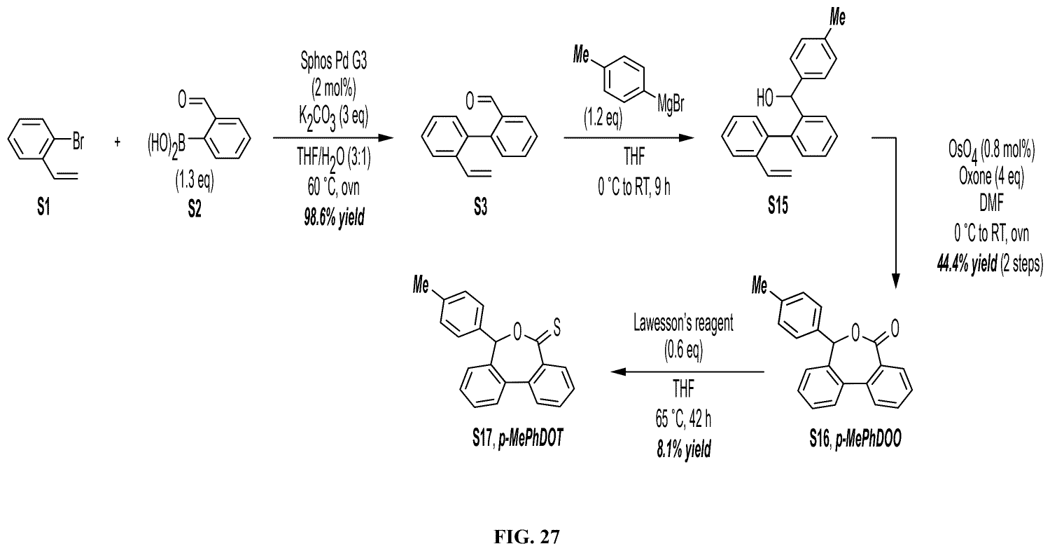

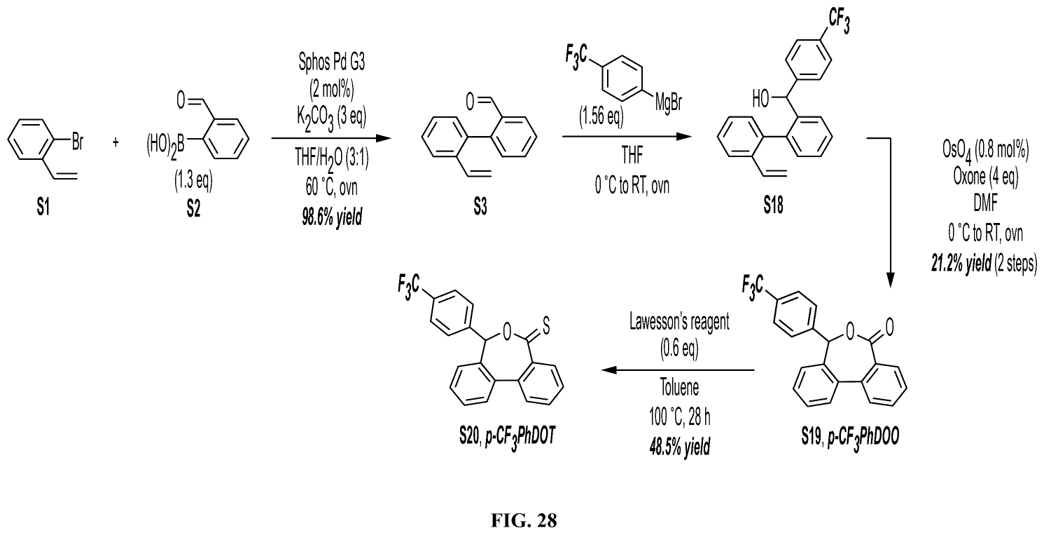

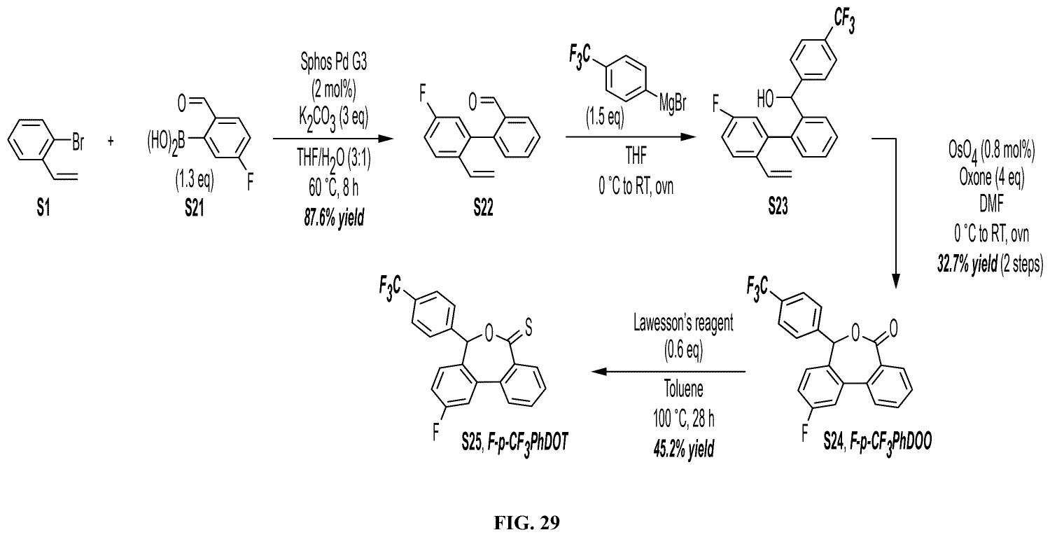

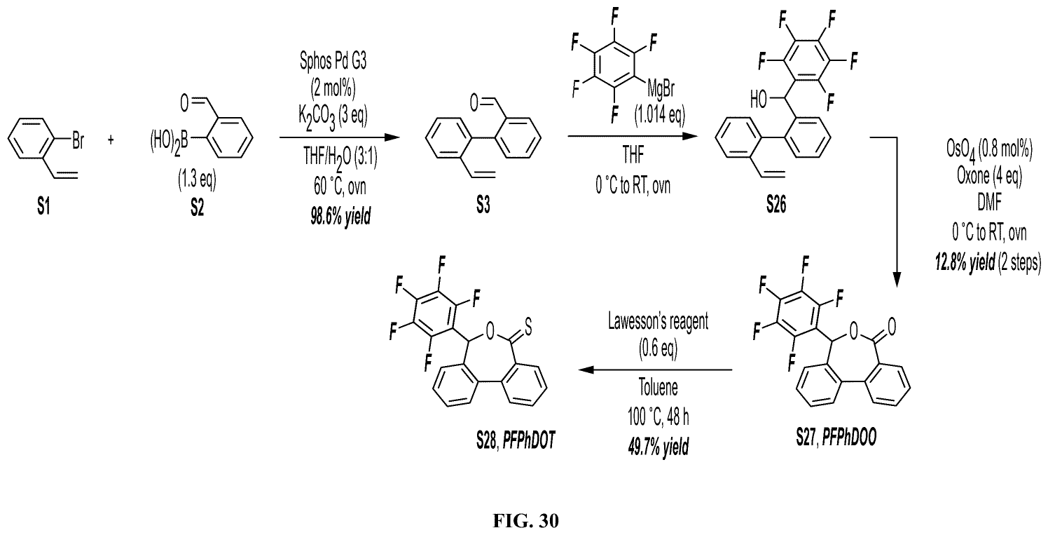

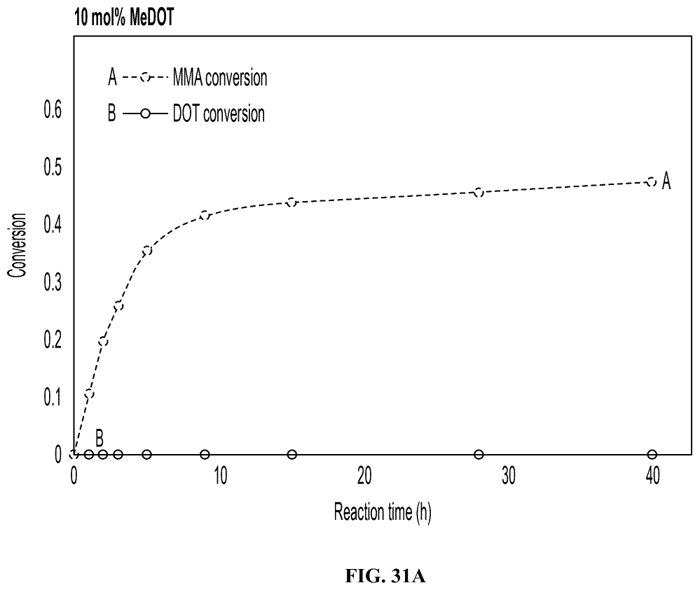

Enhanced bDOT reactivity was pursued by further modulating the electronic properties of the benzylic substituent for impacting the stability of the transition state radical (vide supra). Para Me and CF₃ substituents were introduced onto the phenyl ring of PhDOT using the appropriate phenyl Grignard reagent, generating bDOTs p-MePhDOT (FIG. 27) and p-CF₃PhDOT (FIG. 28), respectively. Aryl bDOTs with stronger electron-withdrawing character, 10-fluoro-7-(4-(trifluoromethyl)phenyl)DOT (F-p-CF₃PhDOT) and 7-(pentafluorophenyl)DOT (PFPhDOT), were also prepared (FIG. 20A). F-p-CF₃PhDOT was synthesized starting from 5-fluoro-2-formylphenylboronic acid following an otherwise identical synthetic route to p-CF₃PhDOT (FIG. 29). PFPhDOT was produced using the pentafluorophenyl Grignard reagent (FIG. 30). A correlation between the electron withdrawing ability of the benzylic substituent and the stability of the resulting bDOT toward isomerization to the corresponding thiolactone (and thus the synthesis yield) under thionation conditions was observed. For example, the thionation yields of p-CF₃PhDOT, F-p-CF₃PhDOT, and PFPhDOT were 48.5%, 45.2%, and 49.7%, respectively, which were much higher than that for PhDOT (19.1%). By contrast, the synthesis of p-MePhDOT involved a combination of reduced temperature (65° C.) and THF as a solvent to avoid excessive isomerization, leading to a thionation yield of 8.1% (FIG. 27). Overall, these findings are consistent with the build-up of a partial positive charge on the benzylic carbon during the transition state of the isomerization reaction.

Conversions of MMA and each aryl bDOT (p-MePhDOT, PhDOT, p-CF₃PhDOT, F-p-CF₃PhDOT, and PFPhDOT; f_{bDOT}=10 mol %) were measured versus time via ¹H NMR using the RAFT copolymerization conditions outlined above (FIG. 20B). The conversion data were fitted to the integrated terminal copolymerization model reported by Meyer-Lowry,^{91,92} providing r_{MMA} values of 8.6 for p-MePhDOT, 3.8 for PhDOT, 2.2 for p-CF₃PhDOT, 1.4 for F-p-CF₃PhDOT, and 0.65 for PFPhDOT (FIG. 20C). These findings suggest that electron withdrawing groups may increase the reactivity of these bDOTs, shifting the r_{MMA} to smaller values; r_{MMA} approaches the ideal value of 1 as it transitions from p-MePhDOT to F-p-CF₃PhDOT and falls below 1 for PFPhDOT. The r_{DOT} values for p-MePhDOT, PhDOT, p-CF₃PhDOT, and F-p-CF₃PhDOT were measured to be within the range of 0.1 to 13; the variation of r_{DOT} in this range may not significantly affect the deconstruction efficiency, assuming an f_{bDOT} of 2.5% (FIG. 17A); however, for PFPhDOT, r_{DOT} was measured to be larger than 100.

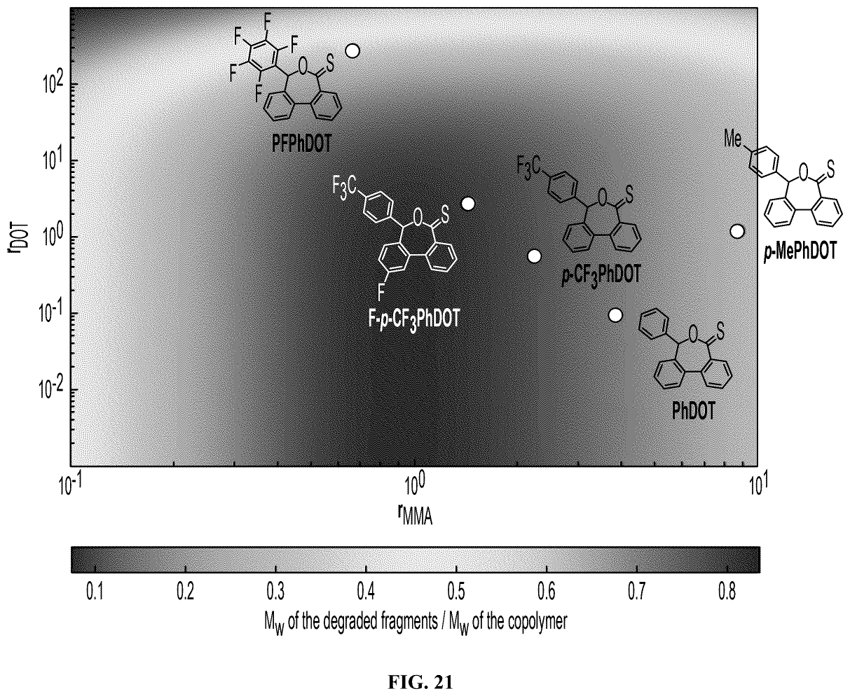

The efficiency of the aryl bDOTs as CCs can be predicted by comparing them on the heat map from Monte-Carlo simulations based on their experimentally measured reactivity ratios (FIG. 21). p-MePhDOT, with a large r_{MMA} of 8.6 and an r_{DOT}>1, may form a blocky copolymer with MMA, with the MMA blocks predominantly appearing earlier than the p-MePhDOT blocks, rendering it less efficient as a CC. PhDOT and p-CF₃PhDOT, with their r_{MMA}>1 and r_{DOT}<1, may form gradient copolymers with MMA, with earlier segments of these copolymers comprising mostly MMA and the CCs distributed in the latter segments. Among these two, p-CF₃PhDOT may produce relatively less gradient in the polymer and accordingly, smaller deconstruction fragment sizes due to its smaller r_{MMA}. F-p-CF₃PhDOT, with an r_{MMA} of 1.4 and an r_{DOT} of 2.9, may produce copolymers with slight blockiness; however, its r_{MMA} close to 1 may result in a well-distributed CC composition in the copolymer chains, making it highly efficient as a CC.F-p-CF₃PhDOT is positioned on the heatmap (FIG. 21) in a region suggesting nearly ideal deconstruction into small molar mass fragments. PFPhDOT, despite its r_{MMA} being close to 1, has an exceedingly large r_{DOT}, indicating a strong propensity for homopropagation. Therefore, its copolymer with MMA may have PFPhDOT blocks at the earlier parts of the chains with negligible distribution in the latter parts, making it less efficient as a CC. Overall, these results demonstrate a significant improvement in understanding and tuning bDOT CC performance, facilitated by mechanism-guided molecular design.

Application to F-p-CF₃PhDOT PMMA synthesis via free radical copolymerization.

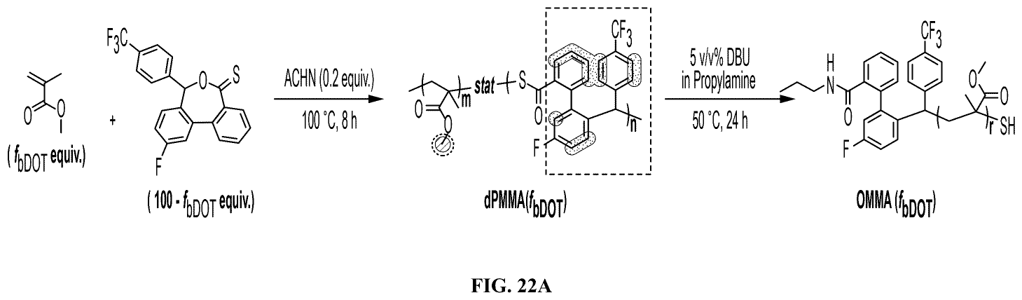

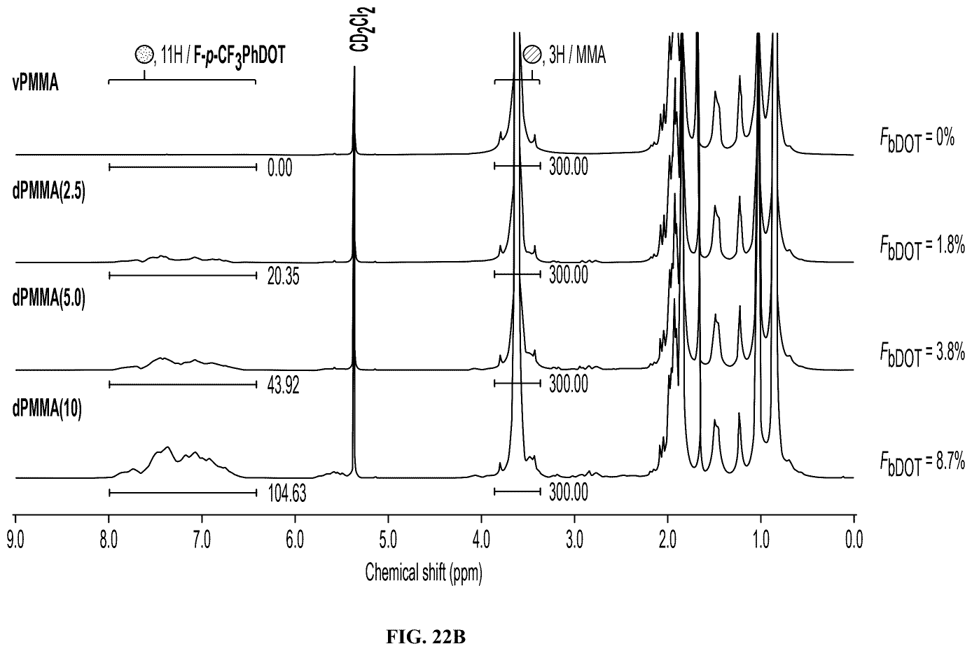

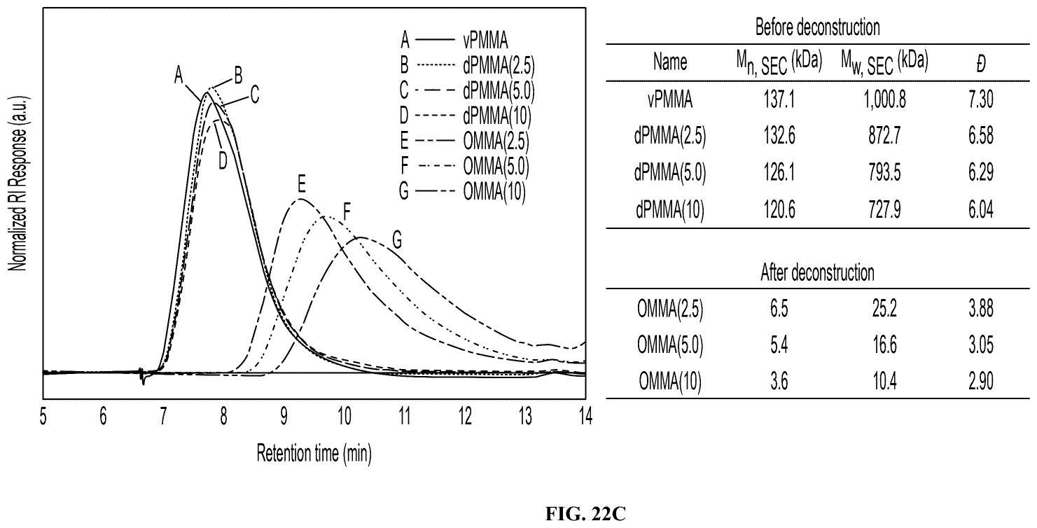

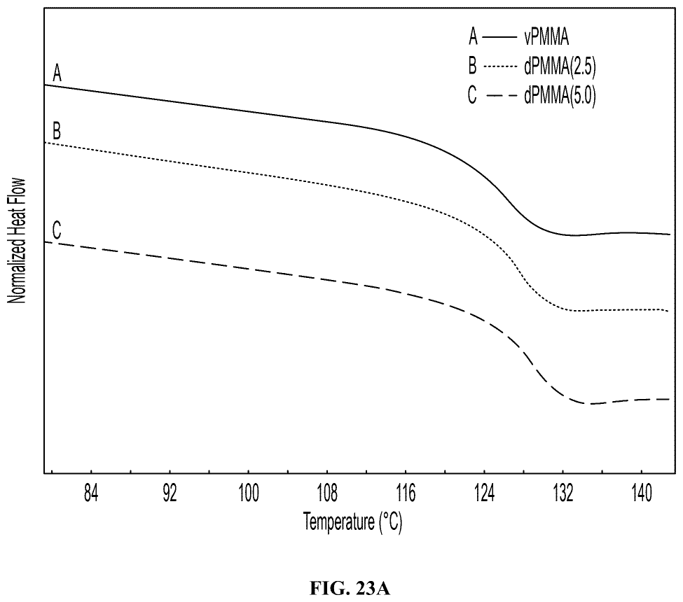

Having identified F-p-CF₃PhDOT as a promising candidate for copolymerization with MMA, its performance was subsequently evaluated under solvent-free FRP conditions used for high molecular weight PMMA synthesis in industry⁸⁹ (Note: F-p-CF₃PhDOT displayed good solubility in MMA: >233 mg/mL). F-p-CF₃PhDOT was combined with MMA in varied feed ratios (f_{bDOT}=0% (control), 2.5%, 5%, and 10%) and heated to 100° C. in the presence of ACHN for 8 hours (FIG. 22A). The resulting virgin PMMA (f_{bDOT}=0%, vPMMA) and the copolymers, dPMMA(f_{bDOT}), were isolated by repeated precipitation in methanol. Quantitative ¹H NMR analysis of the isolated copolymers showed F_{bDOT} values of 1.8%, 3.8%, and 8.7% for the f_{bDOT}=2.5%, 5.0%, and 10%, respectively (FIG. 22B). The number-average molar masses (M_{n,SEC}) of vPMMA, dPMMA(2.5), dPMMA(5.0), and dPMMA(10) were all above 120 kDa as determined by size exclusion chromatography (SEC, FIG. 22C).

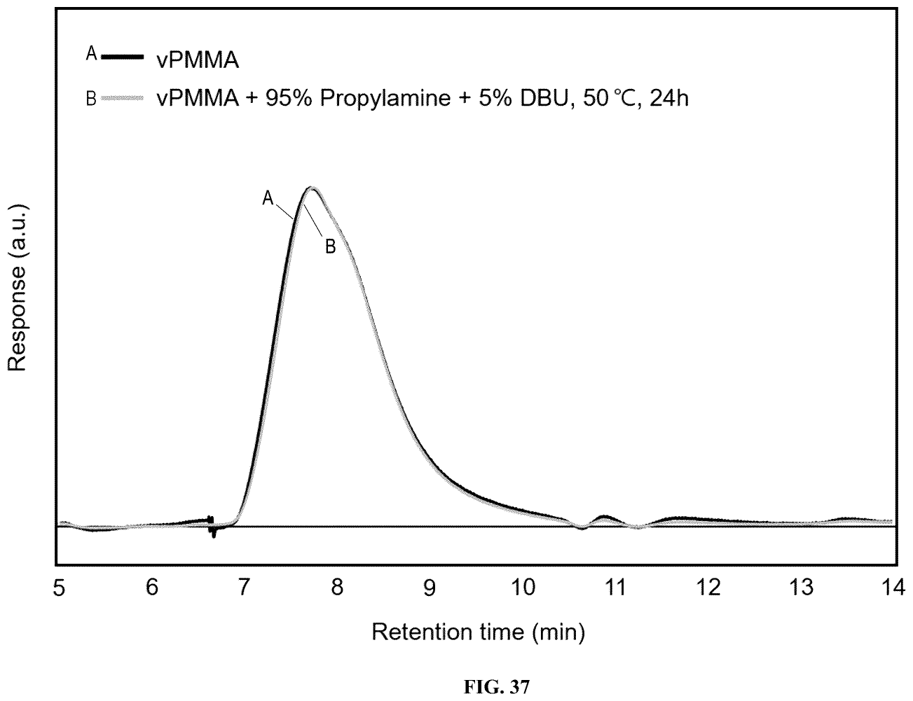

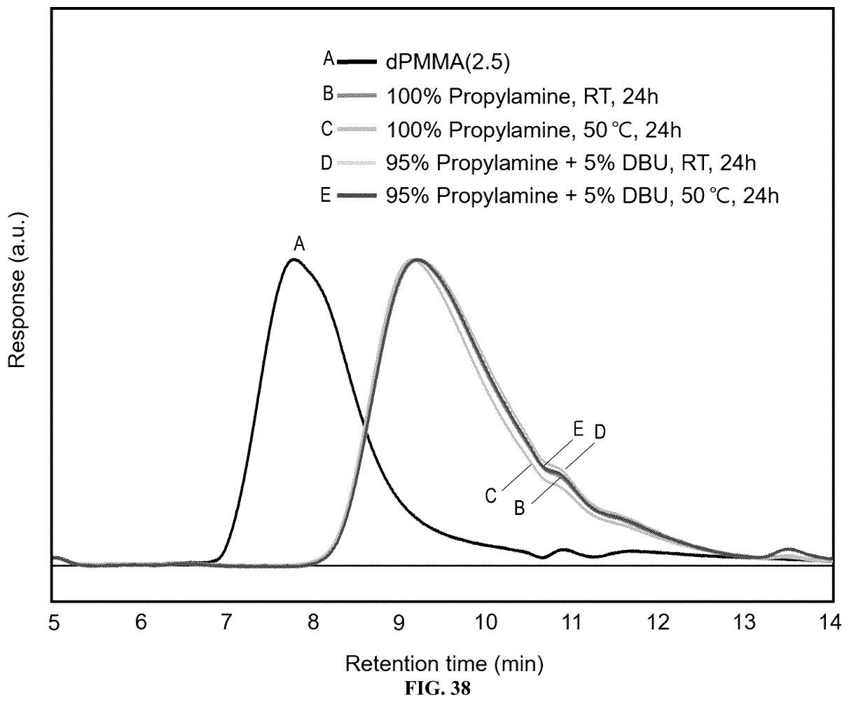

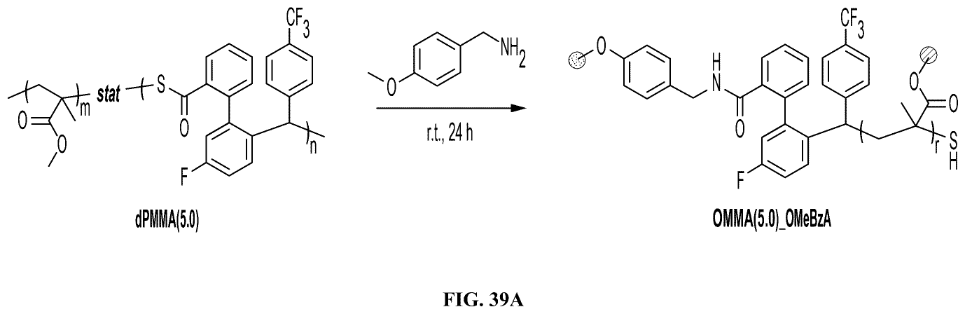

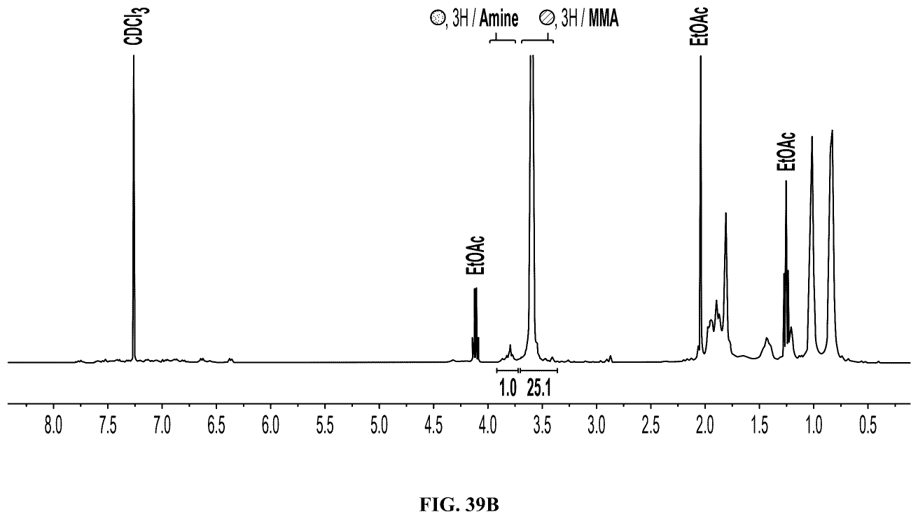

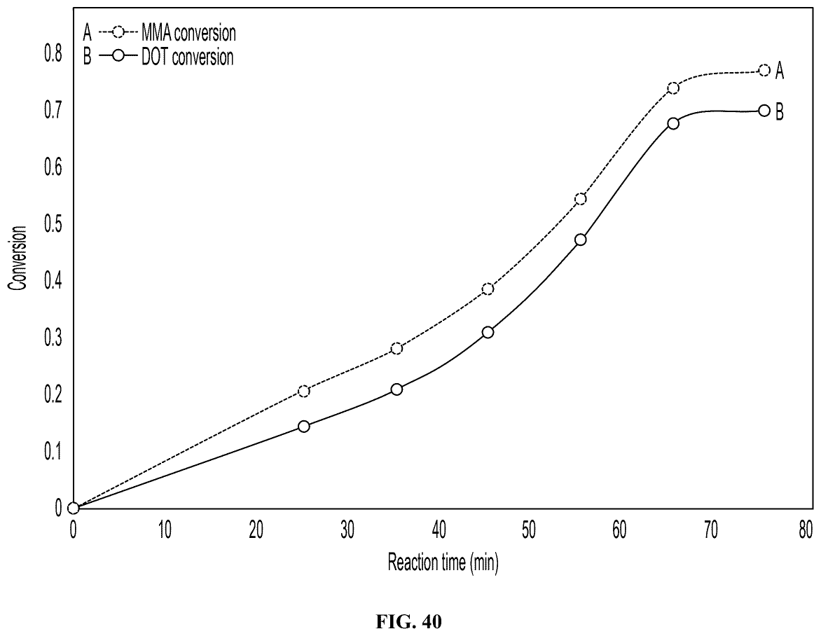

Deconstruction of copolymers dPMMA(2.5), dPMMA(5.0), and dPMMA(10) was achieved by treating each sample with 5 v/v % DBU in neat propylamine at 50° C. for 24 hours (FIG. 22A). Exposing vPMMA to these conditions did not lead to any observable decrease in molar mass (FIG. 37). By contrast, the bDOT-containing copolymers underwent deconstruction into low molar mass oligomethylmethacrylate (OMMA) fragments designated as OMMA(f_{bDOT}). M_{n,SEC} decreased by more than 20-fold with bDOT loading, with OMMA(2.5), OMMA(5.0), and OMMA(10) having an M_{n,SEC} of 6.5 kDa, 5.3 kDa, and 3.6 kDa, respectively (FIG. 22C). Similarly, M_{w,SEC}, for OMMA(2.5), OMMA(5.0), and OMMA(10) decreased by more than 20-fold to 25.2 kDa, 16.6 kDa, and 10.4 kDa, respectively. Complete deconstruction was also achievable under milder conditions, as demonstrated for dPMMA(2.5), using neat propylamine at room temperature (FIG. 38). Altogether, these results demonstrate that F-p-CF₃PhDOT efficiently copolymerizes with MMA under free radical polymerization conditions to generate high molecular weight, deconstructable copolymers that can be cleaved into oligomers with >20-fold reduction in molar mass. These results represent an example of a thionolactone comonomer that undergoes efficient copolymerization with MMA.

Properties of deconstructable PMMA.

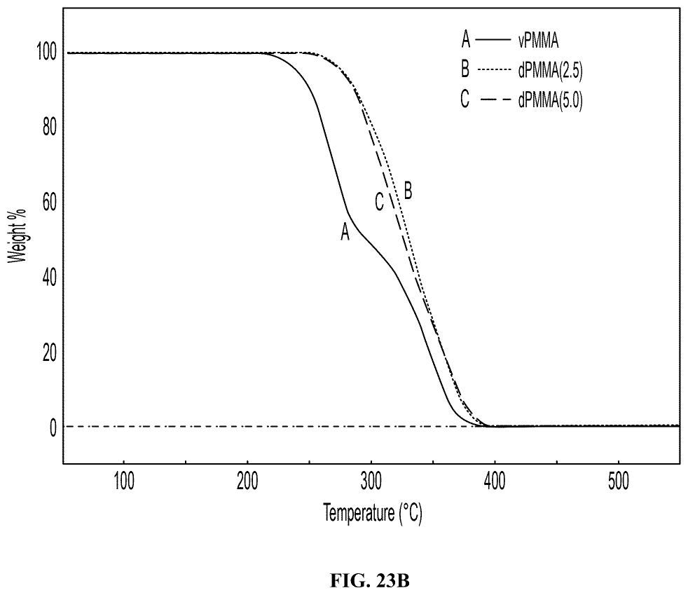

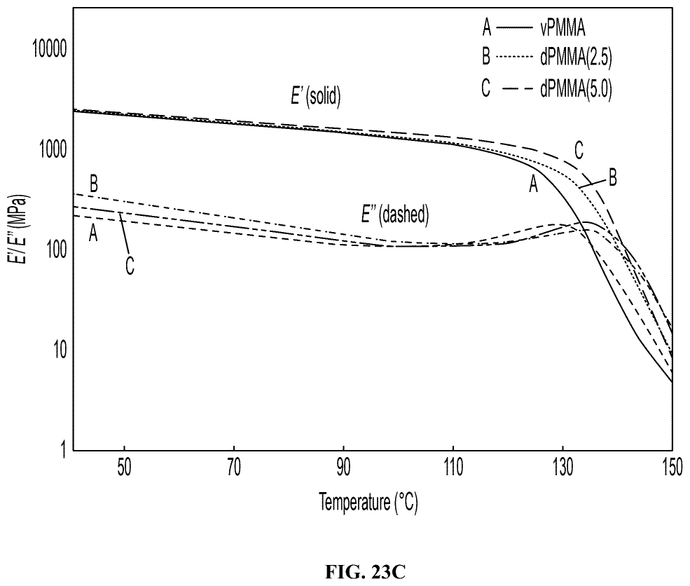

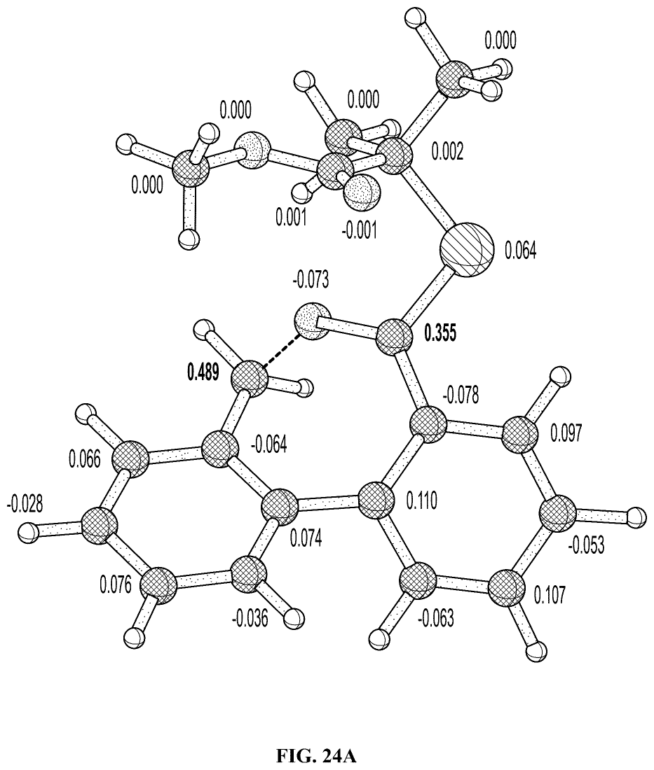

As noted above, an ideal CC should not negatively impact the desirable properties of the polymer in which it is used. One of the properties of PMMA is its colorlessness and transparency after processing, which allows for its application as glass substitutes. Although F-p-CF₃PhDOT is yellow, it loses its color once polymerized, resulting in vPMMA, dPMMA(2.5), and dPMMA(5.0) being colorless and visually indistinguishable. Purified vPMMA, dPMMA(2.5), and dPMMA(5.0) showed no difference in color. The transparency of vPMMA and dPMMA(5.0) after compression molding also showed no notable difference. The thermal properties of dPMMA(2.5) and dPMMA(5.0) were investigated and compared to that of vPMMA using differential scanning calorimetry (DSC) and thermal gravimetric analysis (TGA). The glass transition temperatures (T_{g}) of dPMMA(2.5) and dPMMA(5.0) were 133.0° C. and 134.0° C., respectively, which are slightly higher yet nearly indistinguishable from the 131.9° C. measured for vPMMA (FIG. 23A). The thermal decomposition temperatures at 5% mass loss (Td, 5%) exhibited moderate increase, shifting from 239° C. to 275° C. for dPMMA(2.5) and to 274° C. for dPMMA(5.0) (FIG. 23B). The thermomechanical properties of dPMMA(2.5) and dPMMA(5.0) were further studied using dynamic mechanical analysis (DMA). Compression-molded rectangular bars displayed temperature sweep curves nearly identical to those of vPMMA, except for a slight increase in T_{g} that was observed with increased f_{bDOT}. The elastic moduli (E′) were consistent across vPMMA, dPMMA(2.5), and dPMMA(5.0), with values of 2.61±0.15 GPa, 2.79±0.33 GPa, and 2.87±0.21 GPa (averaged from 25-30° C. for triplicate runs), respectively, aligning well with the reported values for commercial PMMA (2.76-3.30 GPa⁹³) (FIG. 23C). These findings showed that while F-p-CF₃PhDOT provided thioester-based deconstructable PMMA, it did not negatively impact the properties of PMMA, and instead led to a slight increase in T_{g}.

In summary, this example describes an efficient TL CC for methacrylates and demonstrates its excellent compatibility with solvent-free FRP. This cleavable-comonomer may be useful for polymethacrylate end-of-life management, along with various orthogonal depolymerization strategies.⁹⁴⁻¹⁰⁷ Central to this advancement was the realization from mechanistic analysis that: 1) lowering the transition state energy of the DOT ring-opening step may be important to modulating r_{MMA} to approach the ideal value of 1; and 2) such lowering may be achieved through benzylic functionalization.

### General Experimental Details

### General Considerations

All reactions were performed using standard Schlenk techniques unless stated otherwise. All glassware was dried in a 120° C. oven overnight or flame-dried prior to use. Molecular sieves (4 Å) were activated by heating at 220° C. for three days under a nitrogen atmosphere and for three more days under vacuum, before storage in a glovebox. Analytical thin layer chromatography (TLC) was performed on Baker-Flex® Precoated Flexible TLC Sheets, impregnated with a fluorescent indicator (254 nm). Visualization of compounds on TLC plates was achieved by exposure to ultraviolet light. Column chromatography was executed using Fischer Chemical 40-63 μm, 230-400 mesh silica gel. Preparative gel-permeation chromatography (prep-GPC) was performed on a JAI Preparative Recycling HPLC (LaboACE-LC-5060) system equipped with 2.5HR and 2HR columns in series (20 mm ID×600 mm length) using chloroform as the eluent.

### General Materials Information

Unless otherwise noted, all reagents and starting materials were purchased from commercial vendors (Millipore-Sigma, Alfa Aesar, Strem, Ambeed, Beantown Chemical, Apollo Scientific, or Matrix Scientific) and used as received. Anhydrous THF, DMF, and toluene were purchased from Millipore-Sigma, packaged in Sure/Seal™ bottles, and used as received. Deuterated chloroform (CDCl₃) and deuterated dichloromethane (CD₂Cl₂) were purchased from Cambridge Isotope Laboratories (CIL). Deuterated toluene (Tol-d8) was purchased from either CIL or Millipore-Sigma. It was dried over and distilled from CaH₂, degassed by four freeze-pump-thaw cycles, then stored in a glovebox over activated 4 Å molecular sieves. Methyl methacrylate (MMA) was purchased from Millipore-Sigma. It was dried and had its stabilizer removed by passing it through a column of activated alumina, then degassed by four freeze-pump-thaw cycles, and stored in the dark in a freezer inside a glovebox; it was used within 1 month. ¹H NMR after 1 month of storage indicated no observable loss of quality. 1,1′-Azobis(cyclohexanecarbonitrile) (ACHN) was recrystallized from absolute ethanol as colorless crystals and were stored either in a fridge (˜2° C.) or a freezer inside a glovebox.

### General Analytical Information

NMR spectra were recorded on either Bruker AVANCE III DRX 400 or Neo 500 spectrometers at room temperature. Proton (¹H) chemical shifts are indicated in ppm and were calibrated to residual solvent peaks (CDCl₃: δ 7.26 ppm, CD₂Cl₂: δ 5.32 ppm, Tol-d8: δ 2.08 ppm). All carbon (¹³C) NMR recordings were proton-decoupled and their chemical shifts are also shown in ppm, referenced to the solvent's carbon resonance (CDCl₃: δ 77.16 ppm). Fluorine (¹⁹F) chemical shifts are also shown in ppm but are not referenced to a particular resonance. All NMR data were analyzed and processed using MestReNOVA. Quantitative ¹H NMR spectroscopy was performed using 1,4-bis(trimethylsilyl)benzene, 1,1,2,2-tetrachloroethane, or dibromomethane as an internal standard. High-resolution mass spectra were recorded on JEOL AccuTOF 4G equipped with an ionSense DART. Analytical size exclusion chromatography (SEC) was performed on a Tosoh EcoSEC HLC-8320 with dual TSKgel SuperH3000 columns and an ethanol-stabilized chloroform mobile phase. Samples were filtered through 0.2 m PTFE syringe filters before injection into the instrument. Molar mass values were calculated according to linear polymethylmethacrylate calibration standards. Thermal Gravimetric Analysis (TGA) studies were performed on samples of approximately 2-5 mg. The analyses were conducted using a TGA/DSC 2 STAR System (Mettler-Toledo) equipped with a Gas Controller GC 200 Star System. The studies were carried out with a temperature ramp of 10° C./minute. Differential Scanning Calorimetry (DSC) analyses were conducted using a TGA/DSC 2 STAR System (Mettler-Toledo), equipped with an RCS1-3277 DSC cell and a DSC1-0107 cooling system. Each sample, weighing about 3-6 mg, was sealed in an aluminum pan and subjected to three heating/cooling cycles ranging from 40° C. to 140° C. at a rate of 10° C./minute. The glass transition temperatures (T_{g}) were recorded from the second heating ramp. DSC traces from the second and third heating cycles were identical for all samples reported in this example. Rectangular bars of vPMMA, dPMMA(2.5), and dPMMA(5.0) for DMA were prepared through compression molding. Circular samples were created by iteratively filling a disk with a diameter of 30 mm and a thickness of 0.51 mm with pulverized polymer and pressing it at 5 tons of pressure and 135° C. for 30 minutes. This process was repeated three times to produce a transparent circular polymer disk. Rectangular bars were then cut from this disk and sanded to achieve uniformity. DMA was performed on a Discovery DMA 850 System (TA). Samples were tested in tensile mode. Measurements were recorded at a frequency of 1.0 Hz and an amplitude of 1.0 m from 40-150° C. at a ramp rate of 2° C./minute with a data sampling interval of 3 s/pt, using a 125% force tracking and 0.01 N preload force. Data were collected using Trios software and exported to Microsoft Excel for analysis.

### Simulation Details

### Generation of Contour Plots (FIG. 17A and FIG. 21)

Monte Carlo copolymerization simulations were conducted, targeting an average chain length of 1000 monomers and simulating a total of 4000 chains. To generate the contour plots, 625 pairs of reactivity ratios were simulated. This covered all combinations of 25 different r_{M} values, chosen at equal intervals in logspace(−1, 1), and r_{CC} values, chosen at equal intervals in logspace(−2, 2) for FIG. 17A or logspace (−3, 3) for FIG. 21. The data were interpolated to create a gradient illustration.

For each combination of reactivity ratios, a Monte Carlo simulation of the copolymerization process was conducted based on the Mayo-Lewis model. This involved generating a polymer array to represent the composition of each polymer chain. The chains were initialized with a monomer chosen based on the initial monomer amounts. The type of addition to occur was randomly selected based on the cumulative sum of propagation probabilities, which was calculated by considering the current amounts of monomers and the reactivity ratios. Depending on the reaction type, a polymer chain was randomly chosen for propagation. The selected chain was extended by adding either M or CC. After each addition, the length, molecular weight, and terminal monomer identity of the chain, and the remaining amount of each monomer were updated. The propagation probabilities were recalculated to reflect the new composition of the monomer pool and the current terminal monomer of each chain. The simulation continued until the predetermined number of monomers had reacted. For each polymer chain in the array, the runs of monomers (repeated elements), their lengths, and their values were analyzed using a predefined function. The weight-average molecular weight of the nondegradable segments was calculated based on the lengths of consecutive Ms.

Using the surfc function, a surface plot was generated to represent the decrease in polymer molecular weight after degradation across different combinations of reactivity ratios. For enhanced visualization, the axes were set to a logarithmic scale.

### Generation of Linear Sequences (FIG. 17B)

For each pair of reactivity ratios, a Monte Carlo simulation of the copolymerization process was conducted based on the Mayo-Lewis model. A polymer array was generated to represent a polymer chain. The chain was initialized with a monomer chosen based on the initial monomer amounts. The type of reaction to occur was randomly selected based on the cumulative sum of propagation probabilities, which was calculated by considering the current amounts of monomers and the reactivity ratios. Depending on the reaction type, the chain was extended by adding either M or CC. After each addition, the remaining amount of each monomer was updated. The propagation probabilities were recalculated to reflect the new composition of the monomer pool and the current terminal monomer of the chain. The simulation continued until the predetermined number of monomers had reacted.

### Computational Details

### General Computational Information

All Density Functional Theory (DFT) calculations were carried out using ORCA 5.0.4.¹⁰⁸ Geometry optimizations were performed using Head-Gordon's wB97X hybrid functional,¹⁰⁹ supplemented with Grimme's D3 dispersion correction (wB97X-D3).¹¹⁰ All intermediate and transition state geometries were optimized using the def2-SVP basis set.^{111,112} The Resolution of Identity and Chain-of-Spheres (RIJCOSX) approximation was applied,^{113,114} as implemented in ORCA 5.0.4, in conjunction with an auxiliary basis set, def2/J, to accelerate the calculations without a noticeable compromise in accuracy. Energies of the optimized geometries were reevaluated through single-point calculations using a triple-(quality basis set, def2-TZVP. Harmonic vibrational frequencies were computed at the wB97X/def2-SVP level of theory to ensure proper convergence to well-defined minima for ground states or first-order saddle points for transition states. Zero-point energy (ZPE) and thermal vibrational corrections were derived from these vibrational frequency calculations, with the latter specifically determined at a temperature of 373K. Solvation effects were accounted for using the Conductor-like Polarizable Continuum Model (CPCM)^{115,116} based on the optimized gas-phase geometries. A dielectric constant of 2.4 and a refractive index of 1.497 for toluene were incorporated into the calculations.

TABLE 1
Summary of Computed Energy Components

| | E_el/(kcal/mol) | ZPE/(kcal/mol) | non-ZPE vib/(kcal/mol) | |
|---|---|---|---|---|
| | def2-TZVP, CPCM(Tol) | def2-SVP | def2-SVP, T = 373K | G_solv/(kcal/mol) |
| MMA | −217011.2796 | 78.18 | 4.99 | −216928.1096 |
| 1 | −217377.6753 | 84.05 | 5.78 | −217287.8453 |
| M1 | −434411.423 | 165.65 | 13 | −434232.773 |
| TS-M1 | −434385.7071 | 163.44 | 13.33 | −434208.9371 |
| DOT | −635652.7453 | 128.13 | 9.03 | −635515.5853 |
| MeDOT | −660327.9664 | 145.59 | 10.43 | −660171.9464 |
| Me₂DOT | −684997.9265 | 162.97 | 11.8 | −684823.1565 |
| PhDOT | −780648.3448 | 179.35 | 13.53 | −780455.4648 |
| D1 for DOT | −853042.1541 | 214.34 | 17.5 | −852810.3141 |
| D1 for MeDOT | −877717.2748 | 232.03 | 18.83 | −877466.4148 |
| D1 for Me₂DOT | −902388.1159 | 249.29 | 20.25 | −902118.5759 |
| D1 for PhDOT | −998038.0908 | 265.39 | 22.15 | −997750.5508 |
| D2 for DOT | −853038.4696 | 211.41 | 18.72 | −852808.3396 |
| D2 for MeDOT | −877717.2644 | 232.05 | 18.82 | −877466.3944 |
| D2 for Me₂DOT | −902392.7139 | 247.21 | 21.42 | −902124.0839 |
| D2 for PhDOT | −998048.2099 | 263.67 | 22.87 | −997761.6699 |
| TS-D1 for DOT | −853027.9183 | 213.07 | 17.71 | −852797.1383 |
| TS-D1 for MeDOT | −877703.1711 | 230.33 | 19.18 | −877453.6611 |
| TS-D1 for Me₂DOT | −902370.5572 | 248.2 | 20.26 | −902102.0972 |
| TS-D1 for PhDOT | −998025.694 | 264.11 | 22.26 | −997739.324 |
| TS-D2 for DOT | −853018.7575 | 212 | 17.72 | −852789.0375 |
| TS-D2 for MeDOT | −877695.5438 | 229.82 | 19.14 | −877446.5838 |
| TS-D2 for Me₂DOT | −902370.5572 | 247.56 | 20.32 | −902102.6772 |
| TS-D2 for PhDOT | −998022.769 | 263.73 | 21.65 | −997737.389 |
| DM1 | −1070084.223 | 293.64 | 26.17 | −1069764.413 |
| TS-DM1 | −1070053.616 | 291.15 | 26.35 | −1069736.116 |
| MM1 | −651439.5093 | 246.8 | 19.86 | −661172.8493 |
| TS-MM1 | −651418.3992 | 244.93 | 20.74 | −651152.7292 |

### Synthesis and Characterization of Monomers

### Summary of Synthesis

FIGS. 25-30 summarize the synthesis of MeDOT and PhDOT (FIG. 25), Me₂DOT (FIG. 26), p-MePhDOT (FIG. 27), p-CF₃PhDOT (FIG. 28), F-p-CF₃PhDOT (FIG. 29), and PFPhDOT (FIG. 30).

### Synthetic Procedures

### Synthesis and Characterization of 2′-vinyl-[1,1′-biphenyl]-2-carbaldehyde (S3)

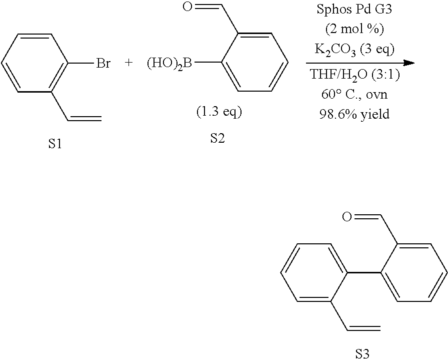

The following is a modified version of the procedure reported by the Buchwald group.¹¹⁷ In an oven-dried 250 mL round-bottom flask equipped with a magnetic stir bar were sequentially added Sphos Pd G3 (780 mg, 1 mmol, 0.02 equiv), 2-formyl phenylboronic acid (9.75 g, 65 mmol, 1.3 equiv), and K₂CO₃ (20.7 g, 150 mmol, 3.0 equiv) inside a nitrogen-filled glovebox. The flask was tightly sealed with a rubber septum and removed from the glovebox. Separately, a mixture of THF (90 mL) and water (30 mL) was purged with argon for 40 minutes in a Schlenk flask to remove oxygen. This mixture was then transferred into the round-bottom flask via cannula transfer. Next, 2-bromostyrene (6.3 mL, 50 mmol, 1.0 equiv) was added to the flask via syringe under a nitrogen flow. The resulting mixture was stirred vigorously in an oil bath preheated to 60° C. overnight. Afterward, the flask was removed from the oil bath and allowed to cool to room temperature. The reaction mixture was then filtered through a silica pad, which was washed with additional ethyl acetate to elute the product. The combined filtrate was dried over Na₂SO₄ and concentrated under reduced pressure using a rotary evaporator. The crude residue was purified by column chromatography on silica gel using EA:Hex=1:30 as the eluent. The product was obtained as a pale-yellow oil, which solidified into a waxy form after being placed under vacuum overnight (10.27 g, 98.6% yield).

¹H NMR (400 MHz, CDCl₃) δ 9.72 (d, J=0.8 Hz, 1H), 8.04 (dd, J=7.8, 1.4 Hz, 1H), 7.66 (ddd, J=16.8, 7.7, 1.4 Hz, 2H), 7.56-7.48 (m, 1H), 7.43 (td, J=7.6, 1.5 Hz, 1H), 7.40-7.31 (m, 2H), 7.28-7.22 (m, 1H), 6.41 (dd, J=17.5, 11.0 Hz, 1H), 5.68 (dd, J=17.4, 1.1 Hz, 1H), 5.17 (dd, J=11.0, 1.1 Hz, 1H).

¹³C{¹H}NMR (101 MHz, CDCl₃) δ 192.24, 144.67, 137.09, 136.45, 134.78, 134.32, 133.68, 131.35, 130.81, 128.66, 128.15, 127.67, 127.20, 125.45, 116.40.

HRMS (DART-TOF) C₁₅H₁₂O [M+H]⁺ calcd: 209.09609 found: 209.09623.

### Synthesis and Characterization of 7-methyldibenzo[c,e]oxepin-5(7H)-one (S6, MeDOO)

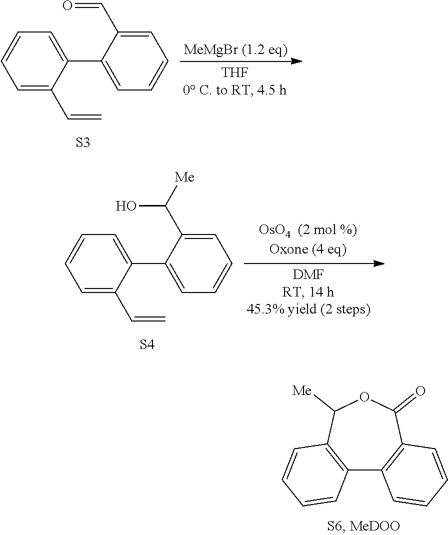

To a 40 mL scintillation vial equipped with a magnetic stir bar was added 2′-vinyl-[1,1′-biphenyl]-2-carbaldehyde (S3, 1.5 g, 7.2 mmol, 1.0 equiv). The vial was sealed with a penetrable cap, then evacuated and backfilled with nitrogen four times. Anhydrous THF (24 mL) was then added against a nitrogen flow, after which the vial was cooled in an ice bath. A 3 M solution of MeMgBr in Et₂O (2.88 mL, 8.64 mmol, 1.2 equiv) was added dropwise into the vial at 0° C. Upon addition, a white insoluble solid formed in the vial. The resulting mixture was stirred at 0° C. for an additional 30 minutes and then at room temperature for 4 hours. The insoluble solid disappeared upon warming to room temperature, and the mixture turned yellow. The reaction was quenched by the addition of water (2 mL), followed by a saturated aqueous solution of NH₄Cl (2 mL). The mixture was then transferred to a separatory funnel, to which additional saturated NH₄Cl solution (60 mL) was added, and subsequently extracted three times with EtOAc (3×50 mL). The combined organic layers were dried over Na₂SO₄ and concentrated under reduced pressure using a rotary evaporator. The resulting pale-yellow oil was used for the subsequent reaction without further purification.

The pale-yellow oil was transferred to a 40 mL scintillation vial and kept under vacuum overnight. After the addition of Oxone (8.85 g, 28.8 mmol, 4.0 equiv), the vial was sealed with a penetrable cap, evacuated, and backfilled with nitrogen four times. Anhydrous DMF (8 mL) was then introduced into the vial against a nitrogen flow. The resulting mixture was vortexed for one minute. Subsequently, a 2.5 wt % OsO₄ solution in t-BuOH (1.875 mL, 0.144 mmol, 0.02 equiv) was added dropwise to the vial while stirring vigorously. The mixture turned into a brown-black slurry and generated heat. It was allowed to stir at room temperature for 14 hours. Water (10 mL) was then added, and the mixture was transferred to a separatory funnel. Brine (50 mL) was added, followed by extractions with EtOAc (3×40 mL). The combined organic layers were washed thoroughly with an aqueous solution of Na₂SO₃ (2×80 mL) to quench residual OsO₄ and then washed with brine (2×80 mL) to remove residual DMF. The mixture was dried over Na₂SO₄ and concentrated under reduced pressure using a rotary evaporator. The crude residue was purified by column chromatography on silica gel using EA:Hex=1:7 as the eluent. The product was obtained as a white solid (731.6 mg, 45.3% yield over 2 steps).

¹H NMR (400 MHz, CDCl₃) δ 7.98 (dd, J=7.8, 1.4 Hz, 1H), 7.66 (td, J=7.6, 1.4 Hz, 1H), 7.59 (td, J=6.2, 3.2 Hz, 2H), 7.51 (dddd, J=18.7, 12.9, 7.4, 1.6 Hz, 4H), 5.28 (q, J=6.6 Hz, 1H), 1.84 (d, J=6.6 Hz, 3H).

¹³C{¹H}NMR (101 MHz, CDCl₃) δ 170.04, 138.71, 137.60, 137.40, 132.60, 131.36, 130.94, 129.64, 129.02, 128.88, 128.63, 128.43, 124.00, 73.18, 16.94.

HRMS (DART-TOF) C₁₅H₁₂O₂[M+H]⁺ calcd: 225.09101 found: 225.09131.

### Synthesis and Characterization of 7-methyldibenzo[c,e]oxepine-5(7H)-thione (S6, MeDOT)

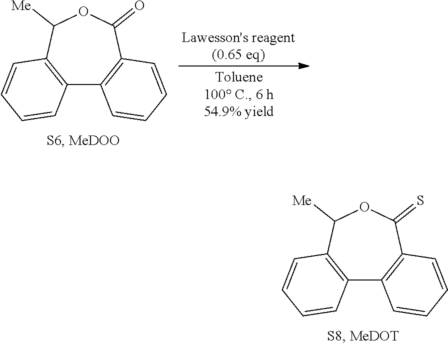 [SMILES: CC1OC(=O)c2ccccc2-c2ccccc21 ; CC1OC(=S)c2ccccc2-c2ccccc21]

To a 20 mL scintillation vial equipped with a magnetic stir bar were added 7-methyldibenzo[c,e]oxepin-5(7H)-one (S6, 731.6 mg, 3.26 mmol, 1.0 equiv) and Lawesson's reagent (857.7 mg, 2.12 mmol, 0.65 equiv). The vial was sealed with a penetrable cap and evacuated, then backfilled with nitrogen four times. Anhydrous toluene (6 mL) was added via syringe, and the mixture was stirred at 100° C. for 6 hours, during which the color turned orange. The mixture was allowed to cool to room temperature and filtered through a short pad of silica gel. The pad was washed with additional EtOAc to collect the yellow product. The combined filtrates were concentrated under reduced pressure using a rotary evaporator and kept under vacuum for several hours to remove residual solvent. The crude residue was purified by column chromatography on silica gel, using a gradient from EA:Hex=1:30 to 1:20 (R_{f}=0.25 for EA:Hex=1:20). The product was obtained as a yellow solid (430.2 mg, 54.9% yield).

¹H NMR (400 MHz, CDCl₃) δ 8.18 (dd, J=7.9, 1.4 Hz, 1H), 7.66-7.58 (m, 2H), 7.58-7.42 (m, 5H), 5.41 (q, J=6.6 Hz, 1H), 1.96 (d, J=6.6 Hz, 3H).

¹³C{¹H}NMR (101 MHz, CDCl₃) δ 215.86, 139.59, 138.87, 137.35, 134.79, 133.47, 132.10, 129.93, 128.88, 128.79, 128.76, 128.20, 123.93, 78.74, 16.84.

HRMS (DART-TOF) C₁₅H₁₂OS [M+H]⁺ calcd: 241.06816 found: 241.06874.

### Synthesis and Characterization of 7-phenyldibenzo[c,e]oxepin-5(7H)-one (S7, PhDOO)

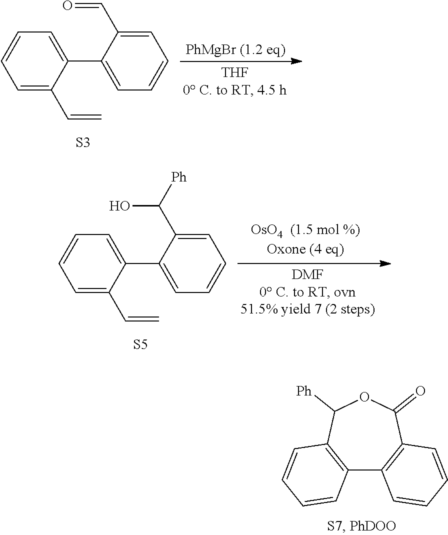

2′-Vinyl-[1,1′-biphenyl]-2-carbaldehyde (S3, 1.451 g, 6.97 mmol, 1.0 equiv) was weighed into a 40 mL scintillation vial equipped with a magnetic stir bar. The vial was sealed with a penetrable cap, then evacuated and backfilled with nitrogen four times. Anhydrous THF (23 mL) was added against a nitrogen flow to completely dissolve the starting material. The mixture was cooled to 0° C. in an ice bath. A 1.6 M solution of phenyl magnesium bromide in cyclopentyl methyl ether (5.23 mL, 8.36 mmol, 1.2 equiv) was added dropwise under stirring. The resulting mixture was stirred at 0° C. for an additional 30 minutes and then allowed to stir at room temperature for 4 hours. The reaction was quenched by the addition of water (5 mL), followed by a saturated aqueous solution of NH₄Cl (60 mL). The aqueous layer was extracted with EtOAc (3×50 mL). The combined organic layers were dried over Na₂SO₄ and concentrated under reduced pressure using a rotary evaporator. The crude residue was purified by column chromatography on silica gel using EA:Hex=1:8 as the eluent (R_{f}=0.20-0.34 for EA:Hex=1:7). All fractions containing the product were collected and the solvent was removed. The resulting colorless oil was used for the subsequent reaction.

The colorless oil was transferred into a 40 mL scintillation vial and kept under vacuum overnight. After the addition of Oxone (7.98 g, 26.0 mmol, 4.0 equiv), the vial was sealed with a penetrable cap and charged with nitrogen. Anhydrous DMF (20 mL) was added via syringe against a nitrogen flow, and the mixture was stirred vigorously until the colorless oil fully homogenized with the solvent. The mixture was then cooled to 0° C. in an ice bath. A 2.5 wt % OsO₄ solution in t-BuOH (1.27 mL, 0.097 mmol, 0.015 equiv) was added dropwise into the vial at 0° C. The resulting mixture was allowed to warm slowly to room temperature and stirred for 13 hours, turning yellow in the process. Water (10 mL) was added, and the mixture was transferred to a separatory funnel. Brine (50 mL) was then added, and the mixture was extracted with EtOAc (3×40 mL). The combined organic layers were washed thoroughly with an aqueous solution of Na₂SO₃ (2×80 mL) to quench residual OsO₄ and then washed with brine (2×80 mL) to remove residual DMF. The mixture was dried over Na₂SO₄ and concentrated under reduced pressure using a rotary evaporator. The crude residue was purified by column chromatography on silica gel, using a gradient from EA:Hex=1:10 to EA:Hex=1:7 (R_{f}=0.28 for EA:Hex=1:7). The product was obtained as a foamy white solid (857.6 mg, 43.0% yield over 2 steps).

¹H NMR (400 MHz, CDCl₃) δ 8.03 (d, J=7.8 Hz, 1H), 7.70 (ddd, J=20.6, 14.0, 7.6 Hz, 3H), 7.61-7.54 (m, 1H), 7.54-7.39 (m, 6H), 7.29 (t, J=7.6 Hz, 1H), 6.80 (d, J=7.8 Hz, 1H), 6.25 (s, 1H).

¹³C{¹H}NMR (101 MHz, CDCl₃) δ 169.56, 138.74, 138.64, 137.47, 135.92, 132.85, 131.63, 130.93, 129.71, 129.07, 128.95, 128.72, 128.69, 128.62, 128.57, 127.56, 127.14, 79.16.

HRMS (DART-TOF) C₂₀H₁₄O₂[M+H]⁺ calcd: 287.10666 found: 287.10901.

### Synthesis and Characterization of 7-phenyldibenzo[c,e]oxepine-5(7H)-thione (S9, PhDOT)

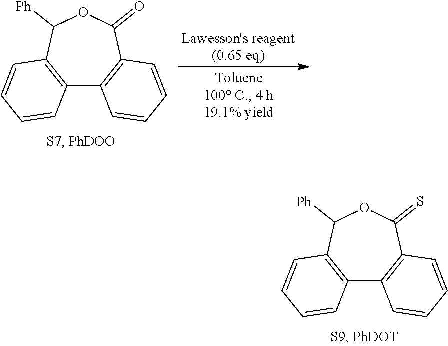 [SMILES: O=C1OC(c2ccccc2)c2ccccc2-c2ccccc21 ; S=C1OC(c2ccccc2)c2ccccc2-c2ccccc21]

To a 40 mL scintillation vial equipped with a magnetic stir bar were added 7-phenyldibenzo[c,e]oxepin-5(7H)-one (S7, 857.6 mg, 3.00 mmol, 1.0 equiv) and Lawesson's reagent (787.4 mg, 1.95 mmol, 0.65 equiv). The vial was sealed with a penetrable cap, then evacuated and backfilled with nitrogen four times. After adding anhydrous toluene (5 mL), the mixture was stirred at 100° C. for 4 hours until the color of the mixture turned orange. The mixture was allowed to cool to room temperature and then filtered through a short pad of silica gel. The pad was washed with additional EtOAc to elute and collect the yellow product. The combined filtrates were concentrated under reduced pressure using a rotary evaporator and were kept under vacuum for a few hours to remove residual solvent. The crude residue was purified by column chromatography on silica gel, using a gradient from EA:Hex=1:40 to 1:30 (R_{f}=0.25 for EA:Hex=1:20). All fractions containing the product were collected. The unreacted starting material was recovered (342 mg, 39.9% recovery yield) from the column using EA:Hex=1:7 as the eluent (R_{f}=0.13 for EA:Hex=1:20). This recovered material was resubjected to the same thionation condition using Lawesson's reagent (289.9 mg, 0.717 mmol, 0.6 equiv) and toluene (4 mL) as the solvent. The resulting crude mixture was purified following the aforementioned workup procedure and a column chromatography using EA:Hex=1:30 as the eluent. Fractions containing the desired product were collected and combined with those from the first column. The combined fractions were concentrated under reduced pressure using a rotary evaporator and subjected to another round of column chromatography on silica gel, using a gradient from Hex:DCM=4:1 to Hex:DCM=2:1 (R_{f}=0.28 for Hex:DCM=2:1). The product was obtained as a yellow crystalline solid (175 mg, 19.1% overall yield).

¹H NMR (400 MHz, CDCl₃) δ 8.22 (dd, J=7.9, 1.4 Hz, 1H), 7.67 (ddd, J=8.9, 4.8, 2.6 Hz, 2H), 7.59 (dt, J=7.4, 1.8 Hz, 3H), 7.54-7.42 (m, 5H), 7.31 (td, J=7.6, 1.2 Hz, 1H), 6.84 (d, J=7.8 Hz, 1H), 6.35 (s, 1H).

¹³C{¹H}NMR (101 MHz, CDCl₃) δ 214.88, 139.49, 138.74, 138.42, 135.23, 134.75, 133.66, 132.25, 129.88, 128.91, 128.86, 128.79, 128.73, 128.65, 128.38, 127.74, 126.90, 84.30.

HRMS (DART-TOF) C₂₀H₁₄OS [M+H]⁺ calcd: 303.08381 found: 303.08413.

### Synthesis and Characterization of methyl 2′-vinyl-[1,1′-biphenyl]-2-carboxylate (S11)

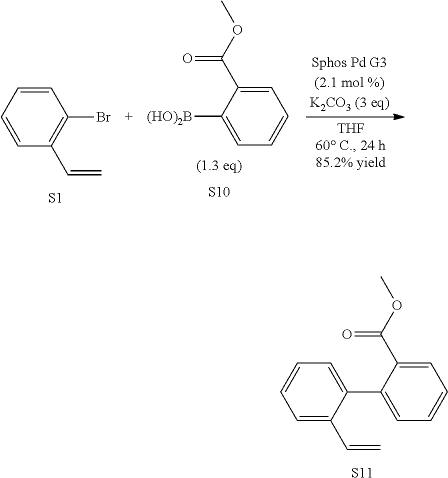

To an oven-dried 250 mL round-bottom flask were sequentially added Sphos Pd G3 (312.1 mg, 0.4 mmol, 0.021 equiv), 2-methoxycarbonyl phenylboronic acid (4.38 g, 24.34 mmol, 1.3 equiv), and K₂CO₃ (7.76 g, 56.16 mmol, 3.0 equiv) inside a nitrogen-filled glovebox. The flask was then tightly sealed with a rubber septum and removed from the glovebox. Anhydrous THF (45 mL) and 2-bromostyrene (2.35 mL, 18.72 mmol, 1.0 equiv) were added via syringe to the flask under a nitrogen atmosphere. The resulting mixture was stirred vigorously in a preheated oil bath at 60° C. for 24 hours. Afterward, the flask was removed from the oil bath and allowed to cool to room temperature. The resulting grey slurry was filtered through a pad of silica gel, which was subsequently washed with additional ethyl acetate to elute the product. The combined filtrate was dried over Na₂SO₄ and concentrated under reduced pressure using a rotary evaporator. The crude residue was then purified by column chromatography on silica gel, using EA:Hex=1:30 as the eluent (R_{f}=0.22 for EA:Hex=1:30). The product was obtained as a colorless oil (3.80 g, 85.2% yield).

¹H NMR (400 MHz, CDCl₃) δ 7.98 (dd, J=7.8, 1.4 Hz, 1H), 7.65 (dd, J=7.8, 1.5 Hz, 1H), 7.57 (td, J=7.5, 1.4 Hz, 1H), 7.46 (td, J=7.6, 1.3 Hz, 1H), 7.42-7.25 (m, 3H), 7.17 (dd, J=7.4, 1.6 Hz, 1H), 6.46 (dd, J=17.5, 11.0 Hz, 1H), 5.66 (dd, J=17.4, 1.3 Hz, 1H), 5.13 (dd, J=11.0, 1.2 Hz, 1H), 3.61 (s, 3H).

¹³C{¹H}NMR (101 MHz, CDCl₃) δ 167.97, 141.94, 140.51, 135.82, 135.22, 131.61, 131.58, 131.15, 130.07, 129.23, 127.62, 127.47, 127.35, 124.82, 114.93, 52.09.

HRMS (DART-TOF) C₁₆H₁₄O₂[M+H]⁺ calcd: 239.10666 found: 239.10659.

### Synthesis and Characterization of 2-(2′-vinyl-[1,1′-biphenyl]-2-yl)propan-2-ol (S12)

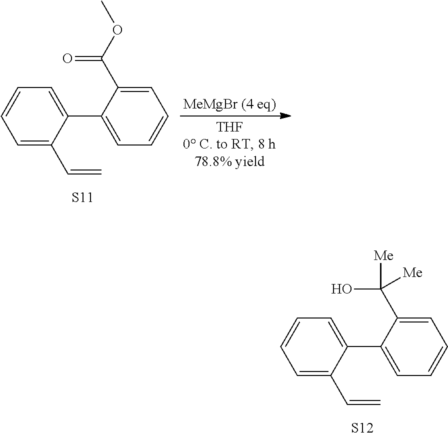 [SMILES: C=Cc1ccccc1-c1ccccc1C(=O)OC ; "S11" ; C=Cc1ccccc1-c1ccccc1C(C)(C)O ; "S12"]

Methyl 2′-vinyl-[1,1′-biphenyl]-2-carboxylate (S11, 3.25 g, 13.6 mmol, 1.0 equiv) was weighed into a flame-dried 250 mL round-bottom flask equipped with a magnetic stir bar. The flask was sealed with a rubber septum and then evacuated and backfilled with nitrogen four times. Anhydrous THF (48 mL) was added to the flask against a nitrogen flow. The flask was placed in an ice bath, and a 3 M solution of MeMgBr in diethyl ether (18.2 mL, 54.6 mmol, 4.0 equiv) was added dropwise via syringe at 0° C. The resulting light grey mixture was stirred at 0° C. for an additional 30 minutes, and then at room temperature for 7 hours and 30 minutes. To quench the reaction, water (10 mL) and a saturated solution of NH₄Cl (20 mL) were added to the resulting yellow solution. The THF was evaporated to a minimal volume under reduced pressure using a rotary evaporator. The mixture was then transferred to a separatory funnel and further extracted with EtOAc (3×100 mL), following the addition of a saturated aqueous solution of NH₄Cl (80 mL). The combined organic layers were dried over Na₂SO₄ and subsequently concentrated using a rotary evaporator. The crude residue was purified by column chromatography on silica gel, using a gradient from EA:Hex=1:10 to 1:8 (R_{f}=0.33 for EA:Hex=1:7). The product was obtained as a white waxy solid (2.56 g, 78.8% yield).

¹H NMR (400 MHz, CDCl₃) δ 7.70 (dd, J=8.0, 1.3 Hz, 2H), 7.46-7.35 (m, 2H), 7.35-7.20 (m, 3H), 7.02 (dd, J=7.5, 1.5 Hz, 1H), 6.43 (dd, J=17.6, 11.0 Hz, 1H), 5.68 (dd, J=17.6, 1.2 Hz, 1H), 5.14 (dd, J=11.0, 1.2 Hz, 1H), 1.77 (s, 1H), 1.46 (d, J=28.0 Hz, 6H).

¹³C{¹H}NMR (101 MHz, CDCl₃) δ 146.60, 142.26, 138.00, 136.24, 135.62, 132.20, 130.33, 127.74, 127.66, 127.04, 126.36, 126.32, 124.87, 114.68, 74.12, 32.42, 32.14.

HRMS (DART-TOF) C₁₇H₁₈O [M+H-H₂O]⁺ calcd: 221.13248 found: 221.13108.

### Synthesis and Characterization of 7,7-dimethyldibenzo[c,e]oxepin-5(7H)-one (S13, Me₂DOO)

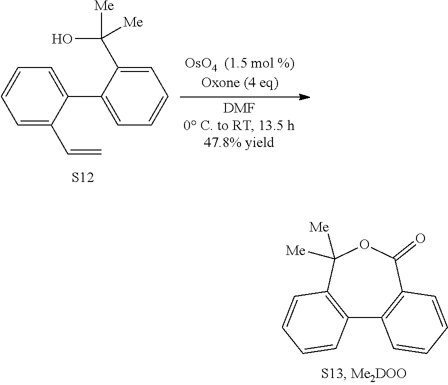 [SMILES: C=Cc1ccccc1-c1ccccc1C(C)(C)O ; "S12" ; CC1(C)OC(=O)c2ccccc2-c2ccccc21]

To a flame-dried 100 mL round-bottom flask were added 2-(2′-vinyl-[1,1′-biphenyl]-2-yl)propan-2-ol (S12, 2.56 g, 10.7 mmol, 1.0 equiv) and Oxone (13.2 g, 43.0 mmol, 4.0 equiv). The flask was sealed with a rubber septum, then evacuated and backfilled with nitrogen four times. Anhydrous DMF (30 mL) was subsequently added, and the mixture was cooled to 0° C. in an ice bath while being stirred vigorously. A 2.5 wt % OsO₄ solution in t-BuOH (2.1 mL, 0.16 mmol, 0.015 equiv) was added dropwise to the flask at 0° C. The resulting black mixture was stirred at 0° C. for an additional 30 minutes before being allowed to slowly warm to room temperature. The mixture was stirred vigorously for 13 hours at room temperature, during which it eventually turned yellow. After addition of water (30 mL), the mixture was transferred to a separatory funnel. Brine was then added (90 mL), and the mixture was extracted with EtOAc (3×80 mL). The combined organic layers were washed thoroughly with an aqueous solution of Na₂SO₃ (2×160 mL) to quench residual OsO₄ and then washed with brine (2×160 mL) to remove residual DMF. The mixture was dried over Na₂SO₄ and concentrated under reduced pressure using a rotary evaporator. The crude residue was purified by column chromatography on silica gel, using a gradient from EA:Hex=1:10 to 1:7. The product was obtained as a white solid (1.224 g, 47.8%).

¹H NMR (400 MHz, CDCl₃) δ 7.97 (dd, J=7.7, 1.4 Hz, 1H), 7.66 (td, J=7.6, 1.5 Hz, 1H), 7.57 (dd, J=7.4, 1.4 Hz, 2H), 7.54 (dd, J=7.7, 1.4 Hz, 1H), 7.51-7.45 (m, 2H), 7.42 (td, J=7.6, 1.5 Hz, 1H), 1.70 (s, 6H).

¹³C{¹H}NMR (101 MHz, CDCl₃) δ 169.28, 141.52, 139.01, 137.44, 132.93, 131.65, 131.61, 130.92, 129.35, 129.20, 128.57, 128.27, 125.15, 81.82.

HRMS (DART-TOF) C₁₆H₁₄O₂[M+H]⁺ calcd: 239.10666 found: 239.10696.

### Synthesis and Characterization of 7,7-dimethyldibenzo[c,e]oxepine-5(7H)-thione (S14, M e₂DOT)

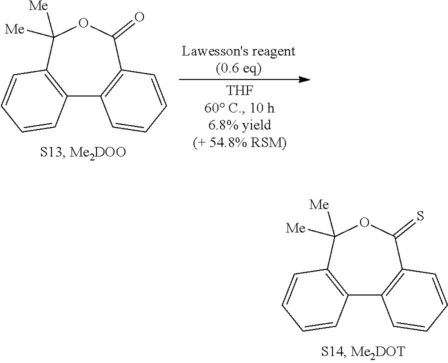 [SMILES: CC1(C)OC(=O)c2ccccc2-c2ccccc21 ; CC1(C)OC(=S)c2ccccc2-c2ccccc21]

To a 40 mL scintillation vial equipped with a magnetic stir bar were added 7,7-dimethyldibenzo[c,e]oxepin-5(7H)-one (S13, 1.008 g, 4.23 mmol, 1.0 equiv) and Lawesson's reagent (1.027 g, 2.54 mmol, 0.6 equiv). The vial was sealed with a penetrable cap, then evacuated and backfilled with nitrogen four times. After adding anhydrous THF (14 mL), the mixture was stirred at 60° C. for 10 hours. The mixture turned yellow after 10 hours of reaction. The reaction mixture was allowed to cool to room temperature and then filtered through a short pad of silica gel. The pad was washed with additional EtOAc to elute and collect the yellow product. The combined filtrates were concentrated under reduced pressure using a rotary evaporator. The crude residue was purified by column chromatography on silica gel, using EA:Hex=1:20 as the eluent (R_{f}=0.41 for EA:Hex=1:7). All fractions containing the product were collected. The unreacted starting material was recovered (776.7 mg, 77.0% recovery yield) from the column using EA:Hex=1:7 as the eluent (R_{f}=0.24 for EA:Hex=1:7). This recovered material was resubjected to the same thionation condition using Lawesson's reagent (791 mg, 1.96 mmol, 0.6 equiv) and THF (10.5 mL) as the solvent. The resulting crude mixture was purified following the aforementioned workup procedure and column chromatography using EA:Hex=1:20 as the eluent. The unreacted starting material was again recovered (552.4 mg, 54.8% overall recovery yield) from the column using EA:Hex=1:7 as the eluent. Fractions containing the desired product were collected and combined with those from the first column. The combined fractions were concentrated under reduced pressure using a rotary evaporator. The residue was then subjected to another round of column chromatography on silica gel, using a gradient from Hex:DCM=4:1 to 2:1 (R_{f}=0.49 for Hex:DCM=1:1). The product was obtained as a yellow solid (72.8 mg, 6.8% overall yield).

¹H NMR (400 MHz, CDCl₃) δ 8.18 (dd, J=7.9, 1.4 Hz, 1H), 7.62-7.54 (m, 2H), 7.54-7.37 (m, 5H), 2.06 (s, 3H), 1.46 (s, 3H).

¹³C{¹H}NMR (101 MHz, CDCl₃) δ 215.77, 141.19, 139.60, 137.31, 136.04, 133.74, 132.28, 130.55, 129.49, 128.90, 128.56, 127.88, 124.89, 87.97, 29.78.

HRMS (DART-TOF) C₁₆H₁₄OS [M+H]⁺ calcd: 255.08381 found: 255.08401.

### Synthesis and Characterization of 7-(p-tolyl)dibenzo[c,e]oxepin-5(7H)-one (516, p-MePhDOO)

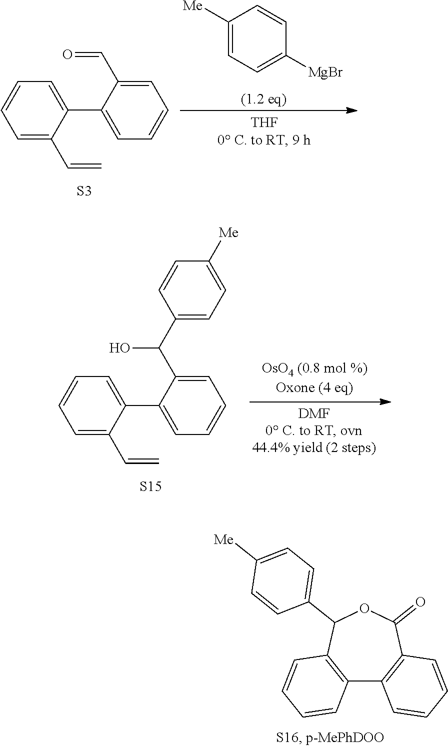

To a flame-dried 250 mL round-bottom flask equipped with a magnetic stir bar was added 2′-vinyl-[1,1′-biphenyl]-2-carbaldehyde (S3, 10.95 g, 52.6 mmol, 1.0 equiv). The flask was sealed with a rubber septum, then evacuated and backfilled with nitrogen four times. Anhydrous THF (80 mL) was added, and the mixture was stirred at room temperature until the starting material had fully dissolved. The mixture was then cooled to 0° C. in an ice bath. 0.5 M p-Tolylmagnesium bromide solution in Et₂O (126.2 mL, 63.1 mmol, 1.2 equiv) was added dropwise while stirring over a period of 30 minutes. The resulting mixture was allowed to stir at 0° C. for an additional 30 minutes, and then stirred at room temperature for 8 hours. The reaction was quenched by the addition of water (10 mL), followed by a saturated aqueous solution of NH₄Cl (150 mL). The aqueous layer was extracted with EtOAc (3×100 mL). The combined organic layers were dried over Na₂SO₄ and concentrated under reduced pressure using a rotary evaporator. The crude residue was purified by column chromatography on silica gel, using a gradient from EA:Hex=10:1 to 7:1. All fractions containing the product were collected and the solvent was removed. The resulting viscous, colorless oil was used for the subsequent reaction.

The viscous, colorless oil was transferred into a 250 mL round-bottom flask equipped with a magnetic stir bar and kept under vacuum overnight. After the addition of Oxone (64.6 g, 210.3 mmol, 4.0 equiv), the flask was sealed with a rubber septum and charged with nitrogen. Anhydrous DMF (79 mL) was added via syringe against a nitrogen flow, and the resulting mixture was stirred vigorously until the colorless oil had fully homogenized with the solvent. The mixture was then cooled to 0° C. in an ice bath. A 2.5 wt % OsO₄ solution in t-BuOH (5.48 mL, 0.42 mmol, 0.008 equiv) was added dropwise to the flask at 0° C. The resulting black mixture was stirred at 0° C. for an additional 30 minutes and then allowed to stir overnight at room temperature. The mixture eventually turned yellow. After the addition of water (50 mL), the mixture was transferred to a separatory funnel, brine (100 mL) was added, and the aqueous layer was extracted with EtOAc (3×100 mL). The combined organic layers were thoroughly washed with an aqueous solution of Na₂SO₃ (2×200 mL) to quench residual OsO₄ and then washed with water (2×200) and brine (2×200 mL) to remove residual DMF. The mixture was dried over Na₂SO₄ and then concentrated under reduced pressure using a rotary evaporator. The crude residue was purified by column chromatography on silica gel, using a gradient from EA:Hex=1:10 to 1:9. The product was obtained as a foamy white solid. (5.275 g, 33.4% yield over 2 steps)

¹H NMR (400 MHz, CDCl₃) δ 8.03 (d, J=7.7 Hz, 1H), 7.77-7.62 (m, 3H), 7.60-7.53 (m, 1H), 7.48 (t, J=7.6 Hz, 1H), 7.40 (d, J=7.7 Hz, 2H), 7.28 (d, J=7.8 Hz, 3H), 6.83 (d, J=7.8 Hz, 1H), 6.23 (s, 1H), 2.43 (s, 3H).

¹³C{¹H}NMR (101 MHz, CDCl₃) δ 169.61, 138.84, 138.53, 138.33, 137.43, 132.84, 132.77, 131.54, 130.92, 129.60, 129.31, 129.01, 128.86, 128.62, 128.49, 127.42, 127.10, 79.13, 21.35.

HRMS (DART-TOF) C₂₁H₁₆O₂[M+H]⁺ calcd: 301.12231 found: 301.12200.

### Synthesis and Characterization of 7-(p-tolyl)dibenzo[c,e]oxepine-5(7H)-thione (S17, p-MePhDOT)

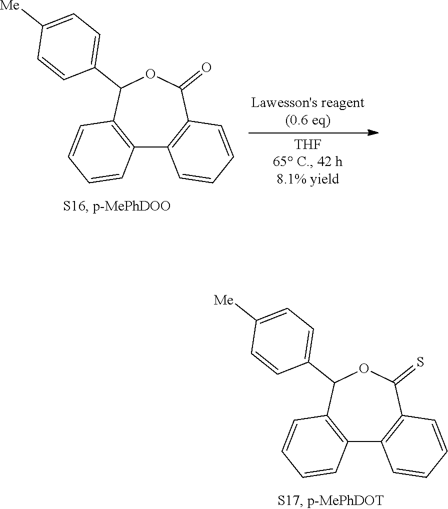 [SMILES: Cc1ccc(C2OC(=O)c3ccccc3-c3ccccc32)cc1 ; Cc1ccc(C2OC(=S)c3ccccc3-c3ccccc32)cc1]

To a flame-dried 250 mL round-bottom flask equipped with a magnetic stir bar were added 7-(p-tolyl)dibenzo[c,e]oxepin-5(7H)-one (S16, 5.275 g, 17.6 mmol, 1.0 equiv) and Lawesson's reagent (4.262 g, 10.5 mmol, 0.6 equiv). The flask was sealed with a rubber septum and purged with nitrogen. Anhydrous THF (40 mL) was then added, and the resulting mixture was stirred in an oil bath preheated to 65° C. for 42 hours. The flask was removed from the oil bath and allowed to cool to room temperature. The resulting mixture was then filtered through a pad of Celite, which was washed with additional EtOAc to fully elute the yellow-colored product. The combined filtrates were concentrated under reduced pressure using a rotary evaporator and kept under high vacuum for a few hours to ensure complete removal of the solvent. The crude residue was purified by column chromatography on silica gel, using a gradient from DCM:Hex=1:4 to 1:3. The product was isolated as a foamy yellow solid (451.0 mg, 8.1% yield).

¹H NMR (400 MHz, CDCl₃) δ 8.14 (dd, J=7.9, 1.3 Hz, 1H), 7.60 (ddd, J=8.7, 4.2, 2.8 Hz, 2H), 7.51 (d, J=8.2 Hz, 1H), 7.47-7.36 (m, 4H), 7.23 (q, J=10.2, 9.2 Hz, 3H), 6.79 (d, J=7.8 Hz, 1H), 6.23 (s, 1H), 2.36 (s, 3H).

¹³C{¹H}NMR (101 MHz, CDCl₃) δ 215.01, 139.52, 138.67, 138.57, 134.76, 133.61, 132.19, 132.18, 129.80, 129.44, 128.87, 128.68, 128.60, 128.32, 127.66, 126.91, 84.38, 21.42.

HRMS (DART-TOF) C₂₁H₁₆OS [M+H]⁺ calcd: 317.09946 found: 317.10037.

### Synthesis and Characterization of 7-(4-(trifluoromethyl)phenyl)dibenzo[c,e]oxepin-5(7H)-one (S19, p-CF₃PhDOO)

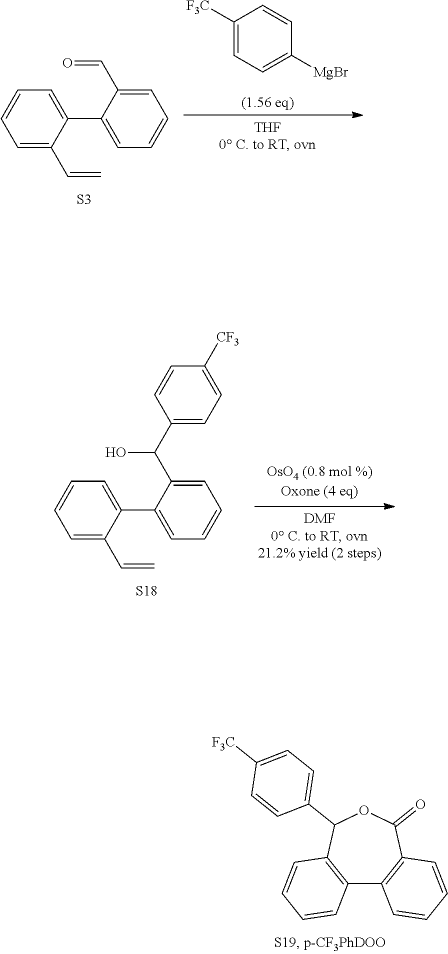

To a flame-dried 100 mL round-bottom flask, equipped with a magnetic stir bar, were added magnesium turnings (1.604 g, 66 mmol, 1.1 equiv) and LiCl (2.543 g, 60 mmol, 1.0 equiv) inside a nitrogen-filled glovebox. The flask was sealed with a rubber septum and then removed from the glovebox. Anhydrous Et₂O (60 mL) was added to the flask via syringe, against a flow of nitrogen. After adding a small portion of 4-bromobenzotrifluoride (0.2 mL out of 8.4 mL total), the mixture was stirred at 50° C. until the magnesium was activated and the color of the solution changed to brown. The mixture was subsequently cooled to 0° C., after which the remaining 4-bromobenzotrifluoride (8.2 mL out of the initial 8.4 mL, 60 mmol, 1.0 equiv) was added dropwise. The mixture was stirred for an additional 30 minutes at 0° C., followed by 1.5 hours at room temperature. The resulting dark brown solution of 1 M 4-(trifluoromethyl) phenylmagnesium bromide in Et₂O was used for the subsequent reaction.

To a flame-dried 250 mL round-bottom flask equipped with a magnetic stir bar was added 2′-vinyl-[1,1′-biphenyl]-2-carbaldehyde (S3, 8.0 g, 38.4 mmol, 1.0 equiv). The flask was sealed with a rubber septum, then evacuated and backfilled with nitrogen four times. Anhydrous THF (100 mL) was added via syringe, and the mixture was stirred at room temperature until the starting material had fully dissolved. The mixture was then cooled to 0° C. in an ice bath. 1M 4-(trifluoromethyl)phenylmagnesium bromide solution in Et₂O (60 mL, 60 mmol, 1.56 equiv) was added dropwise while stirring over a period of 30 minutes. The resulting mixture was allowed to stir at 0° C. for an additional 30 minutes, and then stirred at room temperature overnight. The reaction was quenched by the addition of water (10 mL), followed by a saturated aqueous solution of NH₄Cl (150 mL). The aqueous layer was extracted with EtOAc (3×100 mL). The combined organic layers were dried over Na₂SO₄ and concentrated under reduced pressure using a rotary evaporator. The crude residue was purified by column chromatography on silica gel, using a gradient from EA:Hex=1:10 to 1:8. All fractions containing the product were collected and the solvent was removed. The resulting viscous, colorless oil was used for the subsequent reaction.

The viscous, colorless oil was transferred into a 250 mL round-bottom flask equipped with a magnetic stir bar and kept under vacuum overnight. After the addition of Oxone (49.15 g, 160 mmol, 4.0 equiv), the flask was sealed with a rubber septum and charged with nitrogen. Anhydrous DMF (60 mL) was added via syringe against a nitrogen flow, and the resulting mixture was stirred vigorously until the colorless oil had fully homogenized with the solvent. The mixture was then cooled to 0° C. in an ice bath. A 2.5 wt % OsO₄ solution in t-BuOH (4.17 mL, 0.32 mmol, 0.008 equiv) was added dropwise to the flask at 0° C. The resulting black mixture was stirred at 0° C. for an additional 30 minutes and then allowed to stir overnight at room temperature. After the addition of water (50 mL), the mixture was transferred to a separatory funnel, brine (100 mL) was added, and the aqueous layer was extracted with EtOAc (3×100 mL). The combined organic layers were thoroughly washed with an aqueous solution of Na₂SO₃ (2×200 mL) to quench residual OsO₄ and then washed with water (2×200) and brine (2×200 mL) to remove residual DMF. The mixture was dried over Na₂SO₄ and then concentrated under reduced pressure using a rotary evaporator. The crude residue was purified by column chromatography on silica gel, using a gradient from EA:Hex=1:10 to 1:9. The product was obtained as a white solid (3.00 g, 21.2% yield over 2 steps).

¹H NMR (400 MHz, CDCl₃) δ 8.04 (d, J=7.8 Hz, 1H), 7.70 (dt, J=25.1, 8.0 Hz, 7H), 7.59 (t, J=7.5 Hz, 1H), 7.52 (t, J=7.6 Hz, 1H), 7.31 (t, J=7.6 Hz, 1H), 6.71 (d, J=7.8 Hz, 1H), 6.31 (s, 1H).

¹³C{¹H}NMR (101 MHz, CDCl₃) δ 169.14, 140.01, 138.76, 137.98, 137.33, 133.07, 131.77, 131.05, 130.73, 130.69, 130.06, 129.19, 129.15, 128.92, 128.74, 128.19, 127.92, 126.83, 125.76, 125.72, 125.68, 125.65, 125.48, 122.78, 120.08, 78.38.

¹⁹F{¹H}NMR (376 MHz, CDCl₃) δ −62.57.

HRMS (DART-TOF) C₂₁H₁₃F₃O₂ [M+H]⁺ calcd: 355.09404 found: 355.09592.

### Synthesis and Characterization of 7-(4-(trifluoromethyl)phenyl)dibenzo[c,e]oxepine-5(7H)-thione (S20, p-CF₃PhDOT)

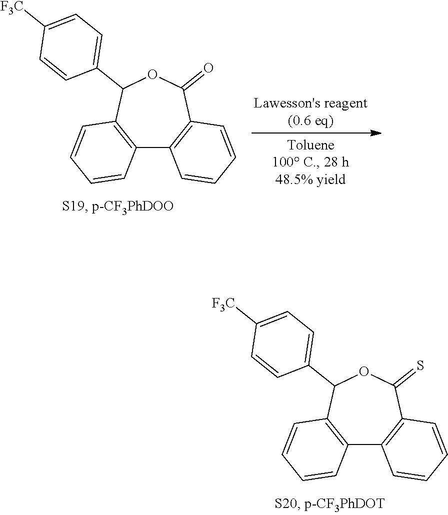 [SMILES: O=C1OC(c2ccc(C(F)(F)F)cc2)c2ccccc2-c2ccccc21 ; FC(F)(F)c1ccc(C2OC(=S)c3ccccc3-c3ccccc32)cc1]

To a flame-dried 250 mL round-bottom flask equipped with a magnetic stir bar were added 7-(4-(trifluoromethyl)phenyl)dibenzo[c,e]oxepin-5(7H)-one (S19, 3.00 g, 8.47 mmol, 1.0 equiv) and Lawesson's reagent (2.055 g, 5.08 mmol, 0.6 equiv). The flask was sealed with a rubber septum and purged with nitrogen. Anhydrous toluene (30 mL) was then added, and the resulting mixture was stirred in an oil bath preheated to 100° C. for 28 hours. The flask was removed from the oil bath and allowed to cool to room temperature. The reaction mixture was filtered through a pad of Celite, which was then washed with additional EtOAc to fully elute and collect the yellow-colored product. The combined filtrates were concentrated under reduced pressure using a rotary evaporator and then kept under high vacuum for several hours to ensure complete removal of the solvent (Note: Residual toluene can affect the purification). The crude residue was purified by column chromatography on silica gel, using a gradient from DCM:Hex=1:6 to 1:4. The product was isolated as a yellow solid (1.52 g, 48.5% yield).

¹H NMR (400 MHz, CDCl₃) δ 8.27-8.16 (m, 1H), 7.81-7.65 (m, 6H), 7.60 (d, J=7.7 Hz, 1H), 7.52 (td, J=7.2, 4.1 Hz, 2H), 7.33 (t, J=7.6 Hz, 1H), 6.74 (d, J=7.8 Hz, 1H), 6.40 (s, 1H).

¹³C{¹H}NMR (101 MHz, CDCl₃) δ 214.16, 139.21, 139.19, 138.73, 137.55, 134.45, 133.66, 132.33, 131.44, 131.12, 130.79, 130.47, 130.10, 128.84, 128.83, 128.67, 128.44, 128.03, 127.97, 126.44, 125.72, 125.69, 125.65, 125.61, 125.33, 122.62, 119.92, 83.16.

¹⁹F{¹H}NMR (376 MHz, CDCl₃) δ −62.57.

HRMS (DART-TOF) C₂₁H₁₃F₃OS [M+H]⁺ calcd: 371.07120 found: 371.07364.

### Synthesis and Characterization of 5′-fluoro-2′-vinyl-[1,1′-biphenyl]-2-carbaldehyde (S22)

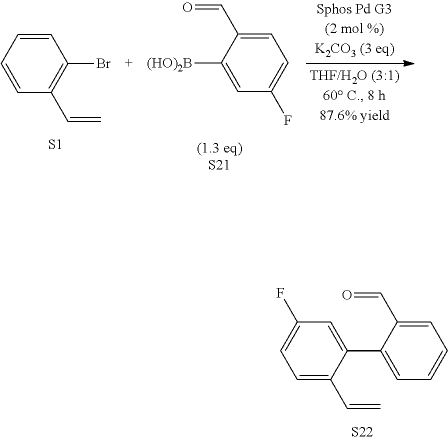

To an oven-dried 250 mL round-bottom flask were sequentially added Sphos Pd G3 (715 mg, 0.92 mmol, 0.02 equiv), 5-fluoro-2-formyl phenylboronic acid (10 g, 59.6 mmol, 1.3 equiv), and K₂CO₃ (19 g, 137.4 mmol, 3.0 equiv) inside a nitrogen-filled glovebox. The flask was then sealed with a rubber septum and removed from the glovebox. Separately, a mixture of THF (90 mL) and water (30 mL) was purged with argon for 40 minutes in a Schlenk flask to remove oxygen. Subsequently, 110 mL of this degassed solvent mixture was transferred into the reaction flask via syringe. 2-Bromostyrene (5.74 mL, 45.8 mmol, 1.0 equiv) was added to the flask against a nitrogen flow. The resulting mixture was stirred vigorously in an oil bath preheated to 60° C. for 8 hours. The flask was removed from the oil bath and allowed to cool to room temperature. The reaction mixture was then filtered through a silica pad, which was further washed with additional ethyl acetate to completely elute the product. The combined filtrates were dried over Na₂SO₄ and concentrated under reduced pressure using a rotary evaporator. The crude residue was purified by column chromatography on silica gel using EA:Hex=1:30 as the eluent. The product was obtained as a colorless liquid (9.08 g, 87.6% yield).

¹H NMR (400 MHz, CDCl₃) δ 9.62 (s, 1H), 8.06 (dd, J=8.7, 5.9 Hz, 1H), 7.66 (dd, J=7.9, 1.3 Hz, 1H), 7.42 (td, J=7.6, 1.4 Hz, 1H), 7.34 (td, J=7.5, 1.3 Hz, 1H), 7.22 (dd, J=7.6, 1.4 Hz, 1H), 7.18 (td, J=8.4, 2.6 Hz, 1H), 7.01 (dd, J=9.1, 2.6 Hz, 1H), 6.40 (dd, J=17.4, 11.0 Hz, 1H), 5.68 (dd, J=17.5, 1.0 Hz, 1H), 5.19 (dd, J=10.9, 1.0 Hz, 1H).

¹³C{¹H}NMR (101 MHz, CDCl₃) δ 190.25, 166.72, 164.16, 147.41, 147.32, 136.85, 135.08, 135.06, 134.29, 130.95, 130.92, 130.40, 130.12, 130.02, 128.98, 127.69, 125.52, 118.11, 117.90, 116.79, 115.67, 115.45, 30.31.

¹⁹F{¹H}NMR (376 MHz, CDCl₃) δ −103.41 (q, J=7.8 Hz, 1F).

HRMS (DART-TOF) C₁₅H₁₁FO [M+H]⁺ calcd: 227.08667 found: 227.08650.

### Synthesis and Characterization of 10-fluoro-7-(4-(trifluoromethyl)phenyl)dibenzo[c,e]oxepin-5(7H)-one (S24, F-p-CF₃PhDOO)

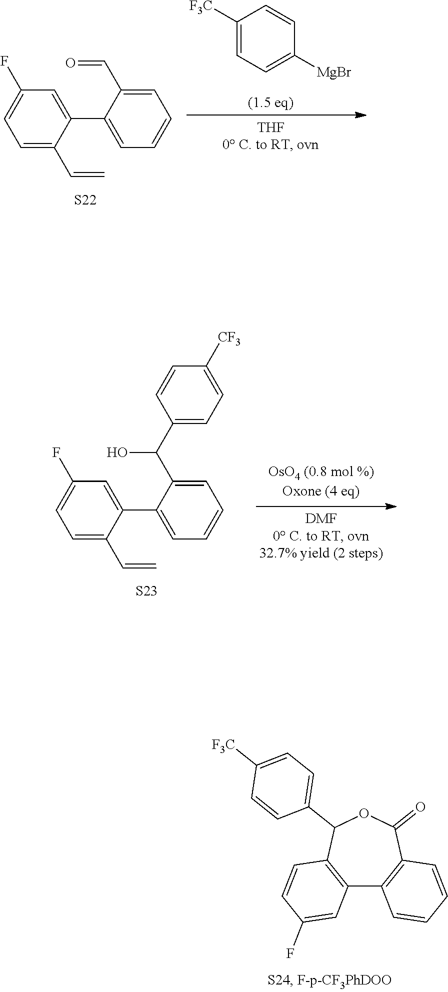

To a flame-dried 100 mL round-bottom flask, equipped with a magnetic stir bar, were added magnesium turnings (1.07 g, 44 mmol, 1.1 equiv) and LiCl (1.7 g, 40 mmol, 1.0 equiv) inside a nitrogen-filled glovebox. The flask was sealed with a rubber septum and then removed from the glovebox. Anhydrous THF (40 mL) was added to the flask via syringe, against a flow of nitrogen. After adding a small portion of 4-bromobenzotrifluoride (0.2 mL out of 5.6 mL total), the mixture was stirred at 50° C. until the magnesium was activated and the color of the solution changed to brown. The mixture was subsequently cooled to 0° C., after which the remaining 4-bromobenzotrifluoride (5.4 mL out of the initial 5.6 mL, 40 mmol, 1.0 equiv) was added dropwise. The mixture was stirred for an additional 30 minutes at 0° C., followed by 2 hours at room temperature. The resulting dark brown solution of 1 M 4-(trifluoromethyl) phenyl magnesium bromide in THF was used for the subsequent reaction.

A flame-dried 250 mL round-bottom flask equipped with a magnetic stir bar was sealed with a rubber septum and charged with nitrogen. Then, 5′-fluoro-2′-vinyl-[1,1′-biphenyl]-2-carbaldehyde (5.29 g, 23.4 mmol, 1.0 equiv) and anhydrous THF (35 mL) were added into the flask via syringes. The resulting solution was cooled to 0° C. in an ice bath under a nitrogen atmosphere. 1M 4-(trifluoromethyl)phenylmagnesium bromide solution in THF (35 mL, 35 mmol, 1.5 equiv) was added dropwise while stirring over a period of 30 minutes. The resulting mixture was allowed to stir at 0° C. for an additional 30 minutes, and then stirred at room temperature overnight. The reaction was quenched by the addition of water (10 mL), followed by a saturated aqueous solution of NH₄Cl (100 mL). The aqueous layer was extracted with EtOAc (3×70 mL). The combined organic layers were dried over Na₂SO₄ and concentrated under reduced pressure using a rotary evaporator. The crude residue was purified by column chromatography on silica gel using EA:Hex=1:10 as the eluent. All fractions containing the product were collected and the solvent was removed. The resulting colorless oil was used for the subsequent reaction.

The colorless oil was transferred into a 250 mL round-bottom flask equipped with a magnetic stir bar and kept under vacuum overnight. After the addition of Oxone (28.7 g, 93.5 mmol, 4.0 equiv), the flask was sealed with a rubber septum and charged with nitrogen. Anhydrous DMF (40 mL) was added via syringe against a nitrogen flow, and the resulting mixture was stirred vigorously until the colorless oil had fully homogenized with the solvent. The mixture was then cooled to 0° C. in an ice bath. A 2.5 wt % OsO₄ solution in t-BuOH (2.44 mL, 0.187 mmol, 0.008 equiv) was added dropwise to the flask at 0° C. The resulting black mixture was stirred at 0° C. for an additional 30 minutes and then allowed to stir overnight at room temperature. After the addition of water (40 mL), the mixture was transferred to a separatory funnel, brine (100 mL) was added, and the aqueous layer was extracted with EtOAc (3×100 mL). The combined organic layers were thoroughly washed with an aqueous solution of Na₂SO₃ (2×200 mL) to quench residual OsO₄ and then washed with water (2×200) and brine (2×200 mL) to remove residual DMF. The organic layer was dried over Na₂SO₄ and then concentrated under reduced pressure using a rotary evaporator. The crude residue was purified by column chromatography on silica gel, using a gradient from EA:Hex=1:9 to 1:8. The product was obtained as a white solid (2.845 g, 32.7% yield over 2 steps).

¹H NMR (400 MHz, CDCl₃) δ 8.04 (d, J=7.7 Hz, 1H), 7.76 (dd, J=14.5, 7.6 Hz, 3H), 7.70-7.57 (m, 4H), 7.38 (dd, J=9.3, 2.6 Hz, 1H), 6.99 (td, J=8.4, 2.6 Hz, 1H), 6.69 (dd, J=8.7, 5.5 Hz, 1H), 6.26 (s, 1H).

¹³C{¹H}NMR (101 MHz, CDCl₃) δ 168.77, 164.68, 162.20, 140.96, 140.87, 139.85, 136.16, 136.14, 134.10, 134.07, 133.24, 131.94, 131.52, 131.20, 130.87, 130.72, 130.55, 129.50, 129.09, 129.03, 129.01, 128.12, 127.83, 125.86, 125.82, 125.79, 125.75, 125.42, 122.71, 120.00, 116.18, 115.95, 115.59, 115.37, 77.72.

¹⁹F{¹H}NMR (376 MHz, CDCl₃) δ −62.61 (s, 3F), −110.82 (q, J=8.3 Hz, 1F).

HRMS (DART-TOF) C₂₁H₁₂F₄O₂ [M+H]⁺ calcd: 373.08462 found: 373.08487.

### Synthesis and Characterization of 10-fluoro-7-(4-(trifluoromethyl)phenyl)dibenzo[c,e]oxepine-5(7H)-thione (S25, F-p-CF₃PhDOT)

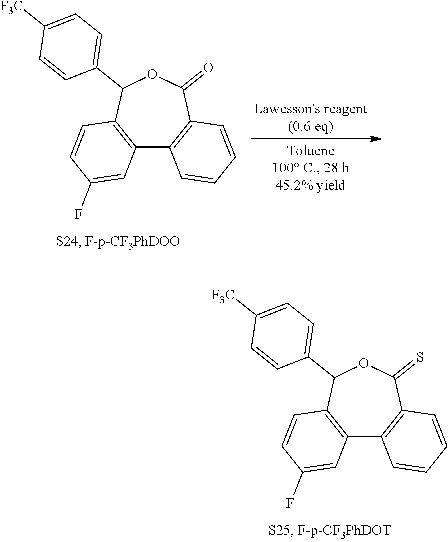 [SMILES: O=C1OC(c2ccc(C(F)(F)F)cc2)c2ccc(F)cc2-c2ccccc21 ; Fc1ccc2c(c1)-c1ccccc1C(=S)OC2c1ccc(C(F)(F)F)cc1]

To a flame-dried 100 mL round-bottom flask equipped with a magnetic stir bar were added 10-fluoro-7-(4-(trifluoromethyl)phenyl)dibenzo[c,e]oxepin-5(7H)-one (S24, 2.845 g, 7.64 mmol, 1.0 equiv) and Lawesson's reagent (1.854 g, 4.59 mmol, 0.6 equiv). The flask was sealed with a rubber septum and charged with nitrogen. Anhydrous toluene (20 mL) was then added to the flask, and the resulting mixture was stirred in an oil bath preheated to 100° C. for 28 hours. After this period, the flask was removed from the oil bath and allowed to cool to room temperature. It was then further cooled in a refrigerator to approximately 2° C. The reaction mixture was filtered through a silica pad, which was subsequently washed with additional EtOAc to completely elute and collect the yellow-colored product. The combined filtrate was concentrated under reduced pressure using a rotary evaporator and then kept under high vacuum for several hours to ensure complete removal of solvent (Note: Residual toluene can affect the purification process). The crude residue was purified by column chromatography on silica gel, using a gradient from DCM:Hex=1:6 to 1:4. The product was isolated as a yellow solid (1.34 g, 45.2% yield).

¹H NMR (400 MHz, CDCl₃) δ 8.21 (dd, J=7.8, 1.3 Hz, 1H), 7.82-7.66 (m, 5H), 7.61-7.49 (m, 2H), 7.39 (dd, J=9.2, 2.6 Hz, 1H), 7.01 (td, J=8.4, 2.7 Hz, 1H), 6.72 (dd, J=8.7, 5.5 Hz, 1H), 6.34 (s, 1H).

¹³C{¹H}NMR (101 MHz, CDCl₃) δ 213.57, 164.64, 162.16, 140.95, 140.86, 139.22, 139.06, 133.78, 133.68, 133.65, 133.25, 133.23, 132.43, 131.60, 131.28, 130.95, 130.63, 129.00, 128.73, 128.67, 128.65, 127.96, 127.87, 125.83, 125.79, 125.75, 125.72, 125.26, 122.55, 119.85, 115.82, 115.59, 115.57, 115.35, 82.43.

¹⁹F{¹H}NMR (376 MHz, CDCl₃) δ −62.63 (s, 3F), −110.46 (td, J=8.6, 5.3 Hz, 1F).

HRMS (DART-TOF) C₂₁H₁₂F₄OS [M+H]⁺ calcd: 389.06177 found: 389.06178.

### Synthesis and Characterization of 7-(perfluorophenyl)dibenzo[c,e]oxepin-5(7H)-one (S27, PFPhDOO)

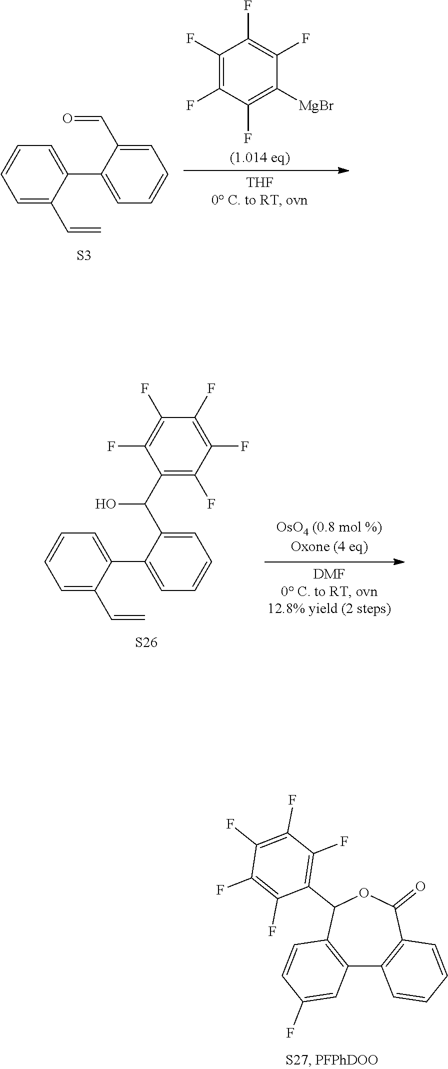

To a flame-dried 250 mL round-bottom flask equipped with a magnetic stir bar was added 2′-vinyl-[1,1′-biphenyl]-2-carbaldehyde (S3, 10.27 g, 49.3 mmol, 1.0 equiv). The flask was sealed with a rubber septum, then evacuated and backfilled with nitrogen four times. Anhydrous THF (60 mL) was added, and the mixture was stirred at room temperature until S3 had fully dissolved. The mixture was then cooled to 0° C. in an ice bath. 0.5 M pentafluorophenyl magnesium bromide solution in Et₂O (100 mL, 50 mmol, 1.014 equiv) was added dropwise while stirring over a period of 30 minutes. The resulting mixture was allowed to stir at 0° C. for an additional 20 minutes, and then stirred at room temperature overnight. The reaction was quenched by the addition of water (10 mL), followed by a saturated aqueous solution of NH₄Cl (150 mL). The aqueous layer was extracted with EtOAc (3×100 mL). The combined organic layers were dried over Na₂SO₄ and concentrated under reduced pressure using a rotary evaporator. The crude residue was purified by column chromatography on silica gel, using a gradient from EA:Hex=1:10 to 1:8. All fractions containing the product were collected and the solvent was removed. The resulting viscous, colorless oil was used for the subsequent reaction.

The viscous, colorless oil was transferred into a 250 mL round-bottom flask equipped with a magnetic stir bar and kept under vacuum overnight. After the addition of Oxone (60.6 g, 197.2 mmol, 4.0 equiv), the flask was sealed with a rubber septum and charged with nitrogen. Anhydrous DMF (80 mL) was added via syringe against a nitrogen flow, and the resulting mixture was stirred vigorously until the colorless oil had fully homogenized with the solvent. The mixture was then cooled to 0° C. in an ice bath. A 2.5 wt % OsO₄ solution in t-BuOH (5.0 mL, 0.394 mmol, 0.008 equiv) was added dropwise to the flask at 0° C. The resulting mixture was stirred at 0° C. for an additional 30 minutes and then allowed to stir overnight at room temperature. After the addition of water (50 mL), the mixture was transferred to a separatory funnel, brine (100 mL) was added, and the aqueous layer was extracted with EtOAc (3×100 mL). The combined organic layers were thoroughly washed with an aqueous solution of Na₂SO₃ (2×200 mL) to quench residual OsO₄ and then washed with water (2×200) and brine (2×200 mL) to remove residual DMF. The organic layer was dried over Na₂SO₄ and then concentrated under reduced pressure using a rotary evaporator. The purification of the crude residue required two sequential column chromatographies on silica gel. After the first chromatography using EA:Hex=1:8 as the eluent, all fractions containing the product were collected and concentrated under reduced pressure using a rotary evaporator. The residue was then subjected to a second column chromatography, with a gradient from DCM:Hex=1:5 to 1:3. The product was isolated as a white solid (2.373 g, 12.8% yield over 2 steps).

¹H NMR (400 MHz, CDCl₃) δ 8.06 (dd, J=7.9, 1.4 Hz, 1H), 7.81-7.65 (m, 3H), 7.60 (qd, J=7.7, 1.2 Hz, 2H), 7.42 (td, J=7.6, 1.3 Hz, 1H), 7.04 (dd, J=7.4, 2.1 Hz, 1H), 6.54 (s, 1H).

¹³C{¹H}NMR (101 MHz, CDCl₃) δ 168.54, 146.46, 143.96, 143.15, 140.59, 139.20, 138.52, 136.98, 136.68, 133.75, 133.31, 132.11, 130.63, 129.65, 129.50, 129.46, 129.04, 128.80, 125.61, 109.94, 71.30.

¹⁹F{¹H}NMR (376 MHz, CDCl₃) δ −151.42 (t, J=22.7 Hz, 2F), −160.44 (t, J=27.6 Hz, 3F).

Note: The ¹³C NMR peaks at δ 146.46, 143.96, 143.15, 140.59, 139.20, 136.68, and 109.94 appear as multiplets due to the coupling with fluorine.

HRMS (DART-TOF) C₂₀H₉F₅O₂[M+H]⁺ calcd: 377.05955 found: 377.06006.

### Synthesis and Characterization of 7-(perfluorophenyl)dibenzo[c,e]oxepine-5(7H)-thione (S28, PFPhDOT)

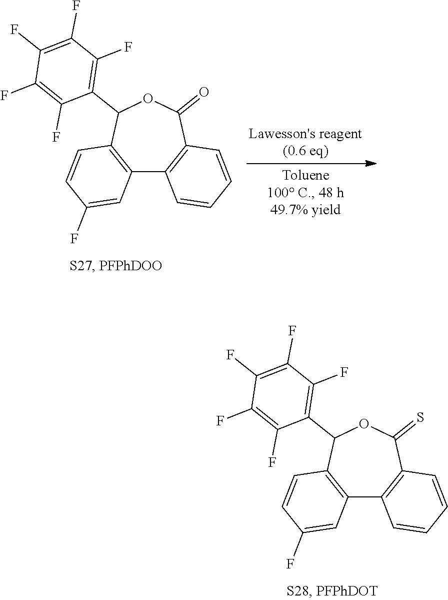 [SMILES: O=C1OC(c2c(F)c(F)c(F)c(F)c2F)c2ccc(F)cc2-c2ccccc21 ; Fc1ccc2c(c1)-c1ccccc1C(=S)OC2c1c(F)c(F)c(F)c(F)c1F]

To a flame-dried 250 mL round-bottom flask, equipped with a magnetic stir bar, were added 7-(perfluorophenyl)dibenzo[c,e]oxepin-5(7H)-one (S27, 2.373 g, 6.31 mmol 1.0 equiv) and Lawesson's reagent (1.53 g, 3.78 mmol, 0.6 equiv). The flask was sealed with a rubber septum and charged with nitrogen. Anhydrous toluene (25 mL) was then added, and the resulting mixture was stirred in an oil bath preheated to 100° C. for 48 hours. The flask was removed from the oil bath and allowed to cool to room temperature before being further cooled in a refrigerator to approximately 2° C. The reaction mixture was then filtered through a pad of silica, which was subsequently washed with additional EtOAc to completely elute and collect the yellow-orange product. The combined filtrate was concentrated under reduced pressure using a rotary evaporator and was kept under high vacuum for several hours to ensure complete removal of solvent (Note: Residual toluene can affect the purification process). The crude residue was purified by column chromatography on silica gel, using a gradient from DCM:Hex=1:6 to 2:5. The product was isolated as a foamy yellow-orange solid (1.23 g, 49.7% yield).

¹H NMR (400 MHz, CDCl₃) δ 8.24 (dd, J=8.0, 1.4 Hz, 1H), 7.77-7.65 (m, 2H), 7.64-7.49 (m, 3H), 7.44 (td, J=7.7, 1.3 Hz, 1H), 7.06 (d, J=7.8 Hz, 1H), 6.62 (s, 1H).

¹³C{¹H}NMR (101 MHz, CDCl₃) δ 213.44, 146.58, 144.06, 143.35, 140.79, 139.26, 138.70, 138.34, 136.76, 134.20, 134.15, 133.54, 132.64, 130.77, 129.38, 129.27, 128.83, 128.68, 125.44, 109.48, 75.70.

¹⁹F{¹H}NMR (376 MHz, CDCl₃) δ −150.99 (t, J=22.4 Hz), −160.09 (d, J=29.2 Hz).

Note: The ¹³C NMR peaks at δ 146.58, 144.06, 143.35, 140.79, 139.26, 136.76, and 109.48 appear as multiplets due to the coupling with fluorine.

HRMS (DART-TOF) C₂₀H₉F₅OS [M+H]⁺ calcd: 393.03670 found: 393.03794.

Discussion about the Side Products in Synthesis

The side products formed from the thionation of PhDOO to synthesize PhDOT were characterized as the isomerized thiolactone 7-phenyldibenzo[c,e]thiepin-5(7H)-one (PhDTO) and the overthionated 7-phenyldibenzo[c,e]thiepine-5(7H)-thione (PhDTT). The HRMS results matched well with the assignments.

The quantities of the four major species, PhDOO, PhDOT, PhDTT, and PhDTO, in the reaction mixture were assessed using a small-scale test reaction (200 μmol). After the reaction, the mixture was filtered through a pad of celite, the solvent was then evaporated, and the residue was analyzed by NMR, using either 200 μmol of 1,1,2,2-tetrachloroethane (21 μL) or dibromomethane (14 μL) as an internal standard.

Following 4 hours of thionation, the crude mixture comprised 52 μmol of PhDOO, 109 μmol of PhDOT, 14 μmol of PhDTT, and 8 μmol of PhDTO, indicating a 74% conversion and a 55% crude yield. The stability of the species on silica was assessed by applying the crude mixture onto a silica pad, eluting it after a 3-hour period, and subsequently reanalyzing it using NMR. No notable change in integrations was observed, suggesting that the products neither decompose nor interconvert on silica.

Prolonging the reaction time to 10 hours did not appreciably increase the yield; the mixture formed 115 μmol of PhDOT (57% crude yield), while further consumption of the starting material PhDOO (87% conversion) led to increased production of the side products PhDTT and PhDTO.

Scaling up this reaction resulted in a decrease in the yield of PhDOT synthesis. This reduction is presumably due to the decreased efficiency of the reaction that converts PhDOO to PhDOT, while the efficiency of the isomerization process from PhDOT to PhDTO remained unchanged.

### Synthesis and Characterization of Polymers

### Evaluation of the Reactivity of MeDOT, Me₂DOT, and PhDOT

Stock solutions of ACHN (1.22 mg/1000 μL, 5 mM), 2-cyano-2-propyl benzodithioate (11.1 mg/1000 μL, 50 mM), and 1,4-bis(trimethylsilyl)benzene (55.5 mg/1000 μL, 250 mM) in Tol-d8 were prepared under ambient atmosphere and used for the following reactions.

### Evaluation of the Reactivity of MeDOT

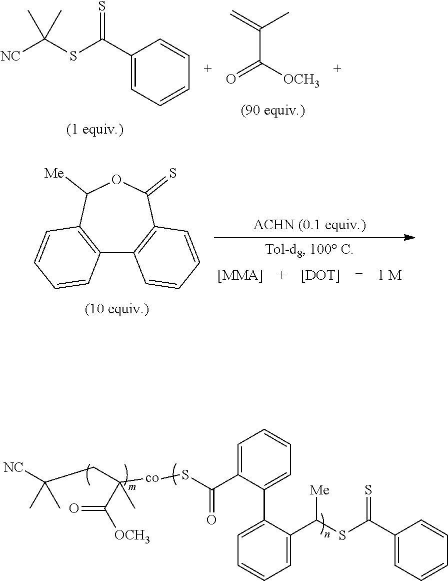

To a 4 mL scintillation vial, 12.0 mg of MeDOT (50 μmol, 10 equiv.), 153 μL of Tol-d8, 100 μL of 1,4-bis(trimethylsilyl)benzene stock (25 μmol, 5.0 equiv.), 100 μL of 2-cyano-2-propyl benzodithioate stock (5 μmol, 1.0 equiv.), and 100 μL of ACHN stock (0.5 μmol, 0.1 equiv.) solutions were added sequentially. Following the addition of 47.9 μL MMA (450 μmol, 90 equiv.), the resulting mixture was vortexed and then transferred into a J-Young NMR tube. The solution was degassed by four careful freeze-pump-thaw cycles. The tube was then placed in an oil bath pre-heated to 100° C. At each time point, the tube was removed from the oil bath, rapidly cooled to room temperature in a water bath, and subsequently subjected to NMR analysis to measure the conversion of the monomers. No consumption of MeDOT was observed, while MMA polymerized to form PMMA (FIG. 31A).

### Evaluation of the Reactivity of Me₂DOT

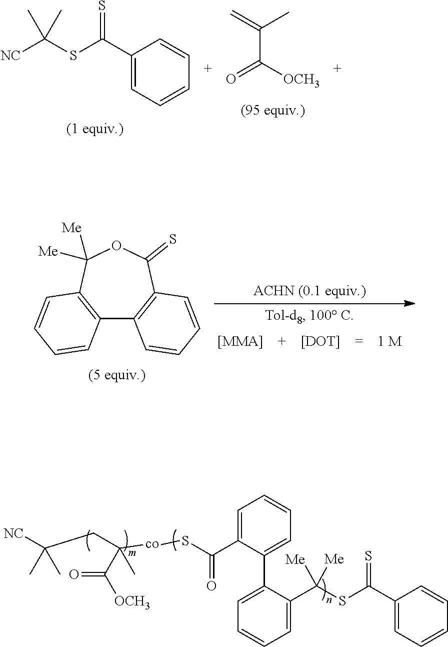

To a 4 mL scintillation vial, 6.4 mg of Me₂DOT (25 μmol, 5.0 equiv.), 153 μL of Tol-d8, 100 μL of 1,4-bis(trimethylsilyl)benzene stock (25 μmol, 5.0 equiv.), 100 μL of 2-cyano-2-propyl benzodithioate stock (5 μmol, 1.0 equiv.), and 100 μL of ACHN stock (0.5 μmol, 0.1 equiv.) solutions were added sequentially. Following the addition of 50.6 μL MMA (475 μmol, 95 equiv.), the resulting mixture was vortexed and then transferred into a J-Young NMR tube. The solution was degassed by four careful freeze-pump-thaw cycles. The tube was then placed in an oil bath pre-heated to 100° C. At each time point, the tube was removed from the oil bath, rapidly cooled to room temperature in a water bath, and subsequently subjected to NMR analysis to measure the conversion of the monomers. Me₂DOT gradually converted into an unknown species but did not copolymerize with MMA The polymerization of MMA was significantly retarded in the presence of Me₂DOT (FIG. 31B).

### Evaluation of the Reactivity of PhDOT

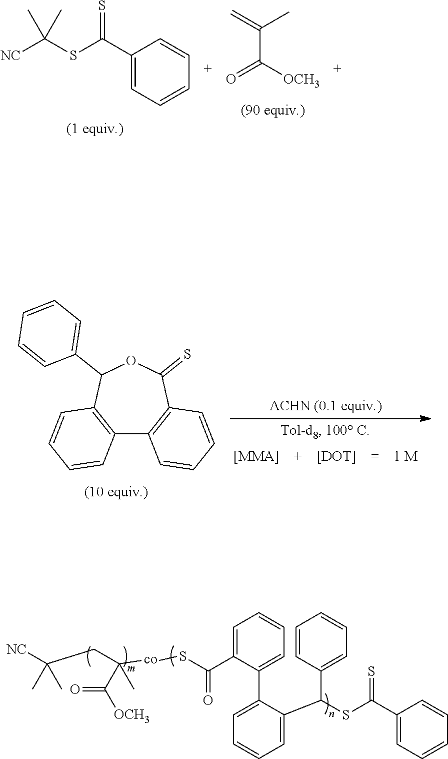

To a 4 mL scintillation vial, 15.1 mg of PhDOT (50 μmol, 10 equiv.), 153 μL of Tol-d8, 100 μL of 1,4-bis(trimethylsilyl)benzene stock (25 μmol, 5.0 equiv.), 100 μL of 2-cyano-2-propyl benzodithioate stock (5 μmol, 1.0 equiv.), and 100 μL of ACHN stock (0.5 μmol, 0.1 equiv.) solutions were added sequentially. Following the addition of 47.9 μL MMA (450 μmol, 90 equiv.), the resulting mixture was vortexed and then transferred into a J-Young NMR tube. The solution was degassed by four careful freeze-pump-thaw cycles. The tube was then placed in an oil bath pre-heated to 100° C. At each time point, the tube was removed from the oil bath, rapidly cooled to room temperature in a water bath, and subsequently subjected to NMR analysis to measure the conversion of the monomers. PhDOT was consumed throughout its copolymerization with MMA (FIG. 32).

### Synthesis of P(MMA-Co-bDOT)

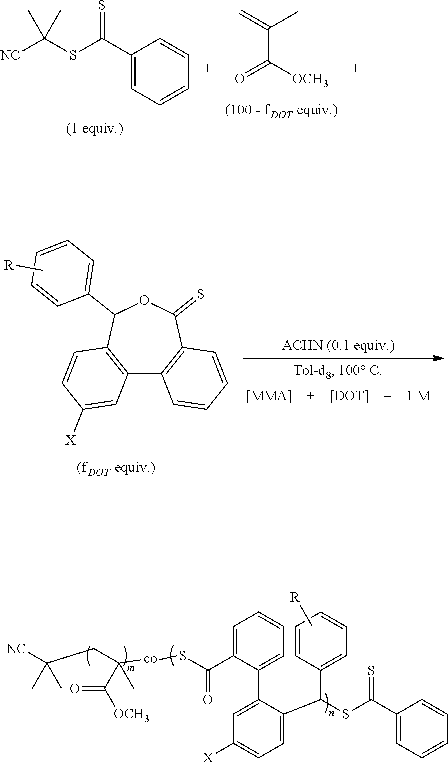

Stock solutions of ACHN (1.22 mg/1000 μL, 5 mM), 2-cyano-2-propyl benzodithioate (11.1 mg/1000 μL, 50 mM), and 1,4-bis(trimethylsilyl)benzene (55.5 mg/1000 μL, 250 mM) in Tol-d8 were prepared in a glovebox and used for the following reactions for consistency.

To a 4 mL scintillation vial, 15.1 mg of PhDOT (50 μmol, 10 equiv.), 153 μL of Tol-d8, 100 μL of 1,4-bis(trimethylsilyl)benzene stock (25 μmol, 5.0 equiv.), 100 μL of 2-cyano-2-propyl benzodithioate stock (5 μmol, 1.0 equiv.), and 100 μL of ACHN stock (0.5 μmol, 0.1 equiv.) solutions were added sequentially. Following the addition of 47.9 μL MMA (450 μmol, 90 equiv.), the resulting mixture was vortexed and then transferred into an NMR tube. The tube was securely sealed with a cap, reinforced with electrical tape, and then removed from the glovebox. Subsequently, the NMR tube was submerged in an oil bath preheated to 100° C. After heating for 60 hours at 100° C., the tube was removed from the oil bath and allowed to cool to room temperature. The reaction was quenched by exposure to air. ¹H NMR analysis was conducted before and after the reaction to measure the conversion of monomers. The solvent was removed with the aid of a rotary evaporator, and the residue was subjected to preparative SEC to remove small molecules. The resulting polymer was analyzed by analytical SEC and NMR (FIGS. 33-36, Table 2).

The analogous experiments were done for aryl bDOTs, p-MePhDOT (63.3 mg, 200 μmol, 10 equiv.), p-CF₃PhDOT (74.1 mg, 200 μmol, 10 equiv.), F-p-CF₃PhDOT (77.7 mg, 200 μmol, 10 equiv.), and PFPhDOT (78.4 mg, 200 μmol, 10 equiv.).

Deconstruction of P(MMA-Co-bDOT) into O(MMA-Co-bDOT)

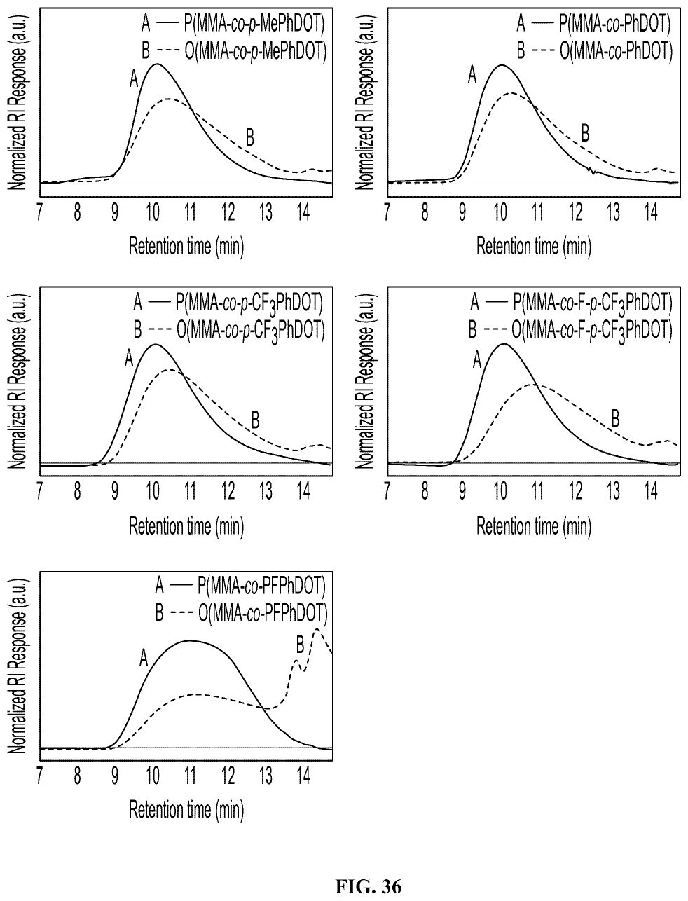

To a 4 mL scintillation vial containing P(MMA-co-bDOT) was added a magnetic stir bar. The vial was added 950 μL propylamine and 50 μL 1,8-Diazabicyclo[5.4.0]undec-7-ene (DBU). The vial was capped with a screw-thread cap fitted with Teflon septa. The reaction mixture was stirred in an aluminum heating block preheated to 50° C. After stirring for 24 hours at 50° C., the vial was removed from the heating block and allowed to cool to room temperature. The reaction mixture was diluted with EtOAc (30 mL) and washed with 1 M aqueous solution of HCl (4×30 mL). The organic layer was dried over Na₂SO₄, and the solvent was removed with the aid of a rotary evaporator. The resulting O(MMA-co-bDOT) was analyzed by analytical SEC (FIG. 36 and Table 2).

TABLE 2
Summary of the molecular weight and dispersity of the polymers

| | | | |
|---|---|---|---|
| Name | Mₙ | M_{W} | Ð |

Before degradation

| | | | |
|---|---|---|---|
| P(MMA-co-PhDOT) | 5108 | 7543 | 1.477 |
| P(MMA-co-p-MePhDOT) | 5312 | 8084 | 1.522 |
| P(MMA-co-p-CF₃PhDOT) | 4802 | 7462 | 1.554 |
| P(MMA-co-F-p-CF₃PhDOT) | 4442 | 7124 | 1.604 |
| P(MMA-co-PFPhDOT) | 2857 | 8279 | 1.812 |

After degradation

| | | | |
|---|---|---|---|
| O(MMA-co-PhDOT) | 2590 | 6438 | 2.486 |
| O(MMA-co-p-MePhDOT) | 2430 | 6026 | 2.480 |
| O(MMA-co-p-CF₃PhDOT) | 2078 | 5707 | 2.746 |
| O(MMA-co-F-p-CF₃PhDOT) | 1735 | 4440 | 2.558 |
| O(MMA-co-PFPhDOT) | 742 | 2534 | 3.414 |

Synthesis of vPMMA and dPMMA(f_{bDOT})

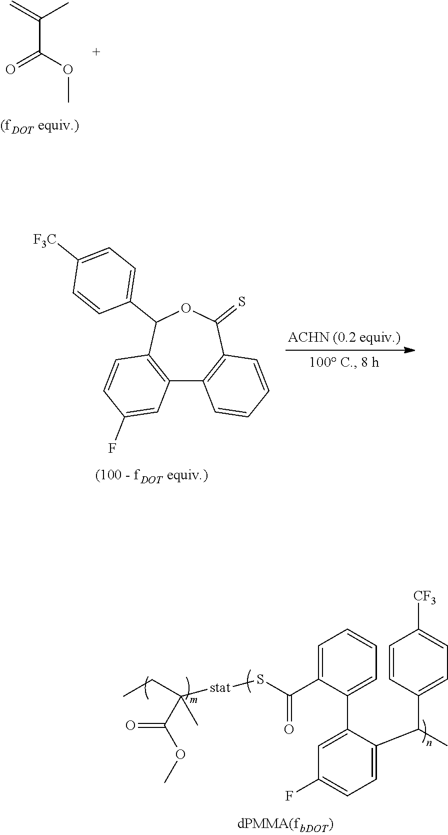

A stock solution of ACHN in MMA (53.6 mg in 7020 μL, 31.3 mM) was prepared in a glovebox and used for the following polymerizations to ensure consistency in the amount of the initiator.

Synthesis of vPMMA

200 μL of MMA and 300 μL of ACHN stock solution were added to each of the five 4 mL scintillation vials. This amounts to 500 μL of MMA (4.69 mmol, 100.0 equiv.) and 2.29 mg of ACHN (9.39 μmol, 0.2 equiv.) per vial. The vials were securely sealed with screw-thread caps fitted with Teflon septa and reinforced using electrical tape, and then removed from the glovebox. Subsequently, the vials were placed in an aluminum heating block preheated to 100° C. and maintained at this temperature for 8 hours.

The vials were removed from the heating block and allowed to cool to room temperature. The reaction mixture in one of the vials was diluted with CDCl₃ to obtain a total solution volume of 10 mL. From this, 500 μL was transferred to an NMR tube and subjected to quantitative ¹H NMR analysis to measure the amount of unreacted starting materials. 1,1,2,2-Tetrachloroethane was added as an internal standard for quantification. It was determined that 99.6% of the MMA had reacted.

The reaction mixtures from the five vials were dissolved in chloroform and combined to form a total solution volume of 40 mL. This solution was then added dropwise over 5 minutes into vigorously stirred MeOH (500 mL) to precipitate the polymer. The precipitated polymer was collected and purified through three successive precipitations in MeOH. The resulting white solid was pulverized and dried under high vacuum for approximately 24 hours until no further mass loss was observed, yielding vPMMA (2.004 g, 85.3%) as a white powder.

Synthesis of dPMMA(2.5)

45.6 mg of F-p-CF₃PhDOT (117.4 μmol, 2.5 equiv.), 187.5 μL of MMA, and 300 μL of ACHN stock solution were added to each of the five 4 mL scintillation vials. This resulted in each vial containing 45.6 mg of F-p-CF₃PhDOT (117.4 μmol, 2.5 equiv.), 487.5 μL of MMA (4.58 mmol, 97.5 equiv.) and 2.29 mg of ACHN (9.39 μmol, 0.2 equiv.). The vials were securely sealed with screw-thread caps fitted with Teflon septa and reinforced using electrical tape, and then removed from the glovebox. Subsequently, the vials were placed in an aluminum heating block preheated to 100° C. and maintained at this temperature for 8 hours.

The vials were removed from the heating block and allowed to cool to room temperature. The reaction mixture in one of the vials was diluted with CDCl₃ to obtain a total solution volume of 10 mL. From this, 500 μL was transferred to an NMR tube and subjected to quantitative ¹H NMR analysis to measure the amount of unreacted starting materials. 1,1,2,2-Tetrachloroethane was added as an internal standard for quantification. It was determined that 99.6% of the MMA and 98.6% of F-p-CF₃PhDOT has reacted.

The reaction mixtures from the five vials were dissolved in chloroform and combined to form a total solution volume of 40 mL. This solution was then added dropwise over 5 minutes into vigorously stirred MeOH (500 mL) to precipitate the polymer. The precipitated polymer was collected and purified through three successive precipitations in MeOH. The resulting white solid was pulverized and dried under high vacuum for approximately 24 hours until no further mass loss was observed, yielding dPMMA(2.5) (2.138 g, 84.9%) as a white powder.

Synthesis of dPMMA(5.0)

91.2 mg of F-p-CF₃PhDOT (234.7 μmol, 5.0 equiv.), 175 μL of MMA, and 300 μL of ACHN stock solution were added to each of the five 4 mL scintillation vials. This resulted in each vial containing 91.2 mg of F-p-CF₃PhDOT (234.7 μmol, 5.0 equiv.), 475 μL of MMA (4.46 mmol, 95.0 equiv.) and 2.29 mg of ACHN (9.39 μmol, 0.2 equiv.). The vials were securely sealed with screw-thread caps fitted with Teflon septa and reinforced using electrical tape, and then removed from the glovebox. Subsequently, the vials were placed in an aluminum heating block preheated to 100° C. and maintained at this temperature for 8 hours.

The vials were removed from the heating block and allowed to cool to room temperature. The reaction mixture in one of the vials was diluted with CDCl₃ to obtain a total solution volume of 10 mL. From this, 500 μL was transferred to an NMR tube and subjected to quantitative ¹H NMR analysis to measure the amount of unreacted starting materials. 1,1,2,2-Tetrachloroethane was added as an internal standard for quantification. It was determined that 99.5% of the MMA and 98.6% of F-p-CF₃PhDOT has reacted.

The reaction mixtures from the five vials were dissolved in chloroform and combined to form a total solution volume of 40 mL. This solution was then added dropwise over 5 minutes into vigorously stirred MeOH (500 mL) to precipitate the polymer. The precipitated polymer was collected and purified through three successive precipitations in MeOH. The resulting white solid was pulverized and dried under high vacuum for approximately 24 hours until no further mass loss was observed, yielding dPMMA(5.0) (2.091 g, 77.8%) as a white powder.

Synthesis of dPMMA(10)

182.3 mg of F-p-CF₃PhDOT (469.4 μmol, 10.0 equiv.), 150 μL of MMA, and 300 μL of ACHN stock solution were added to a 4 mL scintillation vial. This amounts to 182.3 mg of F-p-CF₃PhDOT (469.4 μmol, 10.0 equiv.), 450 μL of MMA (4.22 mmol, 90.0 equiv.) and 2.29 mg of ACHN (9.39 μmol, 0.2 equiv.). The vial was securely sealed with a screw-thread cap fitted with Teflon septa and reinforced using electrical tape, and then removed from the glovebox. Subsequently, the vial was placed in an aluminum heating block preheated to 100° C. and maintained at this temperature for 8 hours.

The vial was removed from the heating block and allowed to cool to room temperature. The reaction mixture was diluted with CDCl₃ to obtain a total solution volume of 10 mL. From this, 500 μL was transferred to an NMR tube and subjected to quantitative ¹H NMR analysis to measure the amount of unreacted starting materials. 1,1,2,2-Tetrachloroethane was added as an internal standard for quantification. It was determined that 99.2% of the MMA and 96.1% of F-p-CF₃PhDOT has reacted.

The solution was then added dropwise over 5 minutes into vigorously stirred MeOH (100 mL) to precipitate the polymer. The precipitated polymer was collected and purified through three successive precipitations in MeOH. The resulting white solid was pulverized and dried under high vacuum for approximately 24 hours until no further mass loss was observed, yielding dPMMA(10) (0.438 g, 72.4%) as a white powder.

Deconstruction of dPMMA(f_{bDoT}) into OMMA(f_{bDoT})

### General Deconstruction Procedure

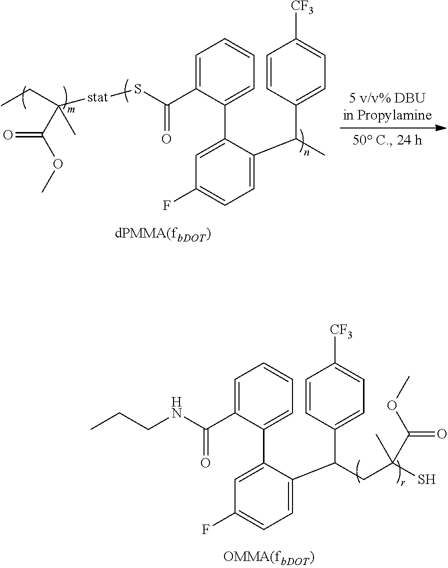

30 mg dPMMA(f_{bDOT}) was weighed into a 4 mL scintillation vial equipped with a magnetic stir bar. The vial was added 950 μL propylamine and 50 μL DBU. The vial was capped with a screw-thread cap fitted with Teflon septa. The reaction mixture was stirred in an aluminum heating block preheated to 50° C. After stirring for 24 hours at 50° C., the vial was removed from the heating block and allowed to cool to room temperature. The reaction mixture was diluted with EtOAc (30 mL) and washed with 1 M aqueous solution of HCl (4×20 mL). The organic layer was dried over Na₂SO₄, and the solvent was removed with the aid of a rotary evaporator.

### Control Deconstruction Experiment

30 mg of vPMMA was subjected to the general deconstruction procedure. The polymer obtained after the treatments was analyzed by analytical SEC. The SEC trace showed no difference compared to that of vPMMA (FIG. 37).

### Deconstruction Condition Screening

30 mg dPMMA(2.5) was subjected to the following four different deconstruction conditions.
1) 100% Propylamine (1000 μL propylamine), RT, 24 hours
2) 100% Propylamine (1000 μL propylamine), 50° C., 24 hours
3) 95% Propylamine+5% DBU (950 μL propylamine+50 μL DBU), RT, 24 hours
4) 95% Propylamine+5% DBU (950 μL propylamine+50 μL DBU), 50° C., 24 hours

The resulting reaction mixtures were worked-up following the general procedure. The polymer obtained after the treatments was analyzed by analytical SEC. The SEC trace showed no difference compared to that of OMMA(2.5), indicating that the addition of DBU and heating are unnecessary (FIG. 38).

### ¹H NMR Studies

¹H NMR experiments were conducted to confirm the estimated molecular weight of OMMA(5.0). dPMMA(5.0) was deconstructed in neat p-methoxy benzylamine (chosen for its 0-CH₃ signal not overlapping with that of PMMA) at room temperature. The resulting crude OMMA(5.0) was dissolved in EtOAc (20 mL) and washed thoroughly with a 1N aqueous solution of HCl (20 mL×8 times) to remove excess p-methoxy benzylamine. ¹H NMR analysis of the resulting OMMA(5.0) suggested an average of 25.1 MMA per end group, which aligns well with F_{bDOT} of 3.8% in the absence of CC diads. The calculated Mₙ from ¹H NMR integration was 3.0 kDa (525.62+25.1×100.11=3038.4), which is smaller than the 5.4 kDa measured from SEC. This discrepancy may be due to the slight inaccuracy of SEC in the low molar mass region; nevertheless, this confirms that F-p-CF₃PhDOT allows for the deconstruction of PMMA into small molar mass fragments.

Discussion about Bulk Polymerization

### Reactivity Ratios in Bulk Polymerization

The distribution of bDOTs can differ between RAFT polymerization and bulk FRP. These differences likely arise due to the reactivity ratios changing depending on the chemical environment. Reactivity ratios were not measured for bulk FRP, as MMA polymerization is known to autoaccelerate under these conditions, resulting in changes in reaction temperature and, accordingly, the reactivity ratios over the course of the polymerization.

The monomer conversions versus time for bulk free-radical copolymerization evidently shows autoacceleration. The fact that bDOT conversion closely parallels MMA conversion indicates, albeit not conclusively, that bDOT is being inserted into PMMA with a favorable distribution. The distribution of bDOT can change to some extent depending on the reaction scale and efficiency of heat transfer.

Comparison of bDOTs in Bulk Polymerization

dPMMAs with F_{bDOT}=1.25% were synthesized using both PhDOT and F-p-CF₃PhDOT as cleavable comonomers and were tested their degradation. The selection of an F_{bDOT} of 1.25% was due to the limited solubility of PhDOT in MMA.

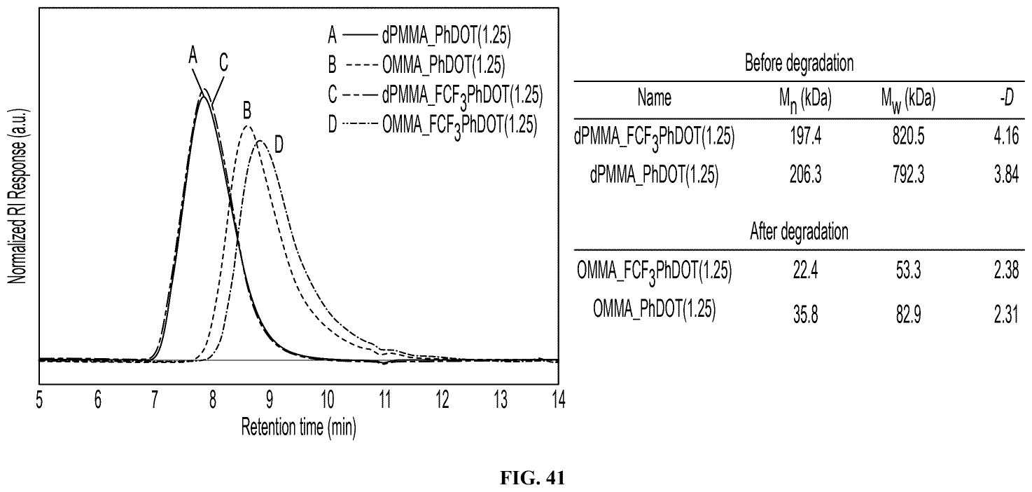

The SEC results revealed that the molar masses of the dPMMAs are almost indistinguishable, while the molar mass of the OMMA is >1.5 times larger when PhDOT is used as a cleavable comonomer (FIG. 41). This demonstrates that F-p-CF₃PhDOT is indeed a more efficient cleavable comonomer than PhDOT in bulk FRP.

Discussion about Initiators

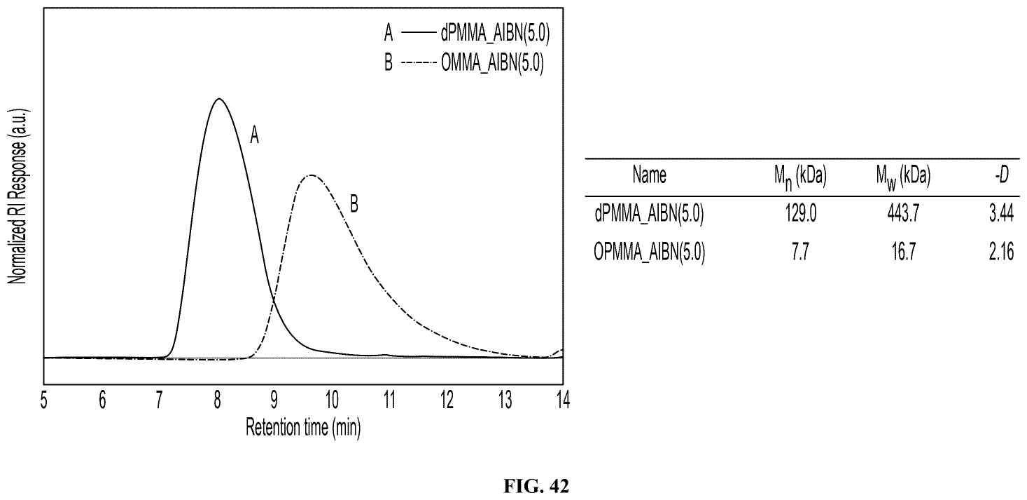

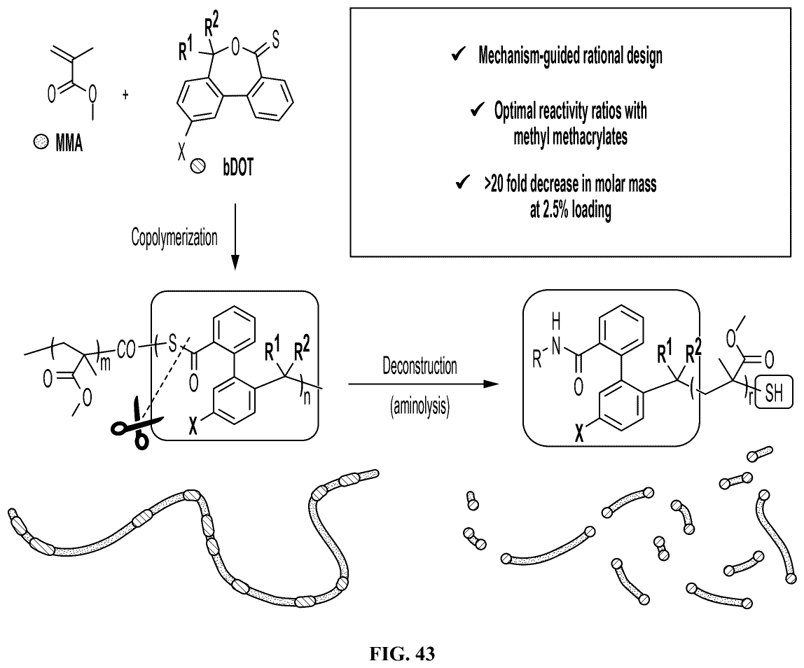

The use of AIBN as an initiator and conducting polymerization at a lower temperature (70° C.) did not alter the fact that the copolymer undergoes a significant (>16-fold) molar mass decrease upon deconstruction, as can be seen in FIG. 42.

### Determination of Reactivity Ratios

Kinetic Experiments for Aryl bDOTs

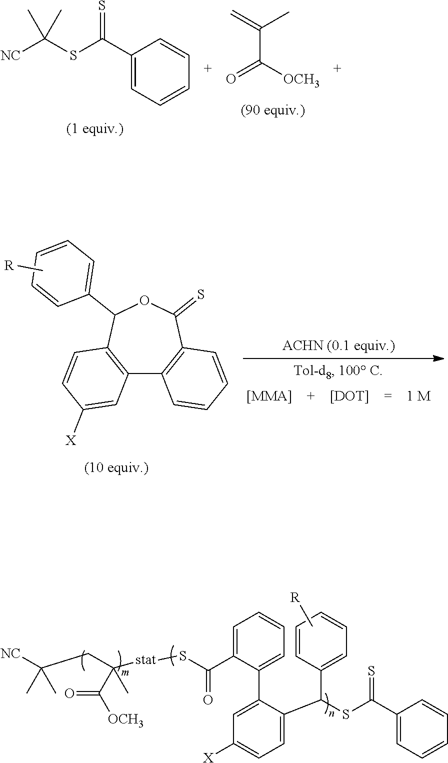

Stock solutions of ACHN (3.9 mg/3200 μL, 5 mM), 2-cyano-2-propyl benzodithioate (26.6 mg/2400 μL, 50 mM), and 1,4-bis(trimethylsilyl)benzene (133.2 mg/2400 μL, 250 mM) in Tol-d8 were prepared inside a nitrogen-filled glovebox.

60.5 mg of PhDOT (200 μmol, 10 equiv.), 400 μL of 1,4-bis(trimethylsilyl)benzene stock (100 μmol, 5.0 equiv.), 400 μL of 2-cyano-2-propyl benzodithioate stock (20 μmol, 1.0 equiv.), and 400 μL of ACHN stock (2 μmol, 0.1 equiv.) solutions were sequentially added to a 4 mL scintillation vial inside the glovebox. After the additions, 192 μL of MMA (1800 μmol, 90 equiv.) and approximately 600 μL of Tol-d8 were added to adjust the total volume of the mixture to 2 mL. The resulting orange solution was divided into 11 oven-dried NMR tubes (182 μL each). The tubes were securely sealed with caps, reinforced with electrical tape, and then removed from the glovebox. Subsequently, the NMR tubes were submerged in an oil bath preheated to 100° C., with vigorous stirring to maintain a homogeneous temperature. At each timepoint, ranging between 0 to 60 hours, one of the NMR tubes was removed from the oil bath, and the reaction was quenched by rapid cooling in an ice bath followed by exposure to air. The NMR spectra were evaluated after adding 300 μL of Tol-d8. The conversion of the monomers was measured using 1,4-bis(trimethylsilyl)benzene as the internal standard.

The analogous experiments were done for aryl bDOTs, p-MePhDOT (63.3 mg, 200 μmol, 10 equiv.), p-CF₃PhDOT (74.1 mg, 200 μmol, 10 equiv.), F-p-CF₃PhDOT (77.7 mg, 200 μmol, 10 equiv.), and PFPhDOT (78.4 mg, 200 μmol, 10 equiv.).

### Fitting Details

The Meyer-Lowry model was selected because of its known accuracy from low to high initial feed ratios, essential for measuring reactivity ratios in low CC feed.⁹² The experimental data were fitted to the following Meyer-Lowry equation:

$$conv = 1 - {( \frac{{f}_{A}}{{f}_{A}^{0}} )}^{\frac{{r}_{B}}{1 - {r}_{B}}} {( \frac{1 - {f}_{A}}{1 - {f}_{A}^{0}} )}^{\frac{{r}_{A}}{1 - {r}_{A}}} {( \frac{{f}_{A} ( 2 - {r}_{A} - {r}_{B} ) - {r}_{B} - 1}{{f}_{A}^{0} ( 2 - {r}_{A} - {r}_{B} ) - {r}_{B} - 1} )}^{\frac{( {r}_{A} {r}_{B} - 1 )}{( 1 - {r}_{A} ) ( 1 - {r}_{B} )}}$$

In the given equation, f_{A}⁰ represents the initial feed composition of A, f_{A} denotes the feed composition at a specific time point, conv is the conversion rate for the copolymerization at that time, and r_{A} and r_{B} are the reactivity ratios, with A and B corresponding to MMA and DOT, respectively.

An optimization algorithm, employing MATLAB's lsqnonlin function, was used to perform nonlinear least squares optimization to find the set of parameters that minimally deviate from the given experimental data, in terms of the sum of squared residuals.

### REFERENCES

(1) Lefay, C.; Guillaneuf, Y. Recyclable/degradable materials via the insertion of labile/cleavable bonds using a comonomer approach. Prog. Polym. Sci. 2023, 147, 101764.
(2) Shieh, P.; Hill, M. R.; Zhang, W.; Kristufek, S. L.; Johnson, J. A. Clip Chemistry: Diverse (Bio)(macro)molecular and Material Function through Breaking Covalent Bonds. Chem. Rev. 2021, 121 (12), 7059-7121.
(3) Shieh, P.; Zhang, W.; Husted, K. E. L.; Kristufek, S. L.; Xiong, B.; Lundberg, D. J.; Lem, J.; Veysset, D.; Sun, Y.; Nelson, K. A.; et al. Cleavable comonomers enable degradable, recyclable thermoset plastics. Nature 2020, 583 (7817), 542-547. DOI: 10.1038/s41586-020-2495-2.
(4) Gil, N.; Caron, B.; Siri, D.; Roche, J.; Hadiouch, S.; Khedaioui, D.; Ranque, S.; Cassagne, C.; Montarnal, D.; Gigmes, D.; et al. Degradable Polystyrene via the Cleavable Comonomer Approach. Macromolecules 2022, 55 (15), 6680-6694. DOI: 10.1021/acs.macromol.2c00651.
(5) Elliss, H.; Dawson, F.; Nisa, Q. u.; Bingham, N. M.; Roth, P. J.; Kopeć, M. Fully Degradable Polyacrylate Networks from Conventional Radical Polymerization Enabled by Thionolactone Addition. Macromolecules 2022, 55 (15), 6695-6702. DOI: 10.1021/acs.macromol.2c01140.
(6) Lloyd, E. M.; Cooper, J. C.; Shieh, P.; Ivanoff, D. G.; Parikh, N. A.; Mejia, E. B.; Husted, K. E. L.; Costa, L. C.; Sottos, N. R.; Johnson, J. A.; et al. Efficient Manufacture, Deconstruction, and Upcycling of High-Performance Thermosets and Composites. ACS Appl. Energy Mater. 2023, 1 (1), 477-485. DOI: 10.1021/acsaenm.2c00115.
(7) Shieh, P.; Nguyen, H. V. T.; Johnson, J. A. Tailored silyl ether monomers enable backbone-degradable polynorbornene-based linear, bottlebrush and star copolymers through ROMP. Nat. Chem. 2019, 11 (12), 1124-1132. DOI: 10.1038/s41557-019-0352-4.
(8) Chain Copolymerization. In Principles of Polymerization, 2004; pp 464-543.
(9) D'Hooge, D. R. In Silico Tracking of Individual Species Accelerating Progress in Macromolecular Engineering and Design. Macromol. Rapid Commun. 2018, 39 (14), 1800057. DOI: doi.org/10.1002/marc.201800057 (accessed 2023 Dec. 8).
(10) D'Hooge, D. R.; Van Steenberge, P. H. M.; Derboven, P.; Reyniers, M.-F.; Marin, G. B. Model-based design of the polymer microstructure: bridging the gap between Polym. Chem. and engineering. Polym. Chem. 2015, 6 (40), 7081-7096, 10.1039/C5PY01069A. DOI: 10.1039/C5PY01069A.
(11) Edeleva, M.; Marien, Y. W.; D'Hooge, D. R.; Van Steenberge, P. H. M. Exploiting (Multicomponent) Semibatch and Jacket Temperature Procedures to Safely Tune Molecular Properties for Solution Free Radical Polymerization of n-Butyl Acrylate. Macromol. Theory Simul. 2021, 30 (5), 2100024. DOI: doi.org/10.1002/mats.202100024 (accessed 2023 Dec. 8).
(12) Edeleva, M.; Marien, Y. W.; Van Steenberge, P. H. M.; D'Hooge, D. R. Jacket temperature regulation allowing well-defined non-adiabatic lab-scale solution free radical polymerization of acrylates. React. Chem. Eng. 2021, 6 (6), 1053-1069, 10.1039/D1RE00099C. DOI: 10.1039/D1RE00099C.
(13) Fierens, S. K.; Telitel, S.; Van Steenberge, P. H. M.; Reyniers, M.-F.; Marin, G. B.; Lutz, J.-F.; D'hooge, D. R. Model-Based Design To Push the Boundaries of Sequence Control. Macromolecules 2016, 49 (24), 9336-9344. DOI: 10.1021/acs.macromol.6b01699.
(14) Hernindez-Ortiz, J. C.; Van Steenberge, P. H. M.; Reyniers, M.-F.; Marin, G. B.; D'Hooge, D. R.; Duchateau, J. N. E.; Remerie, K.; Toloza, C.; Vaz, A. L.; Schreurs, F. Modeling the reaction event history and microstructure of individual macrospecies in postpolymerization modification. AIChE J. 2017, 63 (11), 4944-4961. DOI: doi.org/10.1002/aic.15842 (accessed 2023/12/08).
(15) Van Steenberge, P. H. M.; Sedlacek, O.; Hernindez-Ortiz, J. C.; Verbraeken, B.; Reyniers, M.-F.; Hoogenboom, R.; D'hooge, D. R. Visualization and design of the functional group distribution during statistical copolymerization. Nat. Commun. 2019, 10 (1), 3641. DOI: 10.1038/s41467-019-11368-6.
(16) De Smit, K.; Marien, Y. W.; Van Geem, K. M.; Van Steenberge, P. H. M.; D'Hooge, D. R. Connecting polymer synthesis and chemical recycling on a chain-by-chain basis: a unified matrix-based kinetic Monte Carlo strategy. React. Chem. Eng. 2020, 5 (10), 1909-1928, 10.1039/DORE00266F. DOI: 10.1039/DORE00266F.
(17) Gigmes, D.; Van Steenberge, P. H. M.; Siri, D.; D'Hooge, D. R.; Guillaneuf, Y.; Lefay, C. Simulation of the Degradation of Cyclic Ketene Acetal and Vinyl-Based Copolymers Synthesized via a Radical Process: Influence of the Reactivity Ratios on the Degradability Properties. Macromol. Rapid Commun. 2018, 39 (19), 1800193. DOI: doi.org/10.1002/marc.201800193 (accessed 2023 Dec. 8).
(18) Liausvia, F.; Rusli, W.; van Herk, A. Prediction of the Oligomer Distribution after Degradation of (Co)Polymers with Inserted Break Points. Macromol. Theory Simul. 2021, 30 (6), 2100038. DOI: doi.org/10.1002/mats.202100038 (accessed 2023 Dec. 8).
(19) Delplace, V.; Nicolas, J. Degradable vinyl polymers for biomedical applications. Nat. Chem. 2015, 7 (10), 771-784. DOI: 10.1038/nchem.2343.
(20) Martinez, M. A., Krzysztof%BJournal Name: CCS Chemistry; 4, J. V.; 7, J. I. Degradable and Recyclable Polymers by Reversible Deactivation Radical Polymerization. Journal Name: CCS Chemistry; Journal Volume: 4; Journal Issue: 7 2022, Medium: X.
(21) Zheng, J.; Png, Z. M.; Quek, X. C. N.; Loh, X. J.; Li, Z. Stimuli-cleavable moiety enabled vinyl polymer degradation and emerging applications. Green Chem. 2023, 25 (22), 8903-8934, 10.1039/D3GC03086E. DOI: 10.1039/D3GC03086E.
(22) Arroyave, A.; Cui, S.; Lopez, J. C.; Kocen, A. L.; LaPointe, A. M.; Delferro, M.; Coates, G. W. Catalytic Chemical Recycling of Post-Consumer Polyethylene. J. Am. Chem. Soc. 2022, 144 (51), 23280-23285. DOI: 10.1021/jacs.2c11949.
(23) Chen, L.; Malollari, K. G.; Uliana, A.; Hartwig, J. F. Ruthenium-Catalyzed, Chemoselective and Regioselective Oxidation of Polyisobutene. J. Am. Chem. Soc. 2021, 143 (12), 4531-4535. DOI: 10.1021/jacs.1c00125.
(24) Jia, X.; Qin, C.; Friedberger, T.; Guan, Z.; Huang, Z. Efficient and selective degradation of polyethylenes into liquid fuels and waxes under mild conditions. Sci. Adv. 2 (6), e1501591. DOI: 10.1126/sciadv.1501591 (accessed 2023 Dec. 8).
(25) Zeng, M.; Lee, Y.-H.; Strong, G.; LaPointe, A. M.; Kocen, A. L.; Qu, Z.; Coates, G. W.; Scott, S. L.; Abu-Omar, M. M. Chemical Upcycling of Polyethylene to Value-Added α,ω-Divinyl-Functionalized Oligomers. ACS Sustain. Chem. Eng. 2021, 9 (41), 13926-13936. DOI: 10.1021/acssuschemeng.1c05272.
(26) Fujioka, T.; Taketani, S.; Nagasaki, T.; Matsumoto, A. Self-Assembly and Cellular Uptake of Degradable and Water-Soluble Polyperoxides. Bioconjugate Chem. 2009, 20 (10), 1879-1887. DOI: 10.1021/bc9001618.
(27) Hatakenaka, H.; Takahashi, Y.; Matsumoto, A. Degradable Polymers Prepared from Alkyl Sorbates and Oxygen under Atmospheric Conditions and Precise Evaluation of Their Thermal Properties. Polym. J. 2003, 35 (8), 640-651. DOI: 10.1295/polymj.35.640.
(28) Matsumoto, A.; Higashi, H. Convenient Synthesis of Polymers Containing Labile Bonds in the Main Chain by Radical Alternating Copolymerization of Alkyl Sorbates with Oxygen. Macromolecules 2000, 33 (5), 1651-1655. DOI: 10.1021/ma990697c.
(29) Matsumoto, A.; Taketani, S. Regiospecific Radical Polymerization of a Tetrasubstituted Ethylene Monomer with Molecular Oxygen for the Synthesis of a New Degradable Polymer. J. Am. Chem. Soc. 2006, 128 (14), 4566-4567. DOI: 10.1021/ja0580385.
(30) Mete, S.; Choudhury, N.; De, P. Degradable alternating polyperoxides from poly(ethylene glycol)-substituted styrenic monomers with water solubility and thermoresponsiveness. J. Polym. Sci. Part A: Polym. Chem. 2018, 56 (18), 2030-2038. DOI: doi.org/10.1002/pola.29089 (accessed 2023 Dec. 8).
(31) Mete, S.; Mukherjee, P.; Maiti, B.; Pal, S.; Ghorai, P. K.; De, P. Degradable Crystalline Polyperoxides from Fatty Acid Containing Styrenic Monomers. Macromolecules 2018, 51 (21), 8912-8921. DOI: 10.1021/acs.macromol.8b01981.
(32) Mukundan, T.; Kishore, K. Synthesis, characterization and reactivity of polymeric peroxides. Prog. Polym. Sci. 1990, 15 (3), 475-505. DOI: doi.org/10.1016/0079-6700(90)90004-K.
(33) Pal, S.; Banoth, B.; Rahithya, G.; Dhawan, A.; De, P. Copolyperoxides of 2-(acetoacetoxy)ethyl methacrylate with methyl methacrylate and styrene; Synthesis, characterization, thermal analysis, and reactivity ratios. Polymer 2012, 53 (13), 2583-2590. DOI: doi.org/10.1016/j.polymer.2012.04.013.
(34) Sato, E.; Kitamura, T.; Matsumoto, A. In situ Collapse of Phase-Separated Structure by Covalent Bond Cleavage at a Branching Point upon Heating. Macromol. Rapid Commun. 2008, 29 (24), 1950-1953. DOI: doi.org/10.1002/marc.200800527 (accessed 2023 Dec. 8).
(35) Sato, E.; Matsumoto, A. Facile synthesis of functional polyperoxides by radical alternating copolymerization of 1,3-dienes with oxygen. Chem. Rec. 2009, 9 (5), 247-257. DOI: doi.org/10.1002/tcr.200900009 (accessed 2023 Dec. 8).
(36) Subramanian, K. Formation, Degradation, and Applications of Polyperoxides. Journal of Macromolecular Science, Part C 2003, 43 (3), 323-383. DOI: 10.1081/MC-120023910.
(37) Sugimoto, Y.; Taketani, S.; Kitamura, T.; Uda, D.; Matsumoto, A. Regiospecific Structure, Degradation, and Functionalization of Polyperoxides Prepared from Sorbic Acid Derivatives with Oxygen. Macromolecules 2006, 39 (26), 9112-9119. DOI: 10.1021/ma061823x.
(38) Taketani, S.; Matsumoto, A. Facile Synthesis of a Degradable Gel by Radical Copolymerization of Vinyl Sorbate and Molecular Oxygen. Macromol. Chem. Phys. 2004, 205 (18), 2451-2456. DOI: doi.org/10.1002/macp.200400386 (accessed 2023 Dec. 8).
(39) Adili, A.; Korpusik, A. B.; Seidel, D.; Sumerlin, B. S. Photocatalytic Direct Decarboxylation of Carboxylic Acids to Derivatize or Degrade Polymers. Angew. Chem. Int. Ed. 2022, 61 (40), e202209085. DOI: doi.org/10.1002/anie.202209085 (accessed 2023 Dec. 8).
(40) Garrison, J. B.; Hughes, R. W.; Sumerlin, B. S. Backbone Degradation of Polymethacrylates via Metal-Free Ambient-Temperature Photoinduced Single-Electron Transfer. ACS Macro Lett. 2022, 11 (4), 441-446. DOI: 10.1021/acsmacrolett.2c00091.
(41) Kimura, T.; Kuroda, K.; Kubota, H.; Ouchi, M. Metal-Catalyzed Switching Degradation of Vinyl Polymers via Introduction of an “In-Chain” Carbon-Halogen Bond as the Trigger. ACS Macro Lett. 2021, 10 (12), 1535-1539. DOI: 10.1021/acsmacrolett.1c00601.
(42) Makino, H.; Nishikawa, T.; Ouchi, M. Incorporation of a boryl pendant as the trigger in a methacrylate polymer for backbone degradation. Chem. Commun. 2022, 58 (85), 11957-11960, 10.1039/D2CC04882E. DOI: 10.1039/D2CC04882E.
(43) Makino, H.; Nishikawa, T.; Ouchi, M. Polymer Degradation by Synergistic Dual Stimuli: Base Interaction and Photocatalysis to Unlock a Boron Pendant Trigger for Main-Chain Scission. Macromolecules 2023, 56 (21), 8776-8783. DOI: 10.1021/acs.macromol.3c01165.
(44) Sano, Y.; Konishi, T.; Sawamoto, M.; Ouchi, M. Controlled radical depolymerization of chlorine-capped PMMA via reversible activation of the terminal group by ruthenium catalyst. European Polym. J. 2019, 120, 109181. DOI: doi.org/10.1016/j.eurpolymj.2019.08.008.
(45) Yamamoto, S.; Kubo, T.; Satoh, K. Interlocking degradation of vinyl polymers via main-chain C D C bonds scission by introducing pendant-responsive comonomers. J. Polym. Sci. 2022, 60 (24), 3435-3446. DOI: doi.org/10.1002/pol.20220250 (accessed 2023 Dec. 8).
(46) Pesenti, T.; Nicolas, J. 100th Anniversary of Macromolecular Science Viewpoint: Degradable Polymers from Radical Ring-Opening Polymerization: Latest Advances, New Directions, and Ongoing Challenges. ACS Macro Lett. 2020, 9 (12), 1812-1835. DOI: 10.1021/acsmacrolett.0c00676.
(47) Tardy, A.; Nicolas, J.; Gigmes, D.; Lefay, C.; Guillaneuf, Y. Radical Ring-Opening Polymerization: Scope, Limitations, and Application to (Bio)Degradable Materials. Chem. Rev. 2017, 117 (3), 1319-1406. DOI: 10.1021/acs.chemrev.6b00319.
(48) Bailey, W. J.; Ni, Z.; Wu, S.-R. Synthesis of poly-ε-caprolactone via a free radical mechanism. Free radical ring-opening polymerization of 2-methylene-1,3-dioxepane. J. Polym. Sci.: Polym. Chem. Edition 1982, 20 (11), 3021-3030. DOI: doi.org/10.1002/pol.1982.170201101 (accessed 2023 Dec. 8).
(49) Bailey, W. J.; Wu, S.-R.; Ni, Z. Synthesis and free radical ring-opening polymerization of 2-methylene-4-phenyl-1,3-dioxolane. Die Makromolekulare Chemie 1982, 183 (8), 1913-1920. DOI: doi.org/10.1002/macp.1982.021830811 (accessed 2023 Dec. 8).
(50) Bailey, W. J.; Wu, S.-R.; Ni, Z. Free Radical Ring-Opening Polymerization of 4-n-Hexyl- and 4-n-Decyl-2-methylene-1,3-dioxolanes. Journal of Macromolecular Science: Part A—Chemistry 1982, 18 (6), 973-986. DOI: 10.1080/00222338208077212.
(51) Do, P. T.; Poad, B. L. J.; Frisch, H. Programming Photodegradability into Vinylic Polymers via Radical Ring-Opening Polymerization. Angew. Chem. Int. Ed. 2023, 62 (6), e202213511. DOI: doi.org/10.1002/anie.202213511 (accessed 2023 Dec. 8).
(52) Evans, R. A.; Moad, G.; Rizzardo, E.; Thang, S. H. New Free-Radical Ring-Opening Acrylate Monomers. Macromolecules 1994, 27 (26), 7935-7937. DOI: 10.1021/ma00104a062.
(53) Evans, R. A.; Rizzardo, E. Free-Radical Ring-Opening Polymerization of Cyclic Allylic Sulfides. Macromolecules 1996, 29 (22), 6983-6989. DOI: 10.1021/ma960573p.
(54) Evans, R. A.; Rizzardo, E. Free-Radical Ring-Opening Polymerization of Cyclic Allylic Sulfides. 2. Effect of Substituents on Seven- and Eight-Membered Ring Low Shrink Monomers. Macromolecules 2000, 33 (18), 6722-6731. DOI: 10.1021/ma9917646.
(55) Huang, H.; Sun, B.; Huang, Y.; Niu, J. Radical Cascade-Triggered Controlled Ring-Opening Polymerization of Macrocyclic Monomers. J. Am. Chem. Soc. 2018, 140 (33), 10402-10406. DOI: 10.1021/jacs.8b05365.
(56) Paulusse, J. M. J.; Amir, R. J.; Evans, R. A.; Hawker, C. J. Free Radical Polymers with Tunable and Selective Bio- and Chemical Degradability. J. Am. Chem. Soc. 2009, 131 (28), 9805-9812. DOI: 10.1021/ja903245p.
(57) Phelan, M.; Aldabbagh, F.; Zetterlund, P. B.; Yamada, B. Mechanism and kinetics of the free radical ring-opening polymerization of cyclic allylic sulfide lactones. Polymer 2005, 46 (26), 12046-12056. DOI: doi.org/10.1016/j.polymer.2005.11.006.
(58) Sbordone, F.; Veskova, J.; Richardson, B.; Do, P. T.; Micallef, A.; Frisch, H. Embedding Peptides into Synthetic Polymers: Radical Ring-Opening Copolymerization of Cyclic Peptides. J. Am. Chem. Soc. 2023, 145 (11), 6221-6229. DOI: 10.1021/jacs.2c12517.
(59) Wang, W.; Rondon, B.; Wang, Z.; Wang, J.; Niu, J. Macrocyclic Allylic Sulfone as a Universal Comonomer in Organocatalyzed Photocontrolled Radical Copolymerization with Vinyl Monomers. Macromolecules 2023, 56 (5), 2052-2061. DOI: 10.1021/acs.macromol.2c02025.
(60) Wang, W.; Zhou, Z.; Sathe, D.; Tang, X.; Moran, S.; Jin, J.; Haeffner, F.; Wang, J.; Niu, J. Degradable Vinyl Random Copolymers via Photocontrolled Radical Ring-Opening Cascade Copolymerization**. Angew. Chem. Int. Ed. 2022, 61 (8), e202113302. DOI: doi.org/10.1002/anie.202113302 (accessed 2023 Dec. 8).
(61) Agarwal, S. Microstructural Characterisation and Properties Evaluation of Poly (methyl methacrylate-co-ester)s. Polym. J. 2007, 39 (2), 163-174. DOI: 10.1295/polymj.PJ2006137.
(62) Endo, T.; Yako, N.; Azuma, K.; Nate, K. Ring-opening polymerization of 2-methylene-4-phenyl-1,3-dioxolane. Die Makromolekulare Chemie 1985, 186 (8), 1543-1548. DOI: doi.org/10.1002/macp.1985.021860802 (accessed 2023 Dec. 8).
(63) Grabe, N.; Zhang, Y.; Agarwal, S. Degradable Elastomeric Block Copolymers Based on Polycaprolactone by Free-Radical Chemistry. Macromol. Chem. Phys. 2011, 212 (13), 1327-1334. DOI: doi.org/10.1002/macp.201100031 (accessed 2023 Dec. 8).
(64) Guégain, E.; Michel, J.-P.; Boissenot, T.; Nicolas, J. Tunable Degradation of Copolymers Prepared by Nitroxide-Mediated Radical Ring-Opening Polymerization and Point-by-Point Comparison with Traditional Polyesters. Macromolecules 2018, 51 (3), 724-736. DOI: 10.1021/acs.macromol.7b02655.
(65) Hiracuri, Y.; Tokiwa, Y. Synthesis of copolymers composed of 2-methylene-1,3,6-trioxocane and vinyl monomers and their enzymatic degradation. J. Polym. Sci. Part A: Polym. Chem. 1993, 31 (12), 3159-3163. DOI: doi.org/10.1002/pola.1993.080311233 (accessed 2023 Dec. 8).
(66) Jin, Q.; Maji, S.; Agarwal, S. Novel amphiphilic, biodegradable, biocompatible, cross-linkable copolymers: synthesis, characterization and drug delivery applications. Polym. Chem. 2012, 3 (10), 2785-2793, 10.1039/C2PY20364B. DOI: 10.1039/C2PY20364B.
(67) Ko, J. H.; Terashima, T.; Sawamoto, M.; Maynard, H. D. Fluorous Comonomer Modulates the Reactivity of Cyclic Ketene Acetal and Degradation of Vinyl Polymers. Macromolecules 2017, 50 (23), 9222-9232. DOI: 10.1021/acs.macromol.7b01973.
(68) Lai, H.; Ouchi, M. Backbone-Degradable Polymers via Radical Copolymerizations of Pentafluorophenyl Methacrylate with Cyclic Ketene Acetal: Pendant Modification and Efficient Degradation by Alternating-Rich Sequence. ACS Macro Lett. 2021, 10 (10), 1223-1228. DOI: 10.1021/acsmacrolett.1c00513.
(69) Lau, U. Y.; Pelegri-O'Day, E. M.; Maynard, H. D. Synthesis and Biological Evaluation of a Degradable Trehalose Glycopolymer Prepared by RAFT Polymerization. Macromol. Rapid Commun. 2018, 39 (5), 1700652. DOI: doi.org/10.1002/marc.201700652 (accessed 2023 Dec. 8).
(70) Lutz, J.-F.; Andrieu, J.; Üzgiin, S.; Rudolph, C.; Agarwal, S. Biocompatible, Thermoresponsive, and Biodegradable: Simple Preparation of “All-in-One” Biorelevant Polymers. Macromolecules 2007, 40 (24), 8540-8543. DOI: 10.1021/ma7021474.
(71) Pesenti, T.; Zhu, C.; Gonzalez-Martinez, N.; Tomás, R. M. F.; Gibson, M. I.; Nicolas, J. Degradable Polyampholytes from Radical Ring-Opening Copolymerization Enhance Cellular Cryopreservation. ACS Macro Lett. 2022, 11 (7), 889-894. DOI: 10.1021/acsmacrolett.2c00298.
(72) Roberts, G. E.; Coote, M. L.; Heuts, J. P. A.; Morris, L. M.; Davis, T. P. Radical Ring-Opening Copolymerization of 2-Methylene 1,3-Dioxepane and Methyl Methacrylate: Experiments Originally Designed To Probe the Origin of the Penultimate Unit Effect. Macromolecules 1999, 32 (5), 1332-1340. DOI: 10.1021/ma9813587.
(73) Seema, A.; Liqun, R. Polycaprolactone-Based Novel Degradable Ionomers by Radical Ring-Opening Polymerization of 2-Methylene-1,3-dioxepane. Macromolecules 2009, 42 (5), 1574-1579. DOI: 10.1021/ma802615f.
(74) Wickel, H.; Agarwal, S.; Greiner, A. Homopolymers and Random Copolymers of 5,6-Benzo-2-methylene-1,3-dioxepane and Methyl Methacrylate: Structural Characterization Using 1D and 2D NMR. Macromolecules 2003, 36 (7), 2397-2403. DOI: 10.1021/ma025983u.
(75) Zhang, Y.; Aigner, A.; Agarwal, S. Degradable and Biocompatible Poly(N,N-dimethylaminoethyl Methacrylate-co-caprolactone)s as DNA Transfection Agents. Macromolecular Bioscience 2013, 13 (9), 1267-1275. DOI: doi.org/10.1002/mabi.201300043 (accessed 2023 Dec. 8). Zhang, Y.; Chu, D.; Zheng, M.; Kissel, T.; Agarwal, S. Biocompatible and degradable poly(2-hydroxyethyl methacrylate) based polymers for biomedical applications. Polym. Chem. 2012, 3 (10), 2752-2759, 10.1039/C2PY20403G. DOI: 10.1039/C2PY20403G.
(76) Zhang, Y.; Zheng, M.; Kissel, T.; Agarwal, S. Design and Biophysical Characterization of Bioresponsive Degradable Poly(dimethylaminoethyl methacrylate) Based Polymers for In Vitro DNA Transfection. Biomacromolecules 2012, 13 (2), 313-322. DOI: 10.1021/bm2015174.
(77) Bingham, N. M.; Abousalman-Rezvani, Z.; Collins, K.; Roth, P. J. Thiocarbonyl chemistry in polymer science. Polym. Chem. 2022, 13 (20), 2880-2901. DOI: doi.org/10.1039/d2py00050d.
(78) Bingham, N. M.; Roth, P. J. Degradable vinyl copolymers through thiocarbonyl addition-ring-opening (TARO) polymerization. Chem. Commun. 2019, 55 (1), 55-58, 10.1039/C8CC08287A. DOI: 10.1039/C8CC08287A.
(79) Smith, R. A.; Fu, G.; McAteer, O.; Xu, M.; Gutekunst, W. R. Radical Approach to Thioester-Containing Polymers. J. Am. Chem. Soc. 2019, 141 (4), 1446-1451. DOI: 10.1021/jacs.8b12154.
(80) Bingham, N. M.; Nisa, Q. u.; Chua, S. H. L.; Fontugne, L.; Spick, M. P.; Roth, P. J. Thioester-Functional Polyacrylamides: Rapid Selective Backbone Degradation Triggers Solubility Switch Based on Aqueous Lower Critical Solution Temperature/Upper Critical Solution Temperature. ACS Appl. Polym. Mater. 2020, 2 (8), 3440-3449. DOI: 10.1021/acsapm.0c00503.
(81) Bingham, N. M.; Nisa, Q. u.; Gupta, P.; Young, N. P.; Velliou, E.; Roth, P. J. Biocompatibility and Physiological Thiolytic Degradability of Radically Made Thioester-Functional Copolymers: Opportunities for Drug Release. Biomacromolecules 2022, 23 (5), 2031-2039. DOI: 10.1021/acs.biomac.2c00039.
(82) Galanopoulo, P.; Gil, N.; Gigmes, D.; Lefay, C.; Guillaneuf, Y.; Lages, M.; Nicolas, J.; D'Agosto, F.; Lansalot, M. RAFT-Mediated Emulsion Polymerization-Induced Self-Assembly for the Synthesis of Core-Degradable Waterborne Particles. Angew. Chem. Int. Ed. 2023, 62 (16), e202302093. DOI: doi.org/10.1002/anie.202302093 (accessed 2023 Dec. 8).
(83) Galanopoulo, P.; Gil, N.; Gigmes, D.; Lefay, C.; Guillaneuf, Y.; Lages, M.; Nicolas, J.; Lansalot, M.; D'Agosto, F. One-Step Synthesis of Degradable Vinylic Polymer-Based Latexes via Aqueous Radical Emulsion Polymerization. Angew. Chem. Int. Ed. 2022, 61 (15), e202117498. DOI: doi.org/10.1002/anie.202117498 (accessed 2023 Dec. 8).
(84) Lages, M.; Gil, N.; Galanopoulo, P.; Mougin, J.; Lefay, C.; Guillaneuf, Y.; Lansalot, M.; D'Agosto, F.; Nicolas, J. Degradable Vinyl Copolymer Nanoparticles/Latexes by Aqueous Nitroxide-Mediated Polymerization-Induced Self-Assembly. Macromolecules 2022, 55 (21), 9790-9801. DOI: 10.1021/acs.macromol.2c01734.
(85) Luzel, B.; Gil, N.; Désirée, P.; Monot, J.; Bourissou, D.; Siri, D.; Gigmes, D.; Martin-Vaca, B.; Lefay, C.; Guillaneuf, Y. Development of an Efficient Thionolactone for Radical Ring-Opening Polymerization by a Combined Theoretical/Experimental Approach. J. Am. Chem. Soc. 2023. DOI: 10.1021/jacs.3c08610.
(86) un Nisa, Q.; Theobald, W.; Hepburn, K. S.; Riddlestone, I.; Bingham, N. M.; Kopeć, M.; Roth, P. J. Degradable Linear and Bottlebrush Thioester-Functional Copolymers through Atom-Transfer Radical Ring-Opening Copolymerization of a Thionolactone. Macromolecules 2022, 55 (17), 7392-7400. DOI: 10.1021/acs.macromol.2c01317.
(87) Kiel, G. R.; Lundberg, D. J.; Prince, E.; Husted, K. E. L.; Johnson, A. M.; Lensch, V.; Li, S.; Shieh, P.; Johnson, J. A. Cleavable Comonomers for Chemically Recyclable Polystyrene: A General Approach to Vinyl Polymer Circularity. J. Am. Chem. Soc. 2022, 144 (28), 12979-12988. DOI: 10.1021/jacs.2c05374.
(88) Rix, M. F. I.; Collins, K.; Higgs, S. J.; Dodd, E. M.; Coles, S. J.; Bingham, N. M.; Roth, P. J. Insertion of Degradable Thioester Linkages into Styrene and Methacrylate Polymers: Insights into the Reactivity of Thionolactones. Macromolecules 2023. DOI: 10.1021/acs.macromol.3c01811.
(89) Goseki, R.; Ishizone, T. Poly(methyl methacrylate) (PMMA). In Encyclopedia of Polymeric Nanomaterials, Kobayashi, S., Mullen, K. Eds.; Springer Berlin Heidelberg, 2021; pp 1-11.
(90) De Tommaso, J.; Dubois, J.-L. Risk Analysis on PMMA Recycling Economics. In Polymers, 2021; Vol. 13.
(91) Meyer, V. E.; Lowry, G. G. Integral and differential binary copolymerization equations. J. Polym. Sci. Part A Gen. Pap. 1965, 3 (8), 2843-2851. DOI: doi.org/10.1002/pol.1965.100030811 (accessed 2023 Dec. 8).
(92) Lynd, N. A.; Ferrier, R. C., Jr.; Beckingham, B. S. Recommendation for Accurate Experimental Determination of Reactivity Ratios in Chain Copolymerization. Macromolecules 2019, 52 (6), 2277-2285. DOI: 10.1021/acs.macromol.8b01752.
(93) WebMat: Material Property Data. matweb.com/search/datasheet.aspx?bassnum=O1303&ckck=1 (accessed 2023 Dec. 19).
(94) Bellotti, V.; Parkatzidis, K.; Wang, H. S.; De Alwis Watuthanthrige, N.; Orfano, M.; Monguzzi, A.; Truong, N. P.; Simonutti, R.; Anastasaki, A. Light-accelerated depolymerization catalyzed by Eosin Y. Polymer Chemistry 2023, 14 (3), 253-258, 10.1039/D2PY01383E. DOI: 10.1039/D2PY01383E.
(95) Bellotti, V.; Wang, H. S.; Truong, N. P.; Simonutti, R.; Anastasaki, A. Temporal Regulation of PET-RAFT Controlled Radical Depolymerization. Angewandte Chemie International Edition 2023, 62 (45), e202313232. DOI: doi.org/10.1002/anie.202313232 (accessed 2024 Feb. 10).
(96) De Luca Bossa, F.; Yilmaz, G.; Matyjaszewski, K. Fast Bulk Depolymerization of Polymethacrylates by ATRP. ACS Macro Letters 2023, 12 (8), 1173-1178. DOI: 10.1021/acsmacrolett.3c00389.
(97) Flanders, M. J.; Gramlich, W. M. Reversible-addition fragmentation chain transfer (RAFT) mediated depolymerization of brush polymers. Polymer Chemistry 2018, 9 (17), 2328-2335, 10.1039/C8PY00446C. DOI: 10.1039/C8PY00446C.
(98) Gkaliou, K.; Benedini, L.; Sárossy, Z.; Dalsgaard Jensen, C.; Henriksen, U. B.; Daugaard, A. E. Recycled PMMA prepared directly from crude MMA obtained from thermal depolymerization of mixed PMMA waste. Waste Management 2023, 164, 191-199. DOI: doi.org/10.1016/j.wasman.2023.04.007.
(99) Martinez, M. R.; Dadashi-Silab, S.; Lorandi, F.; Zhao, Y.; Matyjaszewski, K. Depolymerization of P(PDMS11MA) Bottlebrushes via Atom Transfer Radical Polymerization with Activator Regeneration. Macromolecules 2021, 54 (12), 5526-5538. DOI: 10.1021/acs.macromol.1c00415.
(100) Martinez, M. R.; De Luca Bossa, F.; Olszewski, M.; Matyjaszewski, K. Copper(II) Chloride/Tris(2-pyridylmethyl)amine-Catalyzed Depolymerization of Poly(n-butyl methacrylate). Macromolecules 2022, 55 (1), 78-87. DOI: 10.1021/acs.macromol.1c02246.
(101) Martinez, M. R.; Schild, D.; De Luca Bossa, F.; Matyjaszewski, K. Depolymerization of Polymethacrylates by Iron ATRP. Macromolecules 2022, 55 (23), 10590-10599. DOI: 10.1021/acs.macromol.2c01712.
(102) Wang, H. S.; Parkatzidis, K.; Junkers, T.; Truong, N. P.; Anastasaki, A. Controlled radical depolymerization: Structural differentiation and molecular weight control. Chem 2024, 10 (1), 388-401. DOI: doi.org/10.1016/j.chempr.2023.09.027.
(103) Wang, H. S.; Truong, N. P.; Jones, G. R.; Anastasaki, A. Investigating the Effect of End-Group, Molecular Weight, and Solvents on the Catalyst-Free Depolymerization of RAFT Polymers: Possibility to Reverse the Polymerization of Heat-Sensitive Polymers. ACS Macro Letters 2022, 11 (10), 1212-1216. DOI: 10.1021/acsmacrolett.2c00506.
(104) Wang, H. S.; Truong, N. P.; Pei, Z.; Coote, M. L.; Anastasaki, A. Reversing RAFT Polymerization: Near-Quantitative Monomer Generation Via a Catalyst-Free Depolymerization Approach. Journal of the American Chemical Society 2022, 144 (10), 4678-4684. DOI: 10.1021/jacs.2c00963.
(105) Whitfield, R.; Jones, G. R.; Truong, N. P.; Manring, L. E.; Anastasaki, A. Solvent-Free Chemical Recycling of Polymethacrylates made by ATRP and RAFT polymerization: High-Yielding Depolymerization at Low Temperatures. Angewandte Chemie International Edition 2023, 62 (38), e202309116. DOI: doi.org/10.1002/anie.202309116 (accessed 2024 Feb. 10).
(106) Young, J. B.; Bowman, J. I.; Eades, C. B.; Wong, A. J.; Sumerlin, B. S. Photoassisted Radical Depolymerization. ACS Macro Letters 2022, 11 (12), 1390-1395. DOI: 10.1021/acsmacrolett.2c00603.
(107) Young, J. B.; Hughes, R. W.; Tamura, A. M.; Bailey, L. S.; Stewart, K. A.; Sumerlin, B. S. Bulk depolymerization of poly(methyl methacrylate) via chain-end initiation for catalyst-free reversion to monomer. Chem 2023, 9 (9), 2669-2682. DOI: doi.org/10.1016/j.chempr.2023.07.004.
(108) Neese, F. Software update: The ORCA program system-Version 5.0. WIREs Comput. Mol. Sci. 2022, 12, e1606.
(109) Chai, J.-D.; Head-Gordon, M. Systematic optimization of long-range corrected hybrid density functionals. J. Chem. Phys. 2008, 128, 084106.
(110) Grimme, S.; Antony, J.; Ehrlich, S.; Krieg, H. A consistent and accurate ab initio parametrization of density functional dispersion correction (DFT-D) for the 94 elements H—Pu. J. Chem. Phys. 2010, 132, 154104.
(111) Schäfer, A.; Horn, H.; Ahlrichs, R. Fully optimized contracted Gaussian basis sets for atoms Li to Kr. J. Chem. Phys. 1992, 97, 2571-2577.
(112) Weigend, F.; Ahlrichs, R. Balanced basis sets of split valence, triple zeta valence and quadruple zeta valence quality for H to Rn: Design and assessment of accuracy. Phys. Chem. Chem. Phys. 2005, 7, 3297-3305.
(113) Neese, F. An improvement of the resolution of the identity approximation for the formation of the Coulomb matrix. J. Comput. Chem. 2003, 24, 1740-1747.
(114) Neese, F.; Wennmohs, F.; Hansen, A.; Becker, U. Efficient, approximate and parallel Hartree-Fock and hybrid DFT calculations. A ‘chain-of-spheres’ algorithm for the Hartree-Fock exchange. Chem. Phys. 2009, 356, 98-109.
(115) Barone, V.; Cossi, M. Quantum Calculation of Molecular Energies and Energy Gradients in Solution by a Conductor Solvent Model. J. Phys. Chem. A 1998, 102, 1995-2001.
(116) Cossi, M.; Rega, N.; Scalmani, G.; Barone, V. Energies, structures, and electronic properties of molecules in solution with the C-PCM solvation model. J. Comput. Chem. 2003, 24, 669-681.
(117) Dai, X.-J.; Engl, O. D.; León, T.; Buchwald, S. L. Catalytic Asymmetric Synthesis of α-Arylpyrrolidines and Benzo-fused Nitrogen Heterocycles. Angew. Chem. Int. Ed. 2019, 58, 3407-3411.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of the Formula II: 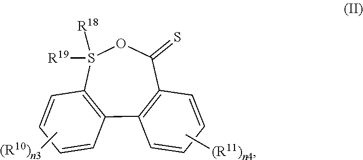
or a tautomer or salt thereof, wherein:
each instance of R¹⁰ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R¹¹ is independently halogen, substituted or unsubstituted alkyl, substituted or unsubstituted carbocyclyl, substituted or unsubstituted heterocyclyl, substituted or unsubstituted aryl, substituted or unsubstituted heteroaryl, —CN, —OR^{b}, —SCN, —SR^{b}, —SSR^{b}, —N₃, —NO, —N(R^{b})₂, —NO₂, —C(═O)R^{b}, —C(═O)OR^{b}, —C(═O)SR^{b}, —C(═O)N(R^{b})₂, —C(═NR^{b})R^{b}, —C(═NR^{b})OR^{b}, —C(═NR^{b})SR^{b}, —C(═NR^{b})N(R^{b})₂, —S(═O)R^{b}, —S(═O)OR^{b}, —S(═O)SR^{b}, —S(═O)N(R^{b})₂, —S(═O)₂R^{b}, —S(═O)₂OR^{b}, —S(═O)₂SR^{b}, —S(═O)₂N(R^{b})₂, —OC(═O)R^{b}, —OC(═O)OR^{b}, —OC(═O)SR^{b}, —OC(═O)N(R^{b})₂, —OC(═NR^{b})R^{b}, —OC(═NR^{b})OR^{b}, —OC(═NR^{b})SR^{b}, —OC(═NR^{b})N(R^{b})₂, —OS(═O)R^{b}, —OS(═O)OR^{b}, —OS(═O)SR^{b}, —OS(═O)N(R^{b})₂, —OS(═O)₂R^{b}, —OS(═O)₂OR^{b}, —OS(═O)₂SR^{b}, —OS(═O)₂N(R^{b})₂, —ON(R^{b})₂, —SC(═O)R^{b}, —SC(═O)OR^{b}, —SC(═O)SR^{b}, —SC(═O)N(R^{b})₂, —SC(═NR^{b})R^{b}, —SC(═NR^{b})OR^{b}, —SC(═NR^{b})SR^{b}, —SC(═NR^{b})N(R^{b})₂, —NR^{b}C(═O)R^{b}, —NR^{b}C(═O)OR^{b}, —NR^{b}C(═O)SR^{b}, —NR^{b}C(═O)N(R^{b})₂, —NR^{b}C(═NR^{b})R^{b}, —NR^{b}C(═NR^{b})OR^{b}, —NR^{b}C(═NR^{b})SR^{b}, —NR^{b}C(═NR^{b})N(R^{b})₂, —NR^{b}S(═O)R^{b}, —NR^{b}S(═O)OR^{b}, —NR^{b}S(═O)SR^{b}, —NR^{b}S(═O)N(R^{b})₂, —NR^{b}S(═O)₂R^{b}, —NR^{b}S(═O)₂OR^{b}, —NR^{b}S(═O)₂SR^{b}, —NR^{b}S(═O)₂N(R^{b})₂, —Si(R^{b})₃, —Si(R^{b})₂OR^{b}, —Si(R^{b})(OR^{b})₂, —Si(OR^{b})₃, —OSi(R^{b})₃, —OSi(R^{b})₂OR^{b}, —OSi(R^{b})(OR^{b})₂, or —OSi(OR^{b})₃;
each instance of R^{b} is independently hydrogen, substituted or unsubstituted alkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;
each of n3 and n4 is independently 0, 1, 2, 3, or 4;
R¹⁸ is substituted or unsubstituted aryl, substituted or unsubstituted alkyl, substituted or unsubstituted carbocyclyl, substituted or unsubstituted heterocyclyl, or substituted or unsubstituted heteroaryl; and
R¹⁹ is hydrogen, substituted or unsubstituted alkyl, substituted or unsubstituted carbocyclyl, substituted or unsubstituted heterocyclyl, substituted or unsubstituted aryl, or substituted or unsubstituted heteroaryl;
provided that no instance of R¹⁰, R¹¹, R¹⁸, and R¹⁹ comprises one or more non-aromatic unsaturated CC bonds.

2. The compound of claim 1, or a tautomer or salt thereof, wherein the compound is of the formula:

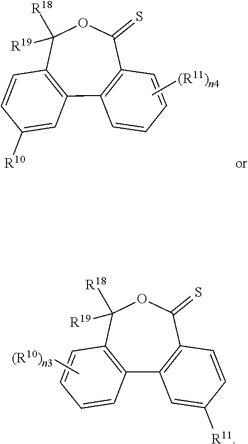

3. The compound of claim 1, or a tautomer or salt thereof, wherein the compound is of the formula:

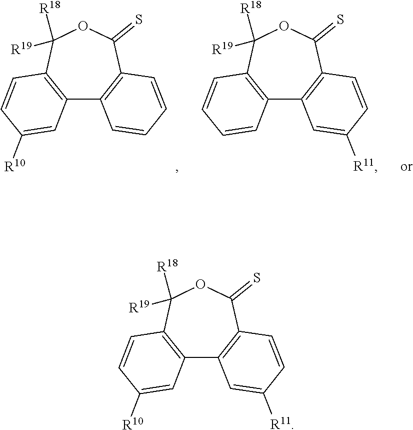

4. The compound of claim 1, or a tautomer or salt thereof, wherein n3 is 1.

5. The compound of claim 1, or a tautomer or salt thereof, wherein n3 is 0.

6-19. (canceled)

20. The compound of claim 1, wherein the compound is of the formula: 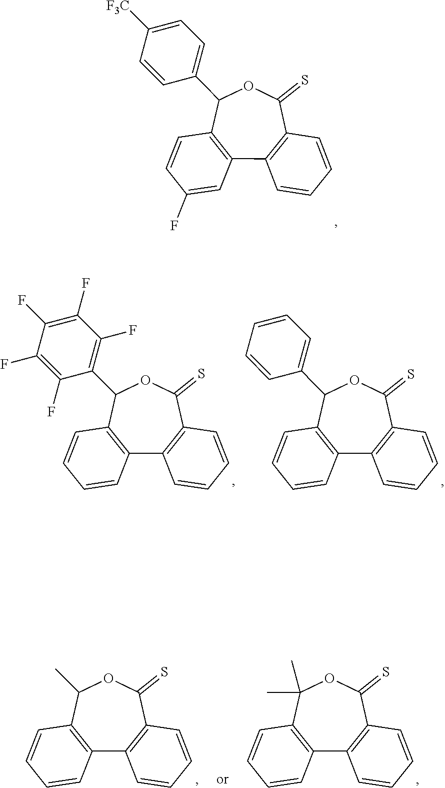
or a tautomer or salt thereof.

21. The compound of claim 1, wherein the compound is of the formula: 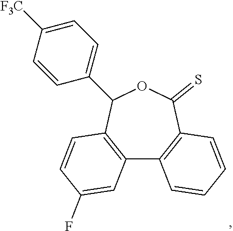 [SMILES: Fc1ccc2c(c1)-c1ccccc1C(=S)OC2c1ccc(C(F)(F)F)cc1]
or a tautomer or salt thereof.

22. The compound of claim 1, wherein the compound is of the formula: 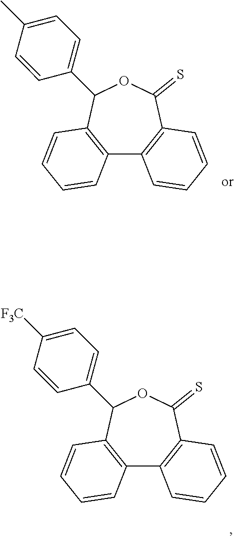 [SMILES: Cc1ccc(C2OC(=S)c3ccccc3-c3ccccc32)cc1 ; FC(F)(F)c1ccc(C2OC(=S)c3ccccc3-c3ccccc32)cc1]
or a tautomer or salt thereof.

23-93. (canceled)

94. The compound of claim 2, or a tautomer or salt thereof, wherein n3 is 1.

95. The compound of claim 2, or a tautomer or salt thereof, wherein n3 is 0.
